SEQUENCE LISTING (161 sequences)

SEQ ID NO: 1 - DNA, 44 bp, synthetic construct
  source 1..44: mol_type = other DNA
tttctctatcgataggtcatgtttgtagacttcccaaatttgcc

SEQ ID NO: 2 - DNA, 38 bp, synthetic construct
  source 1..38: mol_type = other DNA
ccggaatgccaagctaacttggagaagggaagtcacgg

SEQ ID NO: 3 - DNA, 36 bp, synthetic construct
  source 1..36: mol_type = other DNA
tttctctatcgataggcgcagctaaaactggtgaat

SEQ ID NO: 4 - DNA, 36 bp, synthetic construct
  source 1..36: mol_type = other DNA
ccggaatgccaagctgcgtctgaggagactctgagt

SEQ ID NO: 5 - DNA, 43 bp, synthetic construct
  source 1..43: mol_type = other DNA
tttctctatcgatagaaacccataaaattcatttcctaggggt

SEQ ID NO: 6 - DNA, 34 bp, synthetic construct
  source 1..34: mol_type = other DNA
ccggaatgccaagctgagagcagcaggaggaggg

SEQ ID NO: 7 - DNA, 20 bp, synthetic construct
  source 1..20: mol_type = other DNA
agcttggcattccggtactg

SEQ ID NO: 8 - DNA, 27 bp, synthetic construct
  source 1..27: mol_type = other DNA
ctatcgatagagaaatgttctggcacc

SEQ ID NO: 9 - DNA, 21 bp, synthetic construct
  source 1..21: mol_type = other DNA
gtgtcactgttggaggttatt

SEQ ID NO: 10 - DNA, 21 bp, synthetic construct
  source 1..21: mol_type = other DNA
tggataagaccatgagtattg

SEQ ID NO: 11 - DNA, 21 bp, synthetic construct
  source 1..21: mol_type = other DNA
cccagagatgttagctgaaat

SEQ ID NO: 12 - DNA, 21 bp, synthetic construct
  source 1..21: mol_type = other DNA
tggataagtccatgagtattg

SEQ ID NO: 13 - DNA, 21 bp, synthetic construct
  source 1..21: mol_type = other DNA
ccagtttctaatatgctatac

SEQ ID NO: 14 - DNA, 22 bp, synthetic construct
  source 1..22: mol_type = other DNA
tcaaggagcagtttctggacgg

SEQ ID NO: 15 - DNA, 22 bp, synthetic construct
  source 1..22: mol_type = other DNA
gcatcctggcttgtctgcaaac

SEQ ID NO: 16 - DNA, 23 bp, synthetic construct
  source 1..23: mol_type = other DNA
gctggttagatgatgagcctgag

SEQ ID NO: 17 - DNA, 22 bp, synthetic construct
  source 1..22: mol_type = other DNA
acaccacatccaggagctgact

SEQ ID NO: 18 - DNA, 22 bp, synthetic construct
  source 1..22: mol_type = other DNA
gtctgtcagtgtgacccatcct

SEQ ID NO: 19 - DNA, 23 bp, synthetic construct
  source 1..23: mol_type = other DNA
ctgagcaggattttccacaggtg

SEQ ID NO: 20 - DNA, 22 bp, synthetic construct
  source 1..22: mol_type = other DNA
gtcagccacttgaagaagcagg

SEQ ID NO: 21 - DNA, 22 bp, synthetic construct
  source 1..22: mol_type = other DNA
taggaactcggagtgagcctca

SEQ ID NO: 22 - DNA, 22 bp, synthetic construct
  source 1..22: mol_type = other DNA
gagcctgttctcatcaccatgg

SEQ ID NO: 23 - DNA, 22 bp, synthetic construct
  source 1..22: mol_type = other DNA
gtaggcaaagctcaagtccagc

SEQ ID NO: 24 - DNA, 22 bp, synthetic construct
  source 1..22: mol_type = other DNA
gcagtcaactcctggaccacta

SEQ ID NO: 25 - DNA, 22 bp, synthetic construct
  source 1..22: mol_type = other DNA
caaggttcccactgcttacagc

SEQ ID NO: 26 - DNA, 22 bp, synthetic construct
  source 1..22: mol_type = other DNA
atgcccttcacaatagcagcgg

SEQ ID NO: 27 - DNA, 22 bp, synthetic construct
  source 1..22: mol_type = other DNA
ccaaaactgcgaacggtctgca

SEQ ID NO: 28 - DNA, 20 bp, synthetic construct
  source 1..20: mol_type = other DNA
ccgcacaacctcggcaggca

SEQ ID NO: 29 - DNA, 23 bp, synthetic construct
  source 1..23: mol_type = other DNA
cagaagcgatgaacagaagactc

SEQ ID NO: 30 - DNA, 21 bp, synthetic construct
  source 1..21: mol_type = other DNA
gcagtttgttgagccaagtcg

SEQ ID NO: 31 - DNA, 19 bp, synthetic construct
  source 1..19: mol_type = other DNA
ccagccgaatggagtcctt

SEQ ID NO: 32 - DNA, 22 bp, synthetic construct
  source 1..22: mol_type = other DNA
agatacacctgccatgtgcagc

SEQ ID NO: 33 - DNA, 22 bp, synthetic construct
  source 1..22: mol_type = other DNA
gatcacagctccaaggagaacc

SEQ ID NO: 34 - DNA, 23 bp, synthetic construct
  source 1..23: mol_type = other DNA
ctgctgtgatgtgtaggaggaag

SEQ ID NO: 35 - DNA, 22 bp, synthetic construct
  source 1..22: mol_type = other DNA
gctgtgagagacacatcagagc

SEQ ID NO: 36 - DNA, 22 bp, synthetic construct
  source 1..22: mol_type = other DNA
ggagacacagaagtacaagcgc

SEQ ID NO: 37 - DNA, 22 bp, synthetic construct
  source 1..22: mol_type = other DNA
acatcctctggagggtgtgaga

SEQ ID NO: 38 - DNA, 24 bp, synthetic construct
  source 1..24: mol_type = other DNA
ccactgaaaaagatgagtatgcct

SEQ ID NO: 39 - DNA, 23 bp, synthetic construct
  source 1..23: mol_type = other DNA
ccaatccaaatgcggcatcttca

SEQ ID NO: 40 - DNA, 21 bp, synthetic construct
  source 1..21: mol_type = other DNA
aaggtgaaggtcggagtcaac

SEQ ID NO: 41 - DNA, 21 bp, synthetic construct
  source 1..21: mol_type = other DNA
gaaggggtcattgatggcaac

SEQ ID NO: 42 - DNA, 24 bp, synthetic construct
  source 1..24: mol_type = other DNA
ccatcagcttgtctgtttcattcc

SEQ ID NO: 43 - DNA, 23 bp, synthetic construct
  source 1..23: mol_type = other DNA
gctgtaatccaaggaatgtggtc

SEQ ID NO: 44 - DNA, 22 bp, synthetic construct
  source 1..22: mol_type = other DNA
gtttcactgcctggtgttgagc

SEQ ID NO: 45 - DNA, 22 bp, synthetic construct
  source 1..22: mol_type = other DNA
acgacgggcacgctgaagtttg

SEQ ID NO: 46 - DNA, 22 bp, synthetic construct
  source 1..22: mol_type = other DNA
ctggctttcgtgtgctggagaa

SEQ ID NO: 47 - DNA, 22 bp, synthetic construct
  source 1..22: mol_type = other DNA
gctgtcagagtgtttcagaggc

SEQ ID NO: 48 - DNA, 22 bp, synthetic construct
  source 1..22: mol_type = other DNA
ctcacagatgctggcacctaca

SEQ ID NO: 49 - DNA, 22 bp, synthetic construct
  source 1..22: mol_type = other DNA
gcaaggtctctgagctggcatt

SEQ ID NO: 50 - DNA, 22 bp, synthetic construct
  source 1..22: mol_type = other DNA
cctggaggatgaagggtgttac

SEQ ID NO: 51 - DNA, 23 bp, synthetic construct
  source 1..23: mol_type = other DNA
agtgaagggatactatgggctgt

SEQ ID NO: 52 - DNA, 21 bp, synthetic construct
  source 1..21: mol_type = other DNA
ttcgcacaggctcagcagcag

SEQ ID NO: 53 - DNA, 22 bp, synthetic construct
  source 1..22: mol_type = other DNA
ttgtccagctctggtccatgca

SEQ ID NO: 54 - DNA, 23 bp, synthetic construct
  source 1..23: mol_type = other DNA
tgccgactacaagcgaattactg

SEQ ID NO: 55 - DNA, 22 bp, synthetic construct
  source 1..22: mol_type = other DNA
ctgcttgtccagatgacttcgg

SEQ ID NO: 56 - DNA, 23 bp, synthetic construct
  source 1..23: mol_type = other DNA
ctcgttccacatacctcaagtcc

SEQ ID NO: 57 - DNA, 23 bp, synthetic construct
  source 1..23: mol_type = other DNA
ctggaacctttaggatgtgagtg

SEQ ID NO: 58 - DNA, 22 bp, synthetic construct
  source 1..22: mol_type = other DNA
cactgtcaccagcctctggata

SEQ ID NO: 59 - DNA, 23 bp, synthetic construct
  source 1..23: mol_type = other DNA
tcatagccagagatggatacctc

SEQ ID NO: 60 - DNA, 22 bp, synthetic construct
  source 1..22: mol_type = other DNA
acacccagatcgacaactcctg

SEQ ID NO: 61 - DNA, 22 bp, synthetic construct
  source 1..22: mol_type = other DNA
caaacaggtgctgaccatccac

SEQ ID NO: 62 - DNA, 22 bp, synthetic construct
  source 1..22: mol_type = other DNA
tatcctcgctgtggttggactg

SEQ ID NO: 63 - DNA, 24 bp, synthetic construct
  source 1..24: mol_type = other DNA
tagtccaagtaattgtgctagagc

SEQ ID NO: 64 - DNA, 23 bp, synthetic construct
  source 1..23: mol_type = other DNA
cacgccaataactcagtcactgg

SEQ ID NO: 65 - DNA, 22 bp, synthetic construct
  source 1..22: mol_type = other DNA
ttgtggagcaggtcaggttcac

SEQ ID NO: 66 - DNA, 22 bp, synthetic construct
  source 1..22: mol_type = other DNA
tcttcatggtcaccacggctgt

SEQ ID NO: 67 - DNA, 22 bp, synthetic construct
  source 1..22: mol_type = other DNA
acagggagacacactgggcata

SEQ ID NO: 68 - DNA, 22 bp, synthetic construct
  source 1..22: mol_type = other DNA
caccacatacctgcttgtcagc

SEQ ID NO: 69 - DNA, 22 bp, synthetic construct
  source 1..22: mol_type = other DNA
tctccgtctgctctaagaggtg

SEQ ID NO: 70 - DNA, 23 bp, synthetic construct
  source 1..23: mol_type = other DNA
ggctggagtctactatgtcttct

SEQ ID NO: 71 - DNA, 19 bp, synthetic construct
  source 1..19: mol_type = other DNA
acctcggtgaagggagtcc

SEQ ID NO: 72 - DNA, 21 bp, synthetic construct
  source 1..21: mol_type = other DNA
aaactcgcatctactggcaaa

SEQ ID NO: 73 - DNA, 21 bp, synthetic construct
  source 1..21: mol_type = other DNA
ggttcttgtactcgggccata

SEQ ID NO: 74 - DNA, 21 bp, synthetic construct
  source 1..21: mol_type = other DNA
gtgcagtccaggttatcgtgt

SEQ ID NO: 75 - DNA, 21 bp, synthetic construct
  source 1..21: mol_type = other DNA
cacttgcttaggccattgagg

SEQ ID NO: 76 - DNA, 20 bp, synthetic construct
  source 1..20: mol_type = other DNA
ggcagggtcagacttgatcc

SEQ ID NO: 77 - DNA, 22 bp, synthetic construct
  source 1..22: mol_type = other DNA
gtaggtgctgttgtcctctttc

SEQ ID NO: 78 - DNA, 22 bp, synthetic construct
  source 1..22: mol_type = other DNA
tacaaaggcagtgaccatttgg

SEQ ID NO: 79 - DNA, 23 bp, synthetic construct
  source 1..23: mol_type = other DNA
aggtgtaaattccttcgtccaga

SEQ ID NO: 80 - DNA, 23 bp, synthetic construct
  source 1..23: mol_type = other DNA
agatgcaccatccaactgtgtgg

SEQ ID NO: 81 - DNA, 22 bp, synthetic construct
  source 1..22: mol_type = other DNA
aggcagaggattcctacgatgc

SEQ ID NO: 82 - DNA, 22 bp, synthetic construct
  source 1..22: mol_type = other DNA
gcctgatctcatagagtctggc

SEQ ID NO: 83 - DNA, 22 bp, synthetic construct
  source 1..22: mol_type = other DNA
tgcatcccagaactagacgtgc

SEQ ID NO: 84 - DNA, 20 bp, synthetic construct
  source 1..20: mol_type = other DNA
taccctgcccgagtcttctg

SEQ ID NO: 85 - DNA, 21 bp, synthetic construct
  source 1..21: mol_type = other DNA
tggtggtaggtgtaggggttc

SEQ ID NO: 86 - DNA, 19 bp, synthetic construct
  source 1..19: mol_type = other DNA
cgcggatcgtcaacatctc

SEQ ID NO: 87 - DNA, 19 bp, synthetic construct
  source 1..19: mol_type = other DNA
gttcggcggtcactaggtg

SEQ ID NO: 88 - DNA, 20 bp, synthetic construct
  source 1..20: mol_type = other DNA
ccaggccaagattcgagagg

SEQ ID NO: 89 - DNA, 22 bp, synthetic construct
  source 1..22: mol_type = other DNA
ccgatgtgatacctgaagagca

SEQ ID NO: 90 - DNA, 21 bp, synthetic construct
  source 1..21: mol_type = other DNA
agtggctcccaatgcaaacta

SEQ ID NO: 91 - DNA, 21 bp, synthetic construct
  source 1..21: mol_type = other DNA
tatacagccgcacctcaaaag

SEQ ID NO: 92 - DNA, 21 bp, synthetic construct
  source 1..21: mol_type = other DNA
tgccgctatcgaaaatgtctt

SEQ ID NO: 93 - DNA, 20 bp, synthetic construct
  source 1..20: mol_type = other DNA
gggtaggggtgagttgtggt

SEQ ID NO: 94 - DNA, 22 bp, synthetic construct
  source 1..22: mol_type = other DNA
tgcggactacaagcgaatcacg

SEQ ID NO: 95 - DNA, 22 bp, synthetic construct
  source 1..22: mol_type = other DNA
ctcagcttctggataaccctcg

SEQ ID NO: 96 - DNA, 22 bp, synthetic construct
  source 1..22: mol_type = other DNA
acagttccacccgcctcacatt

SEQ ID NO: 97 - DNA, 24 bp, synthetic construct
  source 1..24: mol_type = other DNA
tagaaagaccagtccttgctgaag

SEQ ID NO: 98 - DNA, 22 bp, synthetic construct
  source 1..22: mol_type = other DNA
ggcaatgagcagagtttccgag

SEQ ID NO: 99 - DNA, 22 bp, synthetic construct
  source 1..22: mol_type = other DNA
ccacttcacagccagagatcac

SEQ ID NO: 100 - DNA, 22 bp, synthetic construct
  source 1..22: mol_type = other DNA
tgaggaacctgctcggctacta

SEQ ID NO: 101 - DNA, 22 bp, synthetic construct
  source 1..22: mol_type = other DNA
ggtcttcgttcagggcgatgta

SEQ ID NO: 102 - DNA, 23 bp, synthetic construct
  source 1..23: mol_type = other DNA
ccctatctctgataccgttgtcc

SEQ ID NO: 103 - DNA, 22 bp, synthetic construct
  source 1..22: mol_type = other DNA
tgccgcaactactgtgattcgg

SEQ ID NO: 104 - DNA, 22 bp, synthetic construct
  source 1..22: mol_type = other DNA
ctacagtggctgtgggaaagtc

SEQ ID NO: 105 - DNA, 22 bp, synthetic construct
  source 1..22: mol_type = other DNA
ctcatccgagcgcgagaacttt

SEQ ID NO: 106 - DNA, 22 bp, synthetic construct
  source 1..22: mol_type = other DNA
tctgtggcaaggctttctccag

SEQ ID NO: 107 - DNA, 22 bp, synthetic construct
  source 1..22: mol_type = other DNA
tgcagatgtgccctcaggtttg

SEQ ID NO: 108 - DNA, 19 bp, synthetic construct
  source 1..19: mol_type = other DNA
ccgggtccccaggtaaaga

SEQ ID NO: 109 - DNA, 23 bp, synthetic construct
  source 1..23: mol_type = other DNA
tgtccggtaaaataagaggcttg

SEQ ID NO: 110 - DNA, 21 bp, synthetic construct
  source 1..21: mol_type = other DNA
aggtcggtgtgaacggatttg

SEQ ID NO: 111 - DNA, 23 bp, synthetic construct
  source 1..23: mol_type = other DNA
tgtagaccatgtagttgaggtca

SEQ ID NO: 112 - DNA, 21 bp, synthetic construct
  source 1..21: mol_type = other DNA
ccaatgcaagggctatctcaa

SEQ ID NO: 113 - DNA, 16 bp, synthetic construct
  source 1..16: mol_type = other DNA
caccgtgcctgtgtgc

SEQ ID NO: 114 - DNA, 20 bp, synthetic construct
  source 1..20: mol_type = other DNA
aaaagggagcacacaggcac

SEQ ID NO: 115 - DNA, 20 bp, synthetic construct
  source 1..20: mol_type = other DNA
agtattatcccgcgctgaac

SEQ ID NO: 116 - DNA, 20 bp, synthetic construct
  source 1..20: mol_type = other DNA
agaagttcagcgcgggataa

SEQ ID NO: 117 - DNA, 19 bp, synthetic construct
  source 1..19: mol_type = other DNA
ggctgcggaagcctattct

SEQ ID NO: 118 - DNA, 21 bp, synthetic construct
  source 1..21: mol_type = other DNA
aagatgtagctcgggatggga

SEQ ID NO: 119 - DNA, 21 bp, synthetic construct
  source 1..21: mol_type = other DNA
atcgtggattctgtgacttcc

SEQ ID NO: 120 - DNA, 20 bp, synthetic construct
  source 1..20: mol_type = other DNA
ggaagtcacagaatccacga

SEQ ID NO: 121 - DNA, 20 bp, synthetic construct
  source 1..20: mol_type = other DNA
aaagtcagcagcagacccat

SEQ ID NO: 122 - DNA, 20 bp, synthetic construct
  source 1..20: mol_type = other DNA
gtgcgttcagatgttggctt

SEQ ID NO: 123 - DNA, 22 bp, synthetic construct
  source 1..22: mol_type = other DNA
attttcaccgggaagagtttcg

SEQ ID NO: 124 - DNA, 21 bp, synthetic construct
  source 1..21: mol_type = other DNA
ctaaactgaaagcttccgccg

SEQ ID NO: 125 - DNA, 21 bp, synthetic construct
  source 1..21: mol_type = other DNA
ctctgcccaaggcagcaaatc

SEQ ID NO: 126 - DNA, 20 bp, synthetic construct
  source 1..20: mol_type = other DNA
tgaggggtttcctccaccat

SEQ ID NO: 127 - DNA, 18 bp, synthetic construct
  source 1..18: mol_type = other DNA
ctcggcctcccaaagtgt

SEQ ID NO: 128 - DNA, 18 bp, synthetic construct
  source 1..18: mol_type = other DNA
gccactgtaccccagcct

SEQ ID NO: 129 - DNA, 20 bp, synthetic construct
  source 1..20: mol_type = other DNA
gctcaccagcgttctttcct

SEQ ID NO: 130 - DNA, 23 bp, synthetic construct
  source 1..23: mol_type = other DNA
caaaatgaaaacacagcttttgg

SEQ ID NO: 131 - DNA, 23 bp, synthetic construct
  source 1..23: mol_type = other DNA
ggcaaaacaacacacatttatct

SEQ ID NO: 132 - DNA, 22 bp, synthetic construct
  source 1..22: mol_type = other DNA
tgtgatgcaatttaatacactc

SEQ ID NO: 133 - DNA, 20 bp, synthetic construct
  source 1..20: mol_type = other DNA
acttgagcatatgaggggat

SEQ ID NO: 134 - DNA, 21 bp, synthetic construct
  source 1..21: mol_type = other DNA
attgggactgcatggagcact

SEQ ID NO: 135 - DNA, 22 bp, synthetic construct
  source 1..22: mol_type = other DNA
tttccctgtgtgagtccagaac

SEQ ID NO: 136 - DNA, 22 bp, synthetic construct
  source 1..22: mol_type = other DNA
tacagatccacagtctgggaaa

SEQ ID NO: 137 - DNA, 24 bp, synthetic construct
  source 1..24: mol_type = other DNA
aaaagacctgtgtcaaaacagcat

SEQ ID NO: 138 - DNA, 24 bp, synthetic construct
  source 1..24: mol_type = other DNA
gctgttttgacacaggtcttttac

SEQ ID NO: 139 - DNA, 23 bp, synthetic construct
  source 1..23: mol_type = other DNA
agggattctcatagagaccagtt

SEQ ID NO: 140 - DNA, 22 bp, synthetic construct
  source 1..22: mol_type = other DNA
ctggtttccacagacagatcct

SEQ ID NO: 141 - DNA, 22 bp, synthetic construct
  source 1..22: mol_type = other DNA
tgatccctcttctcctacacca

SEQ ID NO: 142 - DNA, 21 bp, synthetic construct
  source 1..21: mol_type = other DNA
aagagcattgggactgcatgg

SEQ ID NO: 143 - DNA, 21 bp, synthetic construct
  source 1..21: mol_type = other DNA
gtgtgagtccagaacatctcc

SEQ ID NO: 144 - DNA, 20 bp, synthetic construct
  source 1..20: mol_type = other DNA
acccagagtcacagaaccat

SEQ ID NO: 145 - DNA, 24 bp, synthetic construct
  source 1..24: mol_type = other DNA
attccaatctgtgaaagacctgtg

SEQ ID NO: 146 - DNA, 23 bp, synthetic construct
  source 1..23: mol_type = other DNA
accaatattgtgctacctactgt

SEQ ID NO: 147 - DNA, 25 bp, synthetic construct
  source 1..25: mol_type = other DNA
aggagttttctctttaaacctggtg

SEQ ID NO: 148 - DNA, 23 bp, synthetic construct
  source 1..23: mol_type = other DNA
agtccaaggggagaggtaagttt

SEQ ID NO: 149 - DNA, 21 bp, synthetic construct
  source 1..21: mol_type = other DNA
gtccctgaactggactccctg

SEQ ID NO: 150 - DNA, 21 bp, synthetic construct
  source 1..21: mol_type = other DNA
ttggggtctctccctggtttc

SEQ ID NO: 151 - DNA, 22 bp, synthetic construct
  source 1..22: mol_type = other DNA
tcctgatccctcttctcctaca

SEQ ID NO: 152 - DNA, 22 bp, synthetic construct
  source 1..22: mol_type = other DNA
actaaaattgccgagccctttg

SEQ ID NO: 153 - DNA, 23 bp, synthetic construct
  source 1..23: mol_type = other DNA
ccattcattttatggacagggga

SEQ ID NO: 154 - DNA, 20 bp, synthetic construct
  source 1..20: mol_type = other DNA
acagcaaggacatagggagg

SEQ ID NO: 155 - DNA, 22 bp, synthetic construct
  source 1..22: mol_type = other DNA
tgcttcttgtatcccttcagga

SEQ ID NO: 156 - DNA, 20 bp, synthetic construct
  source 1..20: mol_type = other DNA
aggaccttctctgagctgtc

SEQ ID NO: 157 - DNA, 21 bp, synthetic construct
  source 1..21: mol_type = other DNA
gggtttctccattctctgggt

SEQ ID NO: 158 - DNA, 23 bp, synthetic construct
  source 1..23: mol_type = other DNA
acagaagttctccttctgctagg

SEQ ID NO: 159 - DNA, 20 bp, synthetic construct
  source 1..20: mol_type = other DNA
agttaactgctcggacctgc

SEQ ID NO: 160 - DNA, 20 bp, synthetic construct
  source 1..20: mol_type = other DNA
aatgcaggtccgagcagtta

SEQ ID NO: 161 - DNA, 21 bp, synthetic construct
  source 1..21: mol_type = other DNA
tcaggctggaggcacattaag

(19) United States
(12) Patent Application Publication
(10) Pub. No.: US 2025/0032506 A1
(43) Pub. Date: Jan. 30, 2025

(54) GLUCOCORTICOID RECEPTOR BLOCKADE WITH MIFEPRISTONE TO SENSITIZE PANCREATIC CANCER TO IMMUNOTHERAPY

(71) Applicant: BOARD OF REGENTS, THE UNIVERSITY OF TEXAS SYSTEM, Austin, TX (US)

(72) Inventor: Li MA, Houston, TX (US)

(21) Appl. No.: 18/716,052

(22) PCT Filed: Dec. 5, 2022
(86) PCT No.: PCT/US2022/080943

§ 371 Date: Jun. 3, 2024

Related U.S. Application Data
(60) Provisional application No. 63/285,755, filed on Dec. 3, 2021.

(51) Int. Cl. A61K 31/567; A61K 39/00; A61P 35/00; C07K 16/28

(52) U.S. Cl. CPC A61K 31/567; A61P 35/00; C07K 16/2818; C07K 16/2827; A61K 2039/505

(57) ABSTRACT

The present disclosure provides methods of treating a subject having a pancreatic cancer that does not respond to an immune checkpoint inhibitor (ICI) therapy comprising administering to the subject in need thereof a combination therapy comprising one or more glucocorticoid receptor antagonists, or pharmaceutically acceptable salts thereof, and one or more immune checkpoint inhibitor selected from the group consisting of a PD-1 antagonist, a PD-L1 antagonist, a CTLA-4 antagonist, or any combination thereof, thereby treating the subject in need thereof.

### CROSS-REFERENCE TO RELATED APPLICATIONS

This international application claims priority to U.S. Provisional Application No. 63/285,755, filed Dec. 3, 2021, the entire contents of which are hereby incorporated herein by reference.

### REFERENCE TO A SEQUENCE LISTING SUBMITTED ELECTRONICALLY VIA EFS-WEB

This application includes a Sequence Listing submitted electronically via EFS-Web (name: “4443_016PC01_Seqlisting_ST26.xml”; size: 143,194 bytes; and created on: Dec. 5, 2022), which is hereby incorporated by reference in its entirety.

### FIELD OF DISCLOSURE

The present disclosure pertains to the medical field including oncology, especially for the treatment of a subject having pancreatic cancer.

### BACKGROUND

Pancreatic cancer begins in the tissues of the pancreas, with the most common pancreatic cancer beginning in the cells that line the ducts of the pancreas. Pancreatic ductal adenocarcinoma (PDAC) accounts for more than 90% of all pancreatic malignancies and is a leading cause of cancer-related mortality, with a 5-year survival rate of as low as 6% in the United States. (Kamisawa, T., et al. Pancreatic cancer. Lancet 388, 73-85 (2016)). Unfortunately, only a small subset of patients diagnosed with PDAC present with localized and surgically resectable tumors. Chemotherapy and radiotherapy are routinely employed in pancreatic cancer treatment, but nearly all patients relapse eventually and second-line treatment options are poor.

Immune checkpoint blockade (ICB) or immune checkpoint inhibitor (ICI) therapies, such as monoclonal antibodies against PD-1, PD-L1, or CTLA-4, prolong the survival of a subset of patients with certain cancers such as melanoma, non-small cell lung cancer, or renal-cell cancer, among other cancer types. (Hodi, F. S., et al. Improved survival with ipilimumab in patients with metastatic melanoma. N Engl J Med 363, 711-723 (2010); Topalian, S. L., et al. Safety, activity, and immune correlates of anti-PD-1 antibody in cancer. N Engl J Med 366, 2443-2454 (2012); Brahmer, J. R., et al. Safety and activity of anti-PD-L1 antibody in patients with advanced cancer. N Engl J Med 366, 2455-2465 (2012)).

However, except for the <1% of patients with microsatellite instability-high tumors (Le, D. T., et al. Mismatch repair deficiency predicts response of solid tumors to PD-1 blockade. Science 357, 409-413 (2017)), clinical trials targeting immune checkpoint receptors or their cognate ligands have been ineffective in pancreatic cancer. (Brahmer, J. R. et al. Safety and activity of anti-PD-L1 antibody in patients with advanced cancer. N. Engl. J. Med. 366, 2455-2465 (2012); Royal, R. E., et al. Phase 2 trial of single agent Ipilimumab (anti-CTLA-4) for locally advanced or metastatic pancreatic adenocarcinoma. J Immunother 33, 828-833 (2010). Dual ICB therapy using anti-CTLA-4 and anti-PD-L1 antibodies to target non-redundant pathways of T cell inactivation has also been unsuccessful. (O'Reilly, E. M., et al. Durvalumab With or Without Tremelimumab for Patients With Metastatic Pancreatic Ductal Adenocarcinoma: A Phase 2 Randomized Clinical Trial. JAMA Oncol (2019)).

Thus, there is an unmet medical need for interventions that can effectively treat PDAC.

### BRIEF SUMMARY

Provided herein are methods and compositions that address a medical need for interventions that can effectively treat PDAC.

Certain aspects of the present disclosure are directed to a method of treating a subject having a pancreatic cancer that does not respond to an immune checkpoint inhibitor (ICI) therapy comprising administering to the subject in need thereof a combination therapy comprising one or more glucocorticoid receptor antagonists, or pharmaceutically acceptable salts thereof, and one or more immune checkpoint inhibitor selected from the group consisting of a PD-1 antagonist, a PD-L1 antagonist, a CTLA-4 antagonist, or any combination thereof, thereby treating the subject in need thereof. In some aspects, the pancreatic cancer is pancreatic ductal adenocarcinoma.

In some aspects, the present disclosure is directed to a method for sensitizing a pancreatic tumor to an immune checkpoint inhibitor comprising administering to a subject having the pancreatic tumor a combination therapy comprising a combination of one or more glucocorticoid receptor antagonists, or pharmaceutically acceptable salt thereof, and one or more immune checkpoint inhibitors selected from the group consisting of a PD-1 antagonist, PD-L1 antagonist, CTLA-4 antagonist, or any combination thereof, wherein the pancreatic tumor is pancreatic ductal adenocarcinoma.

In some aspects, the pancreatic cancer or pancreatic tumor does not comprise a microsatellite instability-high tumor.

In some aspects, the one or more glucocorticoid receptor antagonists, or pharmaceutically acceptable salt thereof, sensitizes the pancreatic cancer to the one or more immune checkpoint inhibitors.

In some aspects, the one or more glucocorticoid receptor antagonists, or pharmaceutically acceptable salts thereof, are administered before or concomitantly with the one or more immune checkpoint inhibitors. In some aspects, the one or more glucocorticoid receptor antagonist, or pharmaceutically acceptable salt thereof, is administered orally, intranasally, subcutaneously, intramuscularly, intradermally, intravenously, intra-arterially, parenterally, or by catheterization. In some aspects, the one or more glucocorticoid receptor antagonist, or pharmaceutically acceptable salt thereof, is administered orally.

In some aspects, the one or more immune checkpoint inhibitors are administered subcutaneously, intramuscularly, intravenously, intra-arterially, parenterally, or by catheterization. In some aspects, the one or more immune checkpoint inhibitors are administered intravenously.

In some aspects, the subject is administered the combination therapy as the first line of therapy. In some aspects, the subject is administered the combination therapy after the subject has been previously treated for pancreatic cancer. In some aspects, the subject has been previously treated for pancreatic cancer by administration of an immune checkpoint inhibitor not in combination with a glucocorticoid receptor antagonist, or pharmaceutically acceptable salt thereof.

In some aspects, the one or more glucocorticoid receptor antagonist, or pharmaceutically acceptable salt thereof, is administered in a total daily amount of about 100 mg to about 2500 mg. In some aspects, the one or more glucocorticoid receptor antagonist, or pharmaceutically acceptable salt thereof, is administered as a solid oral dosage form. In some aspects, the solid oral dosage form is a capsule or tablet. In some aspects, the glucocorticoid receptor antagonist is in a pharmaceutically acceptable form. In some aspects, the glucocorticoid receptor antagonist is a pharmaceutically acceptable salt form of mifepristone.

In some aspects, the one or more immune checkpoint inhibitors comprises or is the PD-1 antagonist. In some aspects, the one or more immune checkpoint inhibitors comprises or is the PD-L1 antagonist. In some aspects, the one or more immune checkpoint inhibitors comprises or is the CTLA-4 antagonist. In some aspects, the one or more immune checkpoint inhibitors are the PD-1 antagonist and the CTLA-4 antagonist. In some aspects, the one or more immune checkpoint inhibitors are the PD-L1 antagonist and the CTLA-4 antagonist. In some aspects, the one or more checkpoint inhibitors are the PD-1 antagonist, the PD-L1 antagonist, and the CTLA-4 antagonist.

In some aspects, the one or more immune checkpoint inhibitors comprise an antibody.

In some aspects, the one or more PD-1 antagonist is selected from the group consisting of pembrolizumab, nivolumab, cemiplimab, sintilimab, dostarlimab, tisleizumab, torpipalimab, spartalizumab, camrelizumab, lambrolizumab, AMP-224, pidilizumab, or any combinations thereof.

In some aspects, the PD-1 antagonist is pembrolizumab. In some aspects, the pembrolizumab is administered at a single dose of about 100 mg to about 600 mg. In some aspects, the single dose is administered once weekly, once every two weeks, once every three weeks, once every four weeks, once every five weeks, or once every six weeks. In some aspects, the single dose of pembrolizumab is about 200 mg administered once every three weeks. In some aspects, the single dose of pembrolizumab is about 400 mg administered once every six weeks.

In some aspects, the PD-1 antagonist is nivolumab. In some aspects, the nivolumab is administered at a single dose of about 0.5 mg/kg to about 7.5 mg. In some aspects, the nivolumab is administered at a single dose of about 100 mg to about 750 mg. In some aspects, the single dose of nivolumab is administered once weekly, once every two weeks, once every three weeks, once every four weeks, once every five weeks, or once every six weeks. In some aspects, the single dose of nivolumab is about 1 mg/kg administered once every three weeks. In some aspects, the single dose of nivolumab is about 3 mg/kg administered once every two weeks. In some aspects, the single dose of nivolumab is about 3 mg/kg administered once every three weeks. In some aspects, the single dose of nivolumab is about 240 mg administered once every two weeks. In some aspects, the single dose of nivolumab is about 360 mg administered once every three weeks.

In some aspects, the PD-1 antagonist is cemiplimab. In some aspects, the cemiplimab is administered at a single dose of about 100 mg to about 500 mg. In some aspects, the single dose of cemiplimab is administered once weekly, once every two weeks, once every three weeks, once every four weeks, once every five weeks, or once every six weeks. In some aspects, the single dose of cemiplimab is about 350 mg administered once every three weeks.

In some aspects, the PD-1 antagonist is sintilimab. In some aspects, the sintilimab is administered at a single dose of about 25 mg to about 500 mg. In some aspects, the single dose of sintilimab is administered once weekly, once every two weeks, once every three weeks, once every four weeks, once every five weeks, or once every six weeks. In some aspects, the single dose of sintilimab is about 100 mg administered once every three weeks. In some aspects, the single dose of sintilimab is about 200 mg administered once every three weeks.

In some aspects, the PD-1 antagonist is dostarlimab. In some aspects, the dostarlimab is administered at a single dose of about 100 mg to about 1500 mg. In some aspects, the single dose of dostarlimab is administered once weekly, once every two weeks, once every three weeks, once every four weeks, once every five weeks, or once every six weeks. In some aspects, the single dose of dostarlimab is about 500 mg administered once every three weeks. In some aspects, the single dose of dostarlimab is about 1000 mg administered once every six weeks. In some aspects, the single dose of dostarlimab is about 500 mg administered once every three weeks for the first four doses and then about 1000 mg every six weeks.

In some aspects, the PD-1 antagonist is tislelizumab. In some aspects, the tislelizumab is administered at a single dose of about 50 mg to about 500 mg. In some aspects, the single dose of tislelizumab is administered once weekly, once every two weeks, once every three weeks, once every four weeks, once every five weeks, or once every six weeks. In some aspects, the single dose of tislelizumab is about 200 mg administered once every three weeks.

In some aspects, the PD-1 antagonist is toripalimab. In some aspects, the toripalimab is administered at a single dose of about 1 mg/kg to about 7 mg/kg. In some aspects, the single dose of toripalimab is administered once weekly, once every two weeks, once every three weeks, once every four weeks, once every five weeks, or once every six weeks. In some aspects, the single dose of toripalimab is about 3 mg/kg administered once every two weeks.

In some aspects, the PD-1 antagonist is spartalizumab. In some aspects, the spartalizumab is administered at a single dose of about 100 mg to about 700 mg. In some aspects, the single dose of spartalizumab is administered once weekly, once every two weeks, once every three weeks, once every four weeks, once every five weeks, or once every six weeks. In some aspects, the spartalizumab is administered at single dose of about 300 mg every three weeks. In some aspects, the spartalizumab is administered at a single dose of about 400 mg every four weeks.

In some aspects, the PD-1 antagonist is camrelizumab. In some aspects, the camrelizumab is administered at a single dose of about 50 mg to about 500 mg. In some aspects, the single dose of camrelizumab is administered once weekly, once every two weeks, once every three weeks, once every four weeks, once every five weeks, or once every six weeks. In some aspects, the single dose of camrelizumab is about 200 mg administered once every three weeks.

In some aspects, the PD-1 antagonist is lambrolizumab. In some aspects, the lambrolizumab is administered at a single dose of about 0.5 mg/kg to about 15 mg. In some aspects, the single dose of lambrolizumab is administered once weekly, once every two weeks, once every three weeks, once every four weeks, once every five weeks, or once every six weeks.

In some aspects, the PD-1 antagonist is AMP-224. In some aspects, the AMP-224 is administered at a single dose of about 1 mg/kg to about 15 mg/kg. In some aspects, the single dose of AMP-224 is administered once weekly, once every two weeks, once every three weeks, once every four weeks, once every five weeks, or once every six weeks.

In some aspects, the PD-1 antagonist is pidilizumab. In some aspects, the pidilizumab is administered at a single dose of about 0.5 mg/kg to about 5 mg/kg. In some aspects, the single dose of pidilizumab is administered once weekly, once every two weeks, once every three weeks, once every four weeks, once every five weeks, or once every six weeks.

In some aspects, the one or more PD-L1 antagonist is selected from the group consisting of atezolizumab, durvalumab, avelumab, KN035, MDX-1105, MEDI4736, MPDL3280A, BMS-936559, and combinations thereof.

In some aspects, the PD-L1 antagonist is atezolizumab. In some aspects, atezolizumab is administered at single dose of about 500 mg to about 2500 mg. In some aspects, the single dose of atezolizumab is administered once weekly, once every two weeks, once every three weeks, once every four weeks, once every five weeks, or once every six weeks. In some aspects, the single dose of atezolizumab is about 840 mg administered once every two weeks. In some aspects, the single dose of atezolizumab is about 1200 mg administered once every three weeks. In some aspects, the single dose of atezolizumab is about 1680 mg administered once every four weeks.

In some aspects, the PD-L1 antagonist is durvalumab. In some aspects, the durvalumab is administered at a single dose of about 750 mg to about 2500 mg. In some aspects, the single dose of durvalumab is administered once weekly, once every two weeks, once every three weeks, once every four weeks, once every five weeks, or once every six weeks. In some aspects, the single dose of durvalumab is about 1500 mg administered once every three weeks. In some aspects, the single dose of durvalumab is about 1500 mg administered once every four weeks.

In some aspects, the PD-L1 antagonist is avelumab. In some aspects, the avelumab is administered at a single dose of about 100 mg to about 1500 mg. In some aspects, the single dose of avelumab is administered once weekly, once every two weeks, once every three weeks, once every four weeks, once every five weeks, or once every six weeks. In some aspects, the single dose of avelumab is about 800 mg administered once every two weeks.

In some aspects, the PD-L1 antagonist is KN035. In some aspects, the KN035 is administered at a single dose of about 50 mg to about 750 mg. In some aspects, the single dose of KN035 is administered once weekly, once every two weeks, once every three weeks, once every four weeks, once every five weeks, or once every six weeks. In some aspects, the single dose of KN035 is about 300 mg administered once every week.

In some aspects, the PD-L1 antagonist is MEDI4736. In some aspects, the MEDI4736 is administered at a single dose of about 0.1 mg/kg to about 20 mg/kg. In some aspects, the single dose of MEDI4736 is administered once weekly, once every two weeks, once every three weeks, once every four weeks, once every five weeks, or once every six weeks.

In some aspects, the PD-L1 antagonist is MPDL3280A. In some aspects, the MPDL3280A is administered at a single dose of about 750 mg to about 1500 mg. In some aspects, the single dose of MPDL3280A is administered once weekly, once every two weeks, once every three weeks, once every four weeks, once every five weeks, or once every six weeks.

In some aspects, the PD-L1 antagonist is BMS-936559. In some aspects, the BMS-936559 is administered at a single dose of about 0.1 mg/kg to about 10 mg/kg. In some aspects, the single dose of BMS-936559 is administered once weekly, once every two weeks, once every three weeks, once every four weeks, once every five weeks, or once every six weeks.

In some aspects, the one or more CTLA-4 antagonist is selected from the group consisting of ipilimumab, BMS-986218, AGEN1181, tremelimumab, and combinations thereof. In some aspects, the CTLA-4 antagonist is ipilimumab.

In some aspects, the ipilimumab is administered at a single dose of about 0.25 mg/kg to about 15 mg/kg. In some aspects, the single dose of ipilimumab is administered once weekly, once every two weeks, once every three weeks, once every four weeks, once every five weeks, once every six weeks, once every seven weeks, once every eight weeks, once every nine weeks once every ten weeks, once every 11 weeks, or once every 12 weeks. In some aspects, the single dose of ipilimumab is about 1 mg/kg administered once every three weeks. In some aspects, the single dose of ipilimumab is about 3 mg/kg administered once every three weeks. In some aspects, the single dose of ipilimumab is about 10 mg/kg administered once every three weeks. In some aspects, the single dose of ipilimumab is about 10 mg/kg administered once every 12 weeks.

In some aspects, the CTLA-4 antagonist is BMS-986218. In some aspects, the BMS-986218 is administered in a single dose of about 0.5 mg to about 100 mg. In some aspects, the single dose of BMS-986218 is administered once weekly, once every two weeks, once every three weeks, once every four weeks, once every five weeks, once every six weeks, once every seven weeks, once every eight weeks, once every nine weeks once every ten weeks, once every 11 weeks, or once every 12 weeks. In some aspects, the single dose of BMS-986218 is administered once every four weeks.

In some aspects, the CTLA-4 antagonist is AGEN1181. In some aspects, the AGEN1181 is administered at a single dose of about 0.05 mg/kg to about 10 mg/kg. In some aspects, the single dose of AGEN1181 is administered once weekly, once every two weeks, once every three weeks, once every four weeks, once every five weeks, once every six weeks, once every seven weeks, once every eight weeks, once every nine weeks once every ten weeks, once every 11 weeks, or once every 12 weeks. In some aspects, the single dose of AGEN1181 is about 0.1 mg/kg to about 4 mg/kg administered once every three weeks. In some aspects, the single dose of AGEN1181 is about 1 mg/kg to about 4 mg/kg administered once every six weeks.

In some aspects, the CTLA-4 antagonist is tremelimumab. In some aspects, the tremelimumab is administered as a single dose of about 1 mg/kg to about 15 mg/kg. In some aspects, the single dose of tremelimumab is administered once weekly, once every two weeks, once every three weeks, once every four weeks, once every five weeks, once every six weeks, once every seven weeks, once every eight weeks, once every nine weeks once every ten weeks, once every 11 weeks, or once every 12 weeks.

In some aspects, the tumor weight is reduced by at least 60%. In some aspects, the tumor weight is reduced by at least 75%. In some aspects, the tumor weight is reduced by at least 80%.

### BRIEF DESCRIPTION OF DRAWINGS

FIGS. 1A-1M show that glucocorticoid receptor (GR) activates PD-L1 expression and represses MHC-I expression in human pancreatic cancer cells. FIG. 1A provides qPCR analysis of immune inhibitory and immune co-stimulatory genes in SU86.86 cells with or without mifepristone (MIFE, 20 μM, 72 h) treatment. FIG. 1B provides qPCR analysis of genes involved in the MHC-I antigen presentation pathway in SU86.86 cells with or without MIFE (20 μM, 72 h) treatment. FIG. 1C provides immunoblotting of PD-L1, MHC-I, B2M, and GAPDH in SU86.86 cells with or without MIFE treatment with the indicated concentration for 72 h. FIGS. 1D-IF provide representative flow cytometry plots and quantification (by MFI: mean fluorescence intensity) of cell-surface markers in SU86.86 cells with or without MIFE (20 μM, 72 h) treatment (n=3 biological replicates per group). FIG. 1D provides flow cytometry plots and quantification of PD-L1. FIG. 1E provides flow cytometry plots and quantification of MHC-I. FIG. 1F provides flow cytometry plots and quantification of B2M. FIG. 1G provides qPCR analysis of PD-1.1, HLA-A, HLA-B, HLA-C, B2M, and NR3C1 in SU86.86 cells transduced with control shRNA or GR shRNA. FIG. 1H provides immunoblotting of PD-L1, MHC-I, B2M, GR and GAPDH in SU86.86 cells transduced with control shRNA or GR shRNA. FIGS. 1I-1K provide representative flow cytometry plots and quantification (by MFI: mean fluorescence intensity) of cell-surface markers and GPADH in SU86.86 cells transduced with control shRNA or GR shRNA (n=3 biological replicates per group). FIG. 1I provides flow cytometry plots and quantification of PD-L1. FIG. 1J provides flow cytometry plots and quantification of MHC-I. FIG. 1K provides flow cytometry plots and quantification of B2M. FIG. 1L provides a qPCR analysis of PD-L1, HLA-A, HLA-B, HLA-C, and B2M in SU86.86 cells transduced with control shRNA or GR shRNA, with or without dexamethasone (DEX, 100 nM, 8 h) treatment. FIG. 1M provides immunoblotting of PD-L1, MHC-I, B2M, p-GR (Ser211), GR, and GAPDH in SU86.86 cells transduced with control shRNA or GR shRNA, with or without dexamethasone (DEX, 100 nM, 8 h) treatment. FIG. 1N provides normalized luciferase activity of the reporters containing the indicated human PD-L1 gene promoter regions in SU86.86 cells transduced with control shRNA or GR shRNA, with or without IFNγ (10 ng ml⁻¹, 8 h) treatment (n=4 wells per group). FIG. 1O provides normalized luciferase activity of the reporters containing the indicated human HLA-B, HLA-C, and B2M gene promoter regions in SU86.86 cells transduced with control shRNA or GR shRNA (n=4 wells per group). FIG. 1P provides normalized luciferase activity of the reporters containing the human GR-binding elements, PD-L1, HLA-B, HLA-C, and B2M promoter regions in SU86.86 cells with or without DEX (100 nM, 8 h) treatment (n=4 wells per group). Statistical significance in FIGS. 1A, 1B, 1D-1G, 1I-1L and IN-1P was determined by an unpaired 1-test. Error bars are s.e.m.

FIGS. 2A-2S show modulation of PD-L1 and MHC-I by GR is a common regulatory mechanism in human and mouse PDAC cells. FIG. 2A provides immunoblotting of GR, MHC-I, and GAPDH in a panel of human PDAC cell lines. FIGS. 2B-2C provide qPCR analysis of PD-L1, HLA-A, HLA-B, and HLA-C with or without MIFE (20 μM, 72 h) treatment. FIG. 2B provides qPCR analysis of PD-L1, HLA-A, HLA-B, and HLA-C in HPAC cells. FIG. 2C provides qPCR analysis of PD-L1, HLA-A, HLA-B, and HLA-C in BXPC-3 cells. FIGS. 2D-2E provide immunoblotting of PD-L1, MHC-I, and GAPDH with or without MIFE treatment with the indicated doses for 72 h. FIG. 2D provides immunoblotting of PD-L1, MHC-I, and GAPDH in HPAC cells. FIG. 2E provides immunoblotting of PD-L1, MHC-I, and GAPDH in BXPC-3 cells. FIG. 2F provides qPCR analysis of Nr3c1, Pd-l1, H-2k, H-2d, and B2m in HY24409 cells transduced with control shRNA or GR shRNA (left panel) and provides immunoblotting of GR and GAPDH in HY24409 cells transduced with control shRNA or GR shRNA (right panel). FIG. 2G provides qPCR analysis of Pd-l1, H-2k, H-2d, and B2m in HY24409 cells with or without MIFE (20 μM, 48 h) treatment. FIG. 2H provides qPCR analysis of Pd-l1, H-2k, H-2d, and B2m in HY24409 cells treated with DEX (100 nM, 4 h) and MIFE (100 nM, 4 h), alone or in combination. FIG. 2I provides representative flow cytometry plots and quantification (by MFI: mean fluorescence intensity) of cell-surface PD-L1 in HY24409 cells with or without MIFE (20 μM, 48 h) treatment (n=3 biological replicates per group). FIG. 2J provides representative flow cytometry plots and quantification (by MFI: mean fluorescence intensity) of cell-surface MHC-I in HY24409 cells with or without MIFE (20 μM, 48 h) treatment (n=3 biological replicates per group). FIG. 2K provides representative flow cytometry plots and quantification (by MFI: mean fluorescence intensity) of cell-surface B2M in HY24409 cells with or without MIFE (20 μM, 48 h) treatment (n=3 biological replicates per group). The left panel of FIG. 2L provides qPCR analysis of Pd-l1, H-2k, H-2d, B2m, and Nr3cl in HY19636 cells transduced with control shRNA or GR shRNA. The right panel of FIG. 2L provides immunoblotting of GR and GAPDH in HY19636 cells transduced with control shRNA or GR shRNA. FIG. 2M provides representative flow cytometry plots and quantification (by MFI: mean fluorescence intensity) of cell-surface PD-L1 in HY19636 cells transduced with control shRNA or GR shRNA (n=3 biological replicates per group). FIG. 2N provides representative flow cytometry plots and quantification (by MFI: mean fluorescence intensity) of cell-surface MHC-I in HY19636 cells transduced with control shRNA or GR shRNA (n=3 biological replicates per group). FIG. 2O provides representative flow cytometry plots and quantification (by MFI: mean fluorescence intensity) of cell-surface B2M in HY19636 cells transduced with control shRNA or GR shRNA (n=3 biological replicates per group). FIG. 2P provides qPCR analysis of Pd-l1, H-2k, H-2d, and B2m in HY19636 cells with or without MIFE (20 μM, 48 h) treatment. FIG. 2Q provides representative flow cytometry plots and quantification (by MFI: mean fluorescence intensity) of cell-surface PD-L1 in HY19636 cells with or without MIFE (20 μM, 48 h) treatment in HY19636 cells with or without MIFE (20 μM, 48 h) treatment (n=3 biological replicates per group). FIG. 2R provides representative flow cytometry plots and quantification (by MFI: mean fluorescence intensity) of cell-surface MHC-I in HY19636 cells with or without MIFE (20 μM, 48 h) treatment (n=3 biological replicates per group). FIG. 2S provides representative flow cytometry plots and quantification (by MFI: mean fluorescence intensity) of cell-surface B2M in HY19636 cells with or without MIFE (20 μM, 48 h) treatment (n=3 biological replicates per group). Statistical significance in FIGS. 2B-2C and FIGS. 2F-2S was determined by an unpaired t-test. Error bars are s.e.m.

FIGS. 3A-3J show tumor cell-specific GR depletion or pharmacologic GR inhibition suppresses pancreatic tumor growth. FIGS. 3A-3E provide results from C57BL/6 mice bearing orthotopic pancreatic tumors (implanted with 8×10⁴ luciferase labeled HY24409 cells transduced with control shRNA or GR shRNA) that received isotype control (IgG) or anti-CD8 antibody treatment (n=5 mice per group). FIG. 3A provides the study design. MRI: magnetic resonance imaging. FIG. 3B provides representative magnetic resonance images on day 19 after tumor cell implantation. FIG. 3C provides tumor size quantification on day 19 after tumor cell implantation. FIG. 3D provides endpoint images of orthotopic HY24409 tumors expressing either control shRNA or GR shRNA, with or without CD8+ T cell depletion (left image: pancreatic tissue/tumor+spleen; right image: pancreatic tumor with spleen and normal pancreas removed). FIG. 3E provides endpoint tumor weight. FIGS. 3F-3J provide results from C57BL/6 mice bearing orthotopic pancreatic tumors (implanted with 8×10⁴ luciferase-labeled HY24409 cells) that received vehicle or mifepristone (MIFE) and isotype control (IgG) or anti-CD8 antibody treatment (n=5 mice per group). FIG. 3F provides the study design. MRI: magnetic resonance imaging. FIG. 3G provides representative magnetic resonance images on day 17 after tumor cell implantation. FIG. 3H provides tumor size quantification on day 17 after tumor cell implantation. FIG. 3I provides endpoint images of vehicle- and MIFE-treated orthotopic HY24409 tumors, with or without CD8+ T cell depletion (left image: pancreatic tissue/tumor+spleen; right image: pancreatic tumor with spleen and normal pancreas removed). FIG. 3J provides endpoint tumor weight. Statistical significance in FIGS. 3C, 3E, 3H, and 3J was determined by an unpaired t-test. Error bars are s.e.m.

FIGS. 4A-40 show tumor cell-specific GR depletion or pharmacologic GR inhibition promotes antitumor immunity in PDAC. FIG. 4A provides CyTOF-based immune profiling of orthotopic HY24409 tumors expressing either control shRNA or GR shRNA. FIG. 4B provides CyTOF-based immune profiling of vehicle- and MIFE-treated orthotopic HY24409 tumors. Representative viSNE plots were colored by immune cell populations. FIG. 4C provides quantification of CD8+ T cells in orthotopic HY24409 tumors expressing either control shRNA or GR shRNA (n=3-4 mice per group). FIG. 4D provides quantification of granzyme B (GB)+CTLs in orthotopic HY24409 tumors expressing either control shRNA or GR shRNA (n=3-4 mice per group). FIG. 4E provides quantification of CD8+ T cells in vehicle- and MIFE treated orthotopic HY24409 tumors (n=4 mice per group). FIG. 4F provides quantification of granzyme B (GB)+ CTLs in vehicle- and MIFE treated orthotopic HY24409 tumors (n=4 mice per group). FIG. 4G provides multiplex immunofluorescent staining of CD3, CD8, and granzyme B in vehicle- and MIFE-treated orthotopic HY24409 tumors. FIG. 4H provides quantification of CD8 signals. FIG. 4I provides quantification of granzyme B signals (n=5 mice per group). Scale bars, 50 μm. FIG. 4J provides flow cytometric analysis of cell-surface PD-L1 (left panel), MHC-I (H-2K^{b}) (middle panel), and B2M (right panel) levels in cancer cells (gated by ZombieDye-CD45-luciferase+) from orthotopic HY24409 tumors expressing either control shRNA or GR shRNA, with or without CD8+ T cell depletion. MFI: mean fluorescence intensity (n=3-5 mice per group). FIG. 4K provides flow cytometric analysis of cell-surface PD-L1 (left panel), MHC-I (H-2K^{b}) (middle panel), and B2M (right panel) levels in cancer cells (gated by ZombieDye-CD45-luciferase+) from vehicle- and MIFE-treated orthotopic HY24409 tumors, with or without CD8+T cell depletion. MFI: mean fluorescence intensity (n=5 mice per group). FIG. 4L provides qPCR analysis of Pd-l1, H-2k, H-2d, and B2m in vehicle- and MIFE-treated orthotopic HY24409 tumors (n=5 mice per group). FIG. 4M provides qPCR analysis of known GR-activated target genes (Klf9, Snai2, and Fn1) in vehicle- and MIFE-treated orthotopic HY24409 tumors (n=5 mice per group). FIG. 4N provides quantification (by flow cytometry) of PD-1+ (left panel), Tim-3+ (middle panel), and LAG-3+ (right panel) CTLs in vehicle- and MIFE-treated orthotopic HY24409 tumors (n=4 mice per group). FIG. 4O provides quantification (by flow cytometry) of TNFα (right panel), IFNγ (middle panel), and IL-2 (right panel) expression in intratumoral CD8+ T cells from orthotopic HY24409 tumors, after ex vivo phorbol myristate acetate (PMA)/ionomycin stimulation (n=4 mice per group). Statistical significance in FIGS. 4C-4F and 4H-4O was determined by an unpaired t-test. Error bars are s.e.m.

FIGS. 5A-5Q show GR-mediated regulation of MHC-I is required for the anti-tumor effect of GR depletion or inhibition. FIG. 5A provides qPCR analysis of B2m in HY24409 cells transduced with control shRNA or B2M shRNA. FIG. 5B provides representative flow cytometry plots (left panel) and quantification (right panel; by MFI: mean fluorescence intensity) of cell-surface H-2K^{b}/D^{b} in HY24409 cells transduced with GR shRNA and B2M shRNA, alone or in combination (n=3 biological replicates per group). Cells were treated with IFNγ (10 ng ml-1, overnight). FIG. 5C provides representative flow cytometry plots (left panel) and quantification (right panel; by MFI: mean fluorescence intensity) of cell-surface H-2K^{b} in HY24409 cells transduced with GR shRNA and B2M shRNA, alone or in combination. Cells were treated with IFNγ (10 ng ml⁻¹, overnight) (n=3 biological replicates per group). FIG. 5D provides representative flow cytometry plots (left panel) and quantification (right panel; by MFI: mean fluorescence intensity) of cell-surface H-2K^{b}/D^{b} in HY24409 cells transduced with control shRNA or B2M shRNA, with or without mifepristone (MIFE) treatment (20 μM, 48 h). Cells were treated with IFNγ (10 ng ml⁻¹, overnight) (n=3 biological replicates per group). FIG. 5E provides representative flow cytometry plots (left panel) and quantification (right panel; by MFI: mean fluorescence intensity) of cell-surface H-2K^{b} in HY24409 cells transduced with control shRNA or B2M shRNA, with or without mifepristone (MIFE) treatment (20 μM, 48 h). Cells were treated with IFNγ (10 ng ml⁻¹, overnight) (n=3 biological replicates per group). FIG. 5F provides representative flow cytometry plots and quantification (right panel; by MFI: mean fluorescence intensity) of cell-surface MHC-I (H-2K^{b}) in orthotopic pancreatic tumors formed by HY24409 cells transduced with GR shRNA and B2M shRNA, alone or in combination. FIG. 5G provides representative flow cytometry plots (left panel) and quantification (right panel; by MFI: mean fluorescence intensity) of cell-surface MHC-I (H-2K^{b}) in orthotopic pancreatic tumors formed by HY24409 cells transduced with control shRNA or B2M shRNA, with or without mifepristone (MIFE) treatment (n=5 mice per group). FIG. 5H provides quantification of CD8+ T cells in orthotopic HY24409 tumors of the indicated groups (n=5 mice per group). FIG. 5I provides quantification of granzyme B (GB)+ CTLs in orthotopic HY24409 tumors of the indicated groups (n=5 mice per group). FIG. 5J provides quantification of CD8+ T cells in orthotopic HY24409 tumors of the indicated groups (n=5 mice per group) FIG. 5K provides quantification of granzyme B (GB)+ CTLs in orthotopic HY24409 tumors of the indicated groups (n=5 mice per group). FIGS. 5L-5N show data from mice implanted with 8×10⁴ luciferase-labeled HY24409 cells transduced with GR shRNA and B2M shRNA, alone or in combination. FIG. 5L provides endpoint (22 days after inoculation) tumor images of C57BL/6 mice bearing orthotopic pancreatic tumors (left image: pancreatic tissue/tumor+spleen; right image: pancreatic tumor with spleen and normal pancreas removed; n=6 mice per group). FIG. 5M provides endpoint tumor size of C57BL/6 mice bearing orthotopic pancreatic tumors (n=6 mice per group). FIG. 5N provides endpoint tumor weight of C57BL/6 mice bearing orthotopic pancreatic tumors. (n=6 mice per group). FIGS. 5O-5Q show data from mice implanted with 8×10⁴ luciferase-labeled HY24409 cells transduced with control shRNA or B2M shRNA, and treated with vehicle or mifepristone (MIFE) twice every 3 days. FIG. 5O provides endpoint (22 days after inoculation) tumor images of C57BL/6 mice bearing orthotopic pancreatic tumors (left image: pancreatic tissue/tumor+spleen; right image: pancreatic tumor with spleen and normal pancreas removed; n=7 mice per group). FIG. 5P provides endpoint tumor size of C57BL/6 mice bearing orthotopic pancreatic tumors (n=7 mice per group). FIG. 5Q provides endpoint tumor weight of C57BL/6 mice bearing orthotopic pancreatic tumors (n=7 mice per group). Statistical significance in FIGS. 5A-5K, 5M-5N, and 5P-5Q was determined by an unpaired 1-test. Error bars are s.e.m.

FIGS. 6A-6Q show tumor cell-specific GR depletion or pharmacologic GR inhibition sensitizes PDAC to immunotherapy in male mice. FIGS. 6A-6D provide results from C57BL/6 mice bearing orthotopic pancreatic tumors (implanted with 8×10⁴ luciferase labeled HY24409 cells transduced with control shRNA or GR shRNA) that were treated with isotype control (IgG) or dual ICB (anti-PD1 and anti-CTLA-4 monoclonal antibodies; n=6 mice per group). FIG. 6A provides the study design. MRI: magnetic resonance imaging. FIG. 6B provides representative magnetic resonance images on day 19 after tumor cell implantation. FIG. 6C provides tumor size quantification on day 19 after tumor cell implantation. FIG. 6D provides endpoint tumor weight. FIGS. 6E-6H provide results from C57BL/6 mice bearing orthotopic pancreatic tumors (implanted with 4×10⁴ luciferase labeled HY24409 cells) that were treated with mifepristone (MIFE) and dual ICB (anti-PD1 and anti-CTLA-4 monoclonal antibodies), alone or in combination (n=5-7 mice per group). FIG. 6E provides the study design. MRI: magnetic resonance imaging. FIG. 6F provides representative magnetic resonance images on day 24 after tumor cell implantation. FIG. 6G provides tumor size quantification on day 24 after tumor cell implantation. FIG. 6H provides endpoint tumor weight. FIG. 6I provides overall survival curves of HY24409 tumor-bearing C57BL/6 mice treated with mifepristone (MIFE) and dual ICB (anti-PD1 and anti-CTLA-4 monoclonal antibodies), alone or in combination. Statistical significance was determined by the log-rank test. n=5-7 mice per group. FIGS. 6J-6Q provide results from flow cytometric analysis and multiplex immunofluorescent staining of tissues (orthotopic HY24409 tumors or spleens) from C57BL/6 mice treated with mifepristone (MIFE) and dual ICB (anti-PD1 and anti-CTLA-4 monoclonal antibodies), alone or in combination (n=4-5 mice per group). FIG. 6J provides quantification of CD8+ T cells in the spleens by flow cytometry. FIG. 6K provides quantification of CD8+ T cells in pancreatic tumors by flow cytometry. FIG. 6L provides multiplex immunofluorescent staining of CD3, CD8, and granzyme B in the tumors. Scale bars, 50 μm. FIG. 6M provides quantification of CD8 signals. FIG. 6N provides quantification of granzyme B signals. FIG. 6O provides flow cytometric analysis of cell-surface PD-L1 in tumor cells (gated by ZombieDye-CD45-luciferase+). MFI: mean fluorescence intensity. FIG. 6P provides flow cytometric analysis of cell surface MHC-I (H-2K^{b}) in tumor cells (gated by ZombieDye-CD45-luciferase+). MFI: mean fluorescence intensity. FIG. 6Q provides flow cytometric analysis of B2M in tumor cells (gated by ZombieDye-CD45-luciferase+). MFI: mean fluorescence intensity. Statistical significance in FIGS. 6C-6D, 6G-6H, 6J-6K, and 6M-6Q was determined by an unpaired t-test. Error bars are s.e.m.

FIGS. 7A-7J show depletion or inhibition of GR suppresses pancreatic tumor growth and renders sensitivity to immunotherapy in female mice. FIGS. 7A-7E provide results from C57BL/6 mice bearing orthotopic pancreatic tumors (implanted with 4×10⁴ RFP-labeled HY19636 cells) that were treated with mifepristone (MIFE) and dual ICB (anti-PD1 and anti-CTLA-4 monoclonal antibodies), alone or in combination (n=6 mice per group). FIG. 7A provides the study design. FIG. 7B provides endpoint tumor images (left image: pancreatic tissue/tumor+spleen; right image: pancreatic tumor with spleen and normal pancreas removed). FIG. 7C provides endpoint tumor size. FIG. 7D provides endpoint tumor weight. FIG. 7E provides body weight. FIGS. 7F-7I provide results from C57BL/6 mice that were orthotopically implanted with 8×10⁴ RFP-labeled HY19636 cells transduced with control shRNA or GR shRNA (n=6 mice per group). FIG. 7F provides endpoint tumor images (left image: pancreatic tissue/tumor+spleen; right image: pancreatic tumor with spleen and normal pancreas removed). FIG. 7G provides endpoint tumor size. FIG. 7H provides endpoint tumor weight. FIG. 7I provides body weight. FIG. 7J provides immunoblotting of progesterone receptor (PR) and GAPDH in the indicated mouse and human pancreatic cancer cell lines. The MCF-7 cell line was used as a positive control. Statistical significance in FIGS. 7C, 7D, 7G, and 7H was determined by an unpaired t-test. Error bars are s.e.m.

FIGS. 8A-8M show GR correlates with PD-L1 expression, low MHC-I expression, and poor survival in human PDAC. FIG. 8A provides representative immunohistochemical (IHC) staining of GR in the normal pancreatic duct and PDAC. Scale bars, 100 μm (left), 50 μm (middle), and 50 μm (right). FIG. 8B provides quantification of GR-positive and GR-negative cases of PDAC and adjacent normal pancreatic tissue (n=101 patients; statistical significance was determined by Fisher's exact test). FIG. 8C provides GR (encoded by NR3C1) mRNA levels in paired normal pancreatic tissue and PDAC based on the GSE15471 dataset (n=36 patients; statistical significance was determined by a paired t-test). FIG. 8D provides representative IHC staining of the correlation of GR protein levels with PD-L1, MHC-I, and CD8 proteins levels in patients with PDAC. FIG. 8E provides statistical analysis of the correlation of GR (top row) protein levels with PD-L1 (second row), MHC-I (third row), and CD8 (bottom row) proteins levels in patients with PDAC (n=101 patients). FIG. 8F shows the correlation of NR3C1 (encoding GR) mRNA levels with (I) 274 (encoding PD-L1) mRNA levels in PDAC based on TCGA data (n=178 patients). FIG. 8G shows the correlation of NR3C1 (encoding GR) mRNA levels with HLA-A mRNA levels in PDAC based on TCGA data (n=178 patients). FIG. 8H provides Kaplan-Meier curves of overall survival of pancreatic cancer patients stratified by GR protein levels based on IHC (n=101 patients). FIG. 8I provides Kaplan-Meier curves of overall survival of pancreatic patients stratified by GR (encoded by NR3C1) mRNA levels. Data were obtained from the ICGC (International Cancer Genome Consortium) (n=95 patients). Statistical significance was determined by the log-rank test in FIGS. 8H-8I. FIG. 8J provides plasma cortisol levels in healthy volunteers (n=122) and PDAC patients (n=82). Statistical significance was determined by an unpaired t-test. Error bars are s.e.m. FIG. 8K provides correlation of plasma cortisol levels with PD-L1 protein levels in pancreatic tumors (n=32 patients). FIG. 8L shows the correlation of plasma cortisol levels with MHC-I protein levels in pancreatic tumors (n=32 patients). FIG. 8M provides correlation of plasma cortisol levels with GR protein levels in pancreatic tumors (n=32 patients). Statistical significance was determined by the Pearson correlation test in FIGS. 8E-8G and 8K-8M.

FIGS. 9A-9K show GR activates PD-L1 expression and represses MHC-I expression in the human PDAC cell line SW1990. FIG. 9A provides qPCR analysis of immune inhibitory and immune co-stimulatory genes in SW1990 cells with or without mifepristone (MIFE, 20 μM, 72 h) treatment (n=3 technical replicates per group). FIG. 9B provides qPCR analysis of genes involved in the MHC-I antigen presentation pathway in SW1990 cells with or without MIFE (20 μM, 72 h) treatment (n=3 technical replicates per group). FIG. 9C provides immunoblotting of PD-L1, MHC-I, B2M and GAPDH in SW1990 cells with or without MIFE treatment with the indicated doses for 72 h. FIG. 9D provides representative flow cytometry plots (left panel) and quantification (right panel; by MFI: mean fluorescence intensity) of cell-surface PD-L1 in SW1990 cells with or without MIFE (20 μM, 72 h) treatment (n=3 biological replicates per group). FIG. 9E provides representative flow cytometry plots (left panel) and quantification (right panel; by MFI: mean fluorescence intensity) of cell-surface MHC-I in SW 1990 cells with or without MIFE (20 μM, 72 h) treatment (n=3 biological replicates per group). FIG. 9F provides representative flow cytometry plots (left panel) and quantification (right panel; by MFI: mean fluorescence intensity) of cell-surface B2M in SW1990 cells with or without MIFE (20 μM, 72 h) treatment (n=3 biological replicates per group). FIG. 9G provides qPCR analysis of PD-L1, HLA-A, HLA-B, HLA-C, B2M, and NR3C1 in SW1990 cells transduced with control shRNA or GR shRNA. FIG. 9H provides immunoblotting of PD-L1, MHC-I, B2M, GR, and GAPDH in SW1990 cells transduced with control shRNA or GR shRNA. FIG. 9I provides qPCR analysis of PD-1.1, HLA-A, HLA-B, HLA-C, and B2M in SW1990 cells transduced with control shRNA or GR shRNA, with or without dexamethasone (DEX, 100 nM, 3 h) treatment. FIG. 9J provides immunoblotting of PD-L1, MHC-I, B2M, p-GR (Ser211), GR, and GAPDH in SW 1990 cells transduced with control shRNA or GR shRNA, with or without dexamethasone (DEX, 100 nM, 3 h) treatment. FIG. 9K provides normalized luciferase activity of the reporters containing the indicated human PD-L1 gene promoter regions in SW1990 cells transduced with control shRNA or GR shRNA, with or without IFNγ (10 ng ml⁻¹, 8 h) treatment (n=4 wells per group). Statistical significance in FIGS. 9A-9B, 9D-9G, 9I and 9K was determined by an unpaired 1-test. Error bars are s.e.m.

FIGS. 10A-10G show GR is a direct regulator of PD-L1 and MHC-I genes. FIG. 10A provides a schematic representation of human PD-L1, HLA-A, HLA-B, HLA-C, and B2M gene promoter regions and ChIP-qPCR amplicons (boxes indicate that the binding to GR was significantly induced by dexamethasone treatment, which was reversed by co-treatment with mifepristone). FIGS. 10B-10F show data from endogenous GR immunoprecipitated from SU86.86 cells treated with vehicle or dexamethasone (100 nM) with or without mifepristone (100 nM) for 30 min. FIG. 10B provides ChIP-qPCR analysis showing the occupancy of PD-L1 promoters (PD-L1 #2, left panel; PD-L1 #3, right panel) by GR. FIG. 10C provides ChIP-qPCR analysis showing the occupancy of HLA-A promoter (HLA-A #3) by GR. FIG. 10D provides ChIP-qPCR analysis showing the occupancy of HLA-B promoters (HLA-B #1, left panel; HLA-B #3, right panel) by GR. FIG. 10E provides ChIP-qPCR analysis showing the occupancy of HLA-C promoter (HLA-C#5) by GR. FIG. 10F provides ChIP-qPCR analysis showing the occupancy of B2M promoters (B2M #1, top left panel; B2M #2, top right panel; B2M #3, bottom left panel; B2M #4, bottom right panel) by GR. Statistical significance for FIGS. 10B-10F was determined by an unpaired t-test (error bars are s.e.m; n=3 biological replicates per group). FIG. 10G provides immunoblotting of PD-L1, MHC-I, and GAPDH in SU86.86 cells with or without the treatment of mifepristone (20 μM, 48 h), MG132 (10 μM, 6 h), and chloroquine (CQ, 20 μM, 4 h).

FIGS. 11A-11T show GR inhibition or depletion does not affect cell cycle progression or tumor growth in immunodeficient mice. FIGS. 11A-11E provide results from tumor-bearing NSG mice (implanted with 4×10⁴ HY24409 cells) that received vehicle or mifepristone (MIFE) treatment (n=7 mice per group). FIG. 11A provides the study design. FIG. 11B provides tumor growth curves. FIG. 11C provides endpoint tumor images. FIG. 11D provides endpoint tumor weight. FIG. 11E provides body weight. FIGS. 11F-11J provide results from tumor-bearing NSG mice (implanted with 8×10⁴ HY24160 cells) that received vehicle or mifepristone (MIFE) treatment (n=6 mice per group). FIG. 11F provides study design. FIG. 11G provides tumor growth curves. FIG. 11H provides endpoint tumor images. FIG. 11I provides endpoint tumor weight. FIG. 11J provides body weight. FIG. 11K-11N provide results from NSG mice that were implanted with 1×10⁵ HY24409 cells transduced with control shRNA or GR shRNA (n=6 mice per group). FIG. 11K provides tumor growth curves. FIG. 11L provides endpoint tumor images. FIG. 11M provides endpoint tumor weight. FIG. 11N provides body weight. FIG. 11O provides body weight of C57BL/6 mice bearing orthotopic HY24409 tumors expressing either control shRNA or GR shRNA. FIG. 11P provides body weight of C57BL/6 mice bearing vehicle- and MIFE-treated orthotopic HY24409 tumors with or without CD8+ T cell depletion (n=5 mice per group). FIG. 11Q provides cell-cycle profile histograms in HY24409 cells transduced with control shRNA (left panel) or GR shRNA (right panel). Cells were collected from unsynchronized (Uns) conditions and at different time points after release from double thymidine block (n=3 biological replicates per group). FIG. 11R provides quantitative analysis of the cell-cycle distribution in HY24409 cells transduced with control shRNA (left panel) or GR shRNA (right panel). Cells were collected from unsynchronized (Uns) conditions and at different time points after release from double thymidine block (n=3 biological replicates per group). FIG. 11S provides cell-cycle profile histograms in HY24409 cells pretreated with vehicle (left panel) or mifepristone (right panel; 20 μM, 48 h), followed by double thymidine block and release (n=3 biological replicates per group). FIG. 11T provides quantitative analysis of the cell-cycle distribution in HY24409 cells pretreated with vehicle (left panel) or mifepristone (right panel; 20 μM, 48 h), followed by double thymidine block and release (n=3 biological replicates per group). Statistical significance was determined by two-way analysis of variance in FIGS. 11B, 11G, and 11K. Statistical significance was determined by an unpaired t-test in FIGS. 11D, 11I, and 11M. Error bars are s.e.m.

FIGS. 12A-12F show the effects of GR depletion or inhibition on mice bearing the KPC line HY24409. FIG. 12A provides quantification of tumor-infiltrating immune cell populations (top row panels from left to right: CD4+ T cells, Treg cells, NK cells; bottom row panels from left to right: B cells, Dendritic cells, Macrophages, granulocytic myeloid-derived suppressor cells (Gr-MDSCs)) in orthotopic HY24409 tumors expressing either control shRNA or GR shRNA (n=3-4 mice per group). FIG. 12B provides quantification of tumor-infiltrating immune cell populations (top row panels from left to right: CD4+ T cells, Treg cells, NK cells; bottom row panels from left to right: B cells, Dendritic cells, Macrophages, granulocytic myeloid-derived suppressor cells (Gr-MDSCs)) in vehicle and mifepristone (MIFE)-treated orthotopic HY24409 tumors (n=4 mice per group). Statistical significance in FIGS. 12A-12B was determined by an unpaired t-test. Error bars are s.e.m. FIG. 12C provides representative flow cytometry plots (pre-gated on T cells by CD45+CD3+) showing the relative abundance of CD8+T cells in HY24409 tumor-bearing mice that received isotype control (IgG; left two panels) or anti-CD8 (right panels) antibody treatment. FIG. 12C flow cytometry was performed on tumor samples (upper panels) and blood samples (lower panels). FIG. 12D provides representative flow cytometry plots of cell-surface PD-L1 (left panel), MHC-I (H-2K^{b}) (middle panel), and B2M (right panel) levels in cancer cells (gated by ZombieDye-CD45-luciferase+) from orthotopic HY24409 tumors expressing either control shRNA or GR shRNA, with or without CD8+ T cell depletion. FIG. 12E provides representative flow cytometry plots of cell-surface PD-L1 (left panel), MHC-I (H-2K^{b}) (middle panel), and B2M (right panel) levels in cancer cells (gated by ZombieDye-CD45-luciferase+) from vehicle- and MIFE-treated orthotopic HY24409 tumors, with or without CD8+ T cell depletion. FIG. 12F provides chemokine arrays of the conditioned medium of HY24409 cells with (top panels) or without MIFE (lower panels; 20 μM, 48 h) treatment.

FIGS. 13A-13O show the effects of GR inhibition on anti-tumor immunity in the KPC line HY24160. FIGS. 13A-13O show results from C57BL/6 mice bearing orthotopic pancreatic tumors (implanted with 4×10⁴ HY24160 cells) that received vehicle or mifepristone (MIFE) and isotype control (IgG) or anti-CD8 antibody treatment (n=5-6 mice per group). FIG. 13A provides the study design. FIG. 13B provides endpoint tumor images (left images: pancreatic tissue/tumor+spleen; right images: pancreatic tumor with spleen and normal pancreas removed) for isotype control (top panels) or CD8 antibody treatment (bottom panels) with MIFE. FIG. 13C provides endpoint tumor size for isotype control (left panel) or CD8 antibody treatment (right panel) with MIFE. FIG. 13D provides endpoint tumor weight for isotype control (left panel) or CD8 antibody treatment (right panel) with MIFE. FIG. 13E provides body weight for isotype control (left panel) or CD8 antibody treatment (right panel) with MIFE. FIG. 13F provides immune profiling of vehicle (left panel) and MIFE (right panel) treated orthotopic HY24160 tumors by CyTOF. Representative viSNE plots were colored by immune cell populations. FIG. 13G provides CD8+ T cell quantification. FIG. 13H provides quantification of tumor-infiltrating immune cell populations (top row panels from left to right: CD4+ T cells, Treg cells, NK cells; bottom row panels from left to right: B cells, Dendritic cells, Macrophages, granulocytic myeloid-derived suppressor cells (Gr-MDSCs)) in vehicle- and MIFE-treated orthotopic HY24160 tumors. FIG. 13I provides multiplex immunofluorescent staining of CD3, CD8, and granzyme B in vehicle (top panels) and MIFE treated (bottom panels) orthotopic HY24160 tumors (scale bars, 50 μm). FIG. 13J provides quantification of CD8 signals. FIG. 13K provides quantification of granzyme B signals. FIG. 13L provides flow cytometric analysis of cell-surface PD-L1 (left panel) MHC-I (H-2K^{b}; middle panel) and B2M (right panel) levels in tumor cells collected from mice treated with isotype control IgG (MFI: mean fluorescence intensity). FIG. 13M provides flow cytometric analysis of PD-L1 (left panel) MHC-I (H-2K^{b}, middle panel) and B2M (right panel) levels in tumor cells collected from mice treated with anti-CD8 antibody (MFI: mean fluorescence intensity). FIG. 13N provides qPCR analysis of Pd-l1, H-2k, H-2d, and B2m in vehicle- and MIFE-treated orthotopic HY24160 tumors. FIG. 13O provides qPCR analysis of known GR-activated target genes (Klf9, Snai2, and Fn1) in vehicle- and MIFE-treated orthotopic HY24160 tumors. Statistical significance in FIGS. 13C, 13D, 13G, 13H, and 13J-13O was determined by an unpaired t-test. Error bars are s.e.m.

FIGS. 14A-14D show the effect of GR depletion or inhibition on the immunotherapeutic response of the KPC line HY24409 in C57BL/6 mice. FIG. 14A provides endpoint images of orthotopic HY24409 tumors from C57BL/6 mice treated expressing either control shRNA or GR shRNA, with or without dual ICB (anti-PD1 and anti-CTLA-4 monoclonal antibodies) treatment (left image: pancreatic tissue/tumor+spleen; right image: pancreatic tumor with spleen and normal pancreas removed; n=6 mice per group). FIG. 14B provides body weight of the mice from experiments in FIG. 14A. FIG. 14C provides endpoint images of orthotopic HY24409 tumors from C57BL/6 mice treated with mifepristone (MIFE) and dual ICB (anti-PD1 and anti-CTLA-4 monoclonal antibodies), alone or in combination (left image: pancreatic tissue/tumor+spleen; right image: pancreatic tumor with spleen and normal pancreas removed; n=5-7 mice per group). FIG. 14D provides body weight of the mice from experiments in FIG. 14C.

FIG. 15 provides a workflow of gating strategies for flow cytometry analysis.

### DETAILED DESCRIPTION OF THE DISCLOSURE

Non-limiting examples of the various aspects are shown in the present disclosure.

### Definitions

So that the present disclosure can be more readily understood, certain terms are first defined. Additional definitions are set forth throughout the detailed disclosure.

It is to be noted that the term “a” or “an” entity refers to one or more of that entity; for example, “a nucleic acid sequence,” is understood to represent one or more nucleic acid sequences, unless stated otherwise. As such, the terms “a” (or “an”), “one or more,” and “at least one” can be used interchangeably herein.

Furthermore, “and/or”, where used herein, is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The term “subject” refers to an animal, including, but not limited to, a primate (e.g., human), cow, sheep, goat, horse, dog, cat, rabbit, rat, or mouse. The terms “subject” and “patient” are used interchangeably herein in reference, for example, to a mammalian subject, such as a human subject.

As used herein, the term “a subject in need of treatment” refers to an individual or subject that has been diagnosed with a disease or disorder, e.g., a cancer, a tumor, or a cell proliferative disorder

As used herein, the terms “treat,” “treated,” and “treating” mean both therapeutic treatment and prophylactic or preventative measures wherein the object is to prevent or slow down (lessen) an undesired physiological condition, disorder, or disease, or obtain beneficial or desired clinical results. Thus, those in need of treatment include those already diagnosed with or suspected of having the disorder. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment. In the context of cancer, the term “treating” includes, but is not limited to, inhibiting growth of cancer cells, inhibiting replication of cancer cells, lessening of overall tumor burden, and delaying, halting, or slowing tumor growth, progression, or metastasis. As used herein, the term “treating cancer” is not intended to be an absolute term. In some aspects, the methods of treating cancer can cause a cancer to go into remission, or prevent growth in size or cell number of cancer cells. In some circumstances, treatment leads to an improved prognosis.

As used herein, the terms “cancer” and “tumor” refer to or describe the physiological condition in mammals in which a population of cells are characterized by unregulated cell growth. The terms can encompass solid and hematological/lymphatic cancers. In some aspects, the cancer or tumor can be metastatic.

As used herein, the term “does not respond to an immune checkpoint inhibitor (ICI) therapy” refers to a type of cancer or tumor that either has not decreased or has only a clinically insignificant decrease in tumor size after treatment with ICI therapy or is not likely to respond to ICI therapy. See Le, D. T., et al. Mismatch repair deficiency predicts response of solid tumors to PD-1 blockade. Science 357, 409-413 (2017); Hu, Z, et al. Evaluating Mismatch Repair Deficiency in Pancreatic Adenocarcinoma: Challenges and Recommendations. Clin. Cancer Res. 24 (6): 1326-1336 (2018); Marabelle et al. Efficacy of Pembrolizumab in Patients with Noncolorectal High Microsatellite Instability/Mismatch Repair-Deficient Cancer: Results from the Phase II KEYNOTE-158 Study. J. Clin. Oncol. 38 (1): 1-10 (2019). Whether the cancer or tumor is not likely to respond to ICI therapy can be determined by known methods, for example by identifying a subject's genotype or phenotype as being a type not likely to respond to an ICI therapy. Such a genotype or phenotype includes, but is not limited to, those not having high microsatellite instability.

As used herein, the term “administration”, “administer” or “administering” refers to delivery of the therapeutic agent(s) to a subject or desired site of biological action. Administration techniques that can be employed with the agents and methods described herein are found in e.g., Goodman and Gilman, The Pharmacological Basis of Therapeutics, current ed.; Pergamon; and Remington's, Pharmaceutical Sciences (current edition), Mack Publishing Co., Easton, Pa. Administration of two or more therapeutic agents can include simultaneous (concurrent or concomitantly) or consecutive administration in any order, and can be by the same or different route of administration. Administration to an animal subject (e.g., to a human) can be by any appropriate route.

The term “combination therapy”, as used herein, can include a fixed combination in one dosage unit form, separate dosage units, or a kit of parts or instructions for the combined administration where the two or more therapeutic agents can be administered independently at the same time or separately within time intervals.

A “therapeutically effective amount” of a substance can vary according to factors such as the disease state, age, sex, and weight of the individual, and the ability of the substance to elicit a desired response in the individual. A therapeutically effective amount is also one in which any toxic or detrimental effects of the substance are outweighed by the therapeutically beneficial effects. A therapeutically effective amount can be delivered in one or more administrations (e.g., one or more doses). A therapeutically effective amount refers to an amount effective, at dosages and for periods of time necessary, to achieve the desired therapeutic effect.

As used herein, the term “PD-1” refers to Programmed Cell Death Protein 1 (also known as CD279), a cell surface membrane protein of the immunoglobulin superfamily. PD-1 is expressed by B cells, T cells and NK cells. The major role of PD-1 is to limit the activity of T cells in peripheral tissues during inflammation in response to infection, as well as to limit autoimmunity. PD-1 expression is induced on activated T cells and binding of PD-1 to one of its endogenous ligands acts to inhibit T cell activation by inhibiting stimulatory kinases. PD-1 also acts to inhibit the TCR “stop signal”. PD-1 is highly expressed on Treg cells (regulatory T cells) and may increase their proliferation in the presence of ligand.

As used herein, the term “PD-L1” refers to Programmed Cell Death 1 ligand 1 (also known as CD274 and B7-H1), a ligand for PD-1. PD-L1 is found on activated T cells, B cells, myeloid cells, macrophages, and tumor cells. Although there are two endogenous ligands for PD-1, PD-L1 and PD-L2, anti-tumor therapies have focused on anti-PD-L1. The complex of PD-1 and PD-L1 inhibits proliferation of CD8+ T cells and reduces the immune response.

As used herein, the term “CTLA4” refers to Cytotoxic T-lymphocyte antigen 4 (also known as CD152), a member of the immunoglobulin superfamily that is expressed exclusively on T cells. CTLA4 acts to inhibit T cell activation and is reported to inhibit helper T cell activity and enhance regulatory T cell immunosuppressive activity. Although the precise mechanism of action of CTL4-A remains under investigation, it has been suggested that it inhibits T cell activation by outcompeting CD28 in binding to CD80 and CD86 on antigen presenting cells, as well as actively delivering inhibitor signals to the T cell.

As used herein, the term “immune checkpoint inhibitor” refers to any molecule, including, but not limited to, antibodies and small molecules, that block the immunosuppression pathway induced by one or more checkpoint proteins.

As used herein, the term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, chimeric antibodies, humanized antibodies, human antibodies, fusion proteins comprising an antibody, and any other modified immunoglobulin molecule so long as the antibodies exhibit the desired biological activity. An antibody can be of any of the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

As used herein, the term “glucocorticoid receptor” (“GR”) refers to a family of intracellular receptors which specifically bind to cortisol and/or cortisol analogs. The glucocorticoid receptor is also referred to as the cortisol receptor. The term includes isoforms of GR, recombinant GR and mutated GR.

The term “antagonist” as used herein refers to any molecule that partially or fully blocks, inhibits, or neutralizes a biological activity of a native polypeptide disclosed herein. Suitable antagonist molecules specifically include antagonist antibodies or antibody fragments, fragments or amino acid sequence variants of native polypeptides, peptides or proteins. In some embodiments, inhibition in the presence of the antagonist is observed in a dose-dependent manner. In some embodiments, the measured signal (e.g., biological activity) is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% lower than the signal measured with a negative control under comparable conditions.

The term “pharmaceutically-acceptable salts” refers to the relatively non-toxic, inorganic and organic acid addition salts, e.g., of a glucocorticoid receptor antagonist disclosed herein. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

The pharmaceutically acceptable salts of the compounds disclosed herein include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isothionic, and the like.

In certain aspects, the compounds of the present invention may contain one or more acidic functional groups and, thus, are capable of forming pharmaceutically acceptable salts with pharmaceutically acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, e.g., Berge et al., supra).

### Methods of Treatment

In one aspect, the present disclosure is directed to a method of treating a subject having a pancreatic cancer or a pancreatic tumor that does not respond to an immune checkpoint inhibitor (ICI) therapy comprising administering to the subject in need thereof a combination therapy comprising one or more glucocorticoid receptor antagonists, or pharmaceutically acceptable salts thereof, and one or more immune checkpoint inhibitor selected from the group consisting of a PD-1 antagonist, a PD-L1 antagonist, a CTLA-4 antagonist, or any combination thereof, thereby treating the subject in need thereof. In some aspects, the pancreatic cancer is pancreatic ductal adenocarcinoma.

In another aspect, the present disclosure provides a method for sensitizing a pancreatic tumor to an immune checkpoint inhibitor comprising administering to a subject having the pancreatic tumor a combination therapy comprising a combination of one or more glucocorticoid receptor antagonists, or pharmaceutically acceptable salt thereof, and one or more immune checkpoint inhibitors selected from the group consisting of a PD-1 antagonist, PD-L1 antagonist, CTLA-4 antagonist, or any combination thereof, wherein the pancreatic tumor is pancreatic ductal adenocarcinoma.

In some aspects, the tumor weight is reduced by at least 60%. In some aspects, the tumor weight is reduced by at least 75%. In some aspects, the tumor weight is reduced by at least 80%. In some aspects, the tumor weight is reduced by at least 85%.

It was previously shown that pancreatic cancer is highly resistant to ICB therapy. Even targeting multiple immune checkpoints has failed in clinical trials (Leinwand, J. & Miller, G. Regulation and modulation of antitumor immunity in pancreatic cancer. Nat. Immunol. 21, 1152-1159 (2020)). Similarly, female mice with orthotopic implantation of the female KPC line HY15549 do not respond to ICB by anti-CTLA-4 and anti-PD-1 treatment, even when used in combination. (Yamamoto, K. et al. Autophagy promotes immune evasion of pancreatic cancer by degrading MHC-I. Nature 581, 100-105 (2020)). In some aspects, the combination therapy methods disclosed herein are able to overcome ICB therapy resistance in pancreatic cancer.

In some aspects, the one or more glucocorticoid receptor antagonists, or pharmaceutically acceptable salts thereof, sensitizes the pancreatic cancer to the one or more immune checkpoint inhibitors.

In some aspects, the pancreatic cancer or pancreatic tumor does not comprise a microsatellite instability-high tumor.

In some aspects, the glucocorticoid receptor antagonist is in a pharmaceutically acceptable form. In some aspects, the glucocorticoid receptor antagonist is a pharmaceutically acceptable salt form of mifepristone. In some aspects, the glucocorticoid receptor antagonist is in free base form. In some aspects, the glucocorticoid receptor antagonist is mifepristone free base.

In some aspects, the one or more glucocorticoid receptor antagonist, or pharmaceutically acceptable salt thereof, is administered orally, intranasally, subcutaneously, intramuscularly, intradermally, intravenously, intra-arterially, parenterally, or by catheterization. In some aspects, the one or more glucocorticoid receptor antagonist, or pharmaceutically acceptable salt thereof, is administered orally. In some aspects, the one or more glucocorticoid receptor antagonist (e.g., mifepristone), is administered as a solid oral dosage form, such as a tablet or capsule.

In some aspects, the one or more immune checkpoint inhibitors are administered subcutaneously, intramuscularly, intravenously, intra-arterially, parenterally, or by catheterization. In some aspects, the one or more immune checkpoint inhibitors are administered intravenously.

### Dosing Regimens

In some aspects, the one or more glucocorticoid receptor antagonists, or pharmaceutically acceptable salts thereof, are administered before or concomitantly with the one or more immune checkpoint inhibitors. In some aspects, the one or more glucocorticoid receptor antagonists, or pharmaceutically acceptable salts thereof, are administered at least one day before the one or more immune checkpoint inhibitors. In some aspects, the one or more glucocorticoid receptor antagonists, or pharmaceutically acceptable salts thereof, are administered at least two days before the one or more immune checkpoint inhibitors. In some aspects, the one or more glucocorticoid receptor antagonists, or pharmaceutically acceptable salts thereof, are administered at least one week before the one or more immune checkpoint inhibitors. If two or more glucocorticoid receptor antagonists (i.e., mifepristone and a different glucocorticoid receptor antagonist), the two antagonists can be administered simultaneously or at different times.

In some aspects, the subject is administered the combination therapy as the first line of therapy. In such cases, the subject can be identified (e.g., determined to have the presence or absence of a particular genotype or phenotype) as having pancreatic cancer that will not respond to immune checkpoint inhibitor therapy without the glucocorticoid receptor antagonist.

In some aspects, the subject is administered the combination therapy after the subject has been previously treated for pancreatic cancer. Examples of such previous treatment include, but are not limited to chemotherapy, surgery, and radiation therapy. In some aspects, the subject has been previously treated for pancreatic cancer by administration of an immune checkpoint inhibitor not in combination with a glucocorticoid receptor antagonist, or pharmaceutically acceptable salt thereof.

In some aspects, the one or more glucocorticoid receptor antagonist, or pharmaceutically acceptable salt thereof, is administered in a therapeutically effective amount. In some aspects, the one or more glucocorticoid receptor antagonist, or pharmaceutically acceptable salt thereof, is administered in a total daily amount of about 100 mg to about 2500 mg, about 200 mg to about 2500 mg, about 300 mg to about 2500 mg, about 400 mg to about 2500 mg, about 500 mg to about 2500 mg, about 600 mg to about 2500 mg, about 700 mg to about 2500 mg, about 800 mg to about 2500 mg, about 800 mg to about 2500 mg, about 900 mg to about 2500 mg, about 1000 mg to about 2500 mg, about 1100 mg to about 2500 mg, about 1200 mg to about 2500 mg, about 100 mg to about 2400 mg, about 100 mg to about 2300 mg, about 100 mg to about 2200 mg, about 100 mg to about 2100 mg, about 100 mg to about 2000 mg, about 100 mg to about 1900 mg, about 100 mg to about 1800 mg, about 100 mg, to about 1700 mg, about 100 mg to about 1600 mg, about 100 mg to about 1500 mg, about 100 mg to about 1400 mg, about 100 mg to about 1300 mg, about 100 mg to about 1200 mg, about 100 mg to about 1100 mg, about 100 mg to about 1000 mg, about 200 mg to about 2500 mg, about 200 mg to about 2400 mg, about 200 mg to about 2300 mg, about 200 mg to about 2200 mg, about 200 mg to about 2100 mg, about 200 mg to about 2000 mg, about 200 mg to about 1900 mg, about 200 mg to about 1800 mg, about 200 mg to about 1700 mg, about 200 mg to about 1600 mg, about 200 mg to about 1500 mg, about 200 mg to about 1400 mg, about 200 mg to about 1300 mg, about 200 mg to about 1200 mg, about 200 mg to about 1100 mg, about 200 mg to about 1000 mg, about 300 mg to about 2500 mg, about 300 mg to about 2400 mg, about 300 mg to about 2300 mg, about 300 mg to about 2200 mg, about 300 mg to about 2100 mg, about 300 mg to about 2000 mg, about 300 mg to about 1900 mg, about 300 mg to about 1800 mg, about 300 mg to about 1700 mg, about 300 mg to about 1600 mg, about 300 mg to about 1500 mg, about 300 mg to about 1400 mg, about 300 mg to about 1300 mg, about 300 mg to about 1200 mg, about 300 mg to about 1100 mg, about 300 mg to about 1000 mg, about 400 mg to about 2500 mg, about 400 mg to about 2400 mg, about 400 mg to about 2300 mg, about 400 mg to about 2200 mg, about 400 mg to about 2100 mg, about 400 mg to about 2000 mg, about 400 mg to about 1900 mg, about 400 mg to about 1800 mg, about 400 mg to about 1700 mg, about 400 mg to about 1600 mg, about 400 mg to about 1500 mg, about 400 mg to about 1400 mg, about 400 mg to about 1300 mg, about 400 mg to about 1200 mg, about 400 mg to about 1100 mg, about 400 mg to about 1000 mg, about 500 mg to about 2500 mg, about 500 mg to about 2400 mg, about 500 mg to about 2300 mg, about 500 mg to about 2200 mg, about 500 mg to about 2100 mg, about 500 mg to about 2000 mg, about 500 mg to about 1900 mg, about 500 mg to about 1800 mg, about 500 mg to about 1700 mg, about 500 mg to about 1600 mg, about 500 mg to about 1500 mg, about 500 mg to about 1400 mg, about 500 mg to about 1300 mg, about 500 mg to about 1200 mg, about 500 mg to about 1100 mg, about 500 mg to about 1000 mg, about 600 mg to about 2500 mg, about 600 mg to about 2400 mg, about 600 mg to about 2300 mg, about 600 mg to about 2200 mg, about 600 mg to about 2100 mg, about 600 mg to about 2000 mg, about 600 mg to about 1900 mg, about 600 mg to about 1800 mg, about 600 mg to about 1700 mg, about 600 mg to about 1600 mg, about 600 mg to about 1500 mg, about 600 mg to about 1400 mg, about 600 mg to about 1300 mg, about 600 mg to about 1200 mg, about 600 mg to about 1100 mg about 300 mg to about 2500 mg, about 700 mg to about 2400 mg, about 700 mg to about 2300 mg, about 700 mg to about 2200 mg, about 700 mg to about 2100 mg, about 700 mg to about 2000 mg, about 700 mg to about 1900 mg, about 700 mg to about 1800 mg, about 700 mg to about 1700 mg, about 700 mg to about 1600 mg, about 700 mg to about 1500 mg, about 700 mg to about 1400 mg, about 700 mg to about 1300 mg, about 700 mg to about 1200 mg, about 300 mg to about 2500 mg, about 300 mg to about 2400 mg, about 800 mg to about 2300 mg, about 800 mg to about 2200 mg, about 800 mg to about 2100 mg, about 800 mg to about 2000 mg, about 800 mg to about 1900 mg, about 800 mg to about 1800 mg, about 800 mg to about 1700 mg, about 800 mg to about 1600 mg, about 800 mg to about 1500 mg, about 800 mg to about 1400 mg, about 800 mg to about 1300 mg, about 900 mg to about 2500 mg, about 900 mg to about 2400 mg, about 900 mg to about 2300 mg, about 900 mg to about 2200 mg, about 900 mg to about 2100 mg, about 900 mg to about 2000 mg, about 900 mg to about 1900 mg, about 900 mg to about 1800 mg, about 900 mg to about 1700 mg, about 900 mg to about 1600 mg, about 900 mg to about 1500 mg, about 900 mg to about 1400 mg, about 1000 mg to about 2500 mg, about 1000 mg to about 2400 mg, about 1000 mg to about 2300 mg, about 1000 mg to about 2200 mg, about 1000 mg to about 2100 mg, about 1000 mg to about 2000 mg, about 1000 mg to about 1900 mg, about 1000 mg to about 1800 mg, about 1000 mg to about 1700 mg, about 1000 mg to about 1600 mg, about 1000 mg to about 1500 mg, about 1100 mg to about 2500 mg, about 1100 mg to about 2400 mg, about 1100 mg to about 2300 mg, about 1100 mg to about 2200 mg, about 1100 mg to about 2100 mg, about 1100 mg to about 2000 mg, about 1100 mg to about 1900 mg, about 1100 mg to about 1800 mg, about 1100 mg to about 1700 mg, about 1100 mg to about 1600 mg, about 1200 mg to about 2500 mg, about 1200 mg to about 2400 mg, about 1200 mg to about 2300 mg, about 1200 mg to about 2200 mg, about 1200 mg to about 2100 mg, about 1200 mg to about 2000 mg, about 1200 mg to about 1900 mg, about 1200 mg to about 1800 mg, about 1200 mg to about 1700 mg, about 1300 mg to about 2500 mg, about 1300 mg to about 2400 mg, about 1300 mg to about 2300 mg, about 1300 mg to about 2200 mg, about 1300 mg to about 2100 mg, about 1300 mg to about 2000 mg, about 1300 mg to about 1900 mg, about 1300 mg to about 1800 mg, about 1400 mg to about 2500 mg, about 1400 mg to about 2400 mg, about 1400 mg to about 2300 mg, about 1400 mg to about 2200 mg, about 1400 mg to about 2100 mg, about 1400 mg to about 2000 mg, about 1400 mg to about 1900 mg, about 1500 mg to about 2500 mg, about 1500 mg to about 2400 mg, about 1500 mg to about 2300 mg, about 1500 mg to about 2200 mg, about 1500 mg to about 2100 mg, about 1500 mg to about 2000 mg, about 1600 mg to about 2500 mg, about 1600 mg to about 2400 mg, about 1600 mg to about 2300 mg, about 1600 mg to about 2200 mg, or about 1600 mg to about 2100 mg.

In some aspects, the one or more glucocorticoid receptor antagonist, or pharmaceutically acceptable salt thereof, is administered in a total daily amount of about 100 mg, about 110 mg, about 115 mg, about 120 mg, about 125 mg, about 130 mg, about 135 mg, about 140 mg, about 145 mg, about 150 mg, about 160 mg, about 170 mg, about 175 mg, about 180 mg, about 190 mg, about 200 mg, about 225 mg, about 250 mg, about 275 mg, about 300 mg, about 325 mg, about 350 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2100 mg, about 2200 mg, about 2300 mg, about 2400 mg, or about 2500 mg.

In some aspects, the one or more glucocorticoid receptor antagonist, or pharmaceutically acceptable salt thereof, is mifepristone that is administered in a therapeutically effective amount. In some aspects, the one or more glucocorticoid receptor antagonist, or pharmaceutically acceptable salt thereof, is mifepristone that is administered in a total daily amount of about 100 mg to about 2500 mg, about 200 mg to about 2500 mg, about 300 mg to about 2500 mg, about 400 mg to about 2500 mg, about 500 mg to about 2500 mg, about 600 mg to about 2500 mg, about 700 mg to about 2500 mg, about 800 mg to about 2500 mg, about 800 mg to about 2500 mg, about 900 mg to about 2500 mg, about 1000 mg to about 2500 mg, about 1100 mg to about 2500 mg, about 1200 mg to about 2500 mg, about 100 mg to about 2400 mg, about 100 mg to about 2300 mg, about 100 mg to about 2200 mg, about 100 mg to about 2100 mg, about 100 mg to about 2000 mg, about 100 mg to about 1900 mg, about 100 mg to about 1800 mg, about 100 mg, to about 1700 mg, about 100 mg to about 1600 mg, about 100 mg to about 1500 mg, about 100 mg to about 1400 mg, about 100 mg to about 1300 mg, about 100 mg to about 1200 mg, about 100 mg to about 1100 mg, about 100 mg to about 1000 mg, about 200 mg to about 2500 mg, about 200 mg to about 2400 mg, about 200 mg to about 2300 mg, about 200 mg to about 2200 mg, about 200 mg to about 2100 mg, about 200 mg to about 2000 mg, about 200 mg to about 1900 mg, about 200 mg to about 1800 mg, about 200 mg to about 1700 mg, about 200 mg to about 1600 mg, about 200 mg to about 1500 mg, about 200 mg to about 1400 mg, about 200 mg to about 1300 mg, about 200 mg to about 1200 mg, about 200 mg to about 1100 mg, about 200 mg to about 1000 mg, about 300 mg to about 2500 mg, about 300 mg to about 2400 mg, about 300 mg to about 2300 mg, about 300 mg to about 2200 mg, about 300 mg to about 2100 mg, about 300 mg to about 2000 mg, about 300 mg to about 1900 mg, about 300 mg to about 1800 mg, about 300 mg to about 1700 mg, about 300 mg to about 1600 mg, about 300 mg to about 1500 mg, about 300 mg to about 1400 mg, about 300 mg to about 1300 mg, about 300 mg to about 1200 mg, about 300 mg to about 1100 mg, about 300 mg to about 1000 mg, about 400 mg to about 2500 mg, about 400 mg to about 2400 mg, about 400 mg to about 2300 mg, about 400 mg to about 2200 mg, about 400 mg to about 2100 mg, about 400 mg to about 2000 mg, about 400 mg to about 1900 mg, about 400 mg to about 1800 mg, about 400 mg to about 1700 mg, about 400 mg to about 1600 mg, about 400 mg to about 1500 mg, about 400 mg to about 1400 mg, about 400 mg to about 1300 mg, about 400 mg to about 1200 mg, about 400 mg to about 1100 mg, about 400 mg to about 1000 mg, about 500 mg to about 2500 mg, about 500 mg to about 2400 mg, about 500 mg to about 2300 mg, about 500 mg to about 2200 mg, about 500 mg to about 2100 mg, about 500 mg to about 2000 mg, about 500 mg to about 1900 mg, about 500 mg to about 1800 mg, about 500 mg to about 1700 mg, about 500 mg to about 1600 mg, about 500 mg to about 1500 mg, about 500 mg to about 1400 mg, about 500 mg to about 1300 mg, about 500 mg to about 1200 mg, about 500 mg to about 1100 mg, about 500 mg to about 1000 mg, about 600 mg to about 2500 mg, about 600 mg to about 2400 mg, about 600 mg to about 2300 mg, about 600 mg to about 2200 mg, about 600 mg to about 2100 mg, about 600 mg to about 2000 mg, about 600 mg to about 1900 mg, about 600 mg to about 1800 mg, about 600 mg to about 1700 mg, about 600 mg to about 1600 mg, about 600 mg to about 1500 mg, about 600 mg to about 1400 mg, about 600 mg to about 1300 mg, about 600 mg to about 1200 mg, about 600 mg to about 1100 mg about 300 mg to about 2500 mg, about 700 mg to about 2400 mg, about 700 mg to about 2300 mg, about 700 mg to about 2200 mg, about 700 mg to about 2100 mg, about 700 mg to about 2000 mg, about 700 mg to about 1900 mg, about 700 mg to about 1800 mg, about 700 mg to about 1700 mg, about 700 mg to about 1600 mg, about 700 mg to about 1500 mg, about 700 mg to about 1400 mg, about 700 mg to about 1300 mg, about 700 mg to about 1200 mg, about 300 mg to about 2500 mg, about 300 mg to about 2400 mg, about 800 mg to about 2300 mg, about 800 mg to about 2200 mg, about 800 mg to about 2100 mg, about 800 mg to about 2000 mg, about 800 mg to about 1900 mg, about 800 mg to about 1800 mg, about 800 mg to about 1700 mg, about 800 mg to about 1600 mg, about 800 mg to about 1500 mg, about 800 mg to about 1400 mg, about 800 mg to about 1300 mg, about 900 mg to about 2500 mg, about 900 mg to about 2400 mg, about 900 mg to about 2300 mg, about 900 mg to about 2200 mg, about 900 mg to about 2100 mg, about 900 mg to about 2000 mg, about 900 mg to about 1900 mg, about 900 mg to about 1800 mg, about 900 mg to about 1700 mg, about 900 mg to about 1600 mg, about 900 mg to about 1500 mg, about 900 mg to about 1400 mg, about 1000 mg to about 2500 mg, about 1000 mg to about 2400 mg, about 1000 mg to about 2300 mg, about 1000 mg to about 2200 mg, about 1000 mg to about 2100 mg, about 1000 mg to about 2000 mg, about 1000 mg to about 1900 mg, about 1000 mg to about 1800 mg, about 1000 mg to about 1700 mg, about 1000 mg to about 1600 mg, about 1000 mg to about 1500 mg, about 1100 mg to about 2500 mg, about 1100 mg to about 2400 mg, about 1100 mg to about 2300 mg, about 1100 mg to about 2200 mg, about 1100 mg to about 2100 mg, about 1100 mg to about 2000 mg, about 1100 mg to about 1900 mg, about 1100 mg to about 1800 mg, about 1100 mg to about 1700 mg, about 1100 mg to about 1600 mg, about 1200 mg to about 2500 mg, about 1200 mg to about 2400 mg, about 1200 mg to about 2300 mg, about 1200 mg to about 2200 mg, about 1200 mg to about 2100 mg, about 1200 mg to about 2000 mg, about 1200 mg to about 1900 mg, about 1200 mg to about 1800 mg, about 1200 mg to about 1700 mg, about 1300 mg to about 2500 mg, about 1300 mg to about 2400 mg, about 1300 mg to about 2300 mg, about 1300 mg to about 2200 mg, about 1300 mg to about 2100 mg, about 1300 mg to about 2000 mg, about 1300 mg to about 1900 mg, about 1300 mg to about 1800 mg, about 1400 mg to about 2500 mg, about 1400 mg to about 2400 mg, about 1400 mg to about 2300 mg, about 1400 mg to about 2200 mg, about 1400 mg to about 2100 mg, about 1400 mg to about 2000 mg, about 1400 mg to about 1900 mg, about 1500 mg to about 2500 mg, about 1500 mg to about 2400 mg, about 1500 mg to about 2300 mg, about 1500 mg to about 2200 mg, about 1500 mg to about 2100 mg, about 1500 mg to about 2000 mg, about 1600 mg to about 2500 mg, about 1600 mg to about 2400 mg, about 1600 mg to about 2300 mg, about 1600 mg to about 2200 mg, or about 1600 mg to about 2100 mg.

In some aspects, the one or more glucocorticoid receptor antagonist, or pharmaceutically acceptable salt thereof, is mifepristone that is administered in a total daily amount of about 100 mg, about 110 mg, about 115 mg, about 120 mg, about 125 mg, about 130 mg, about 135 mg, about 140 mg, about 145 mg, about 150 mg, about 160 mg, about 170 mg, about 175 mg, about 180 mg, about 190 mg, about 200 mg, about 225 mg, about 250 mg, about 275 mg, about 300 mg, about 325 mg, about 350 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2100 mg, about 2200 mg, about 2300 mg, about 2400 mg, or about 2500 mg.

In some aspects, the one or more immune checkpoint inhibitors comprises or is a PD-1 antagonist. In some aspects, the one or more immune checkpoint inhibitors comprises or is a PD-L1 antagonist. In some aspects, the one or more immune checkpoint inhibitors comprises or is a CTLA-4 antagonist. In some aspects, the one or more immune checkpoint inhibitors are a PD-1 antagonist and a CTLA-4 antagonist. In some aspects, the one or more immune checkpoint inhibitors are a PD-L1 antagonist and a CTLA-4 antagonist. In some aspects, the one or more checkpoint inhibitors are a PD-1 antagonist, a PD-L1 antagonist, and a CTLA-4 antagonist.

In some aspects, the one or more immune checkpoint inhibitors (e.g., PD-1 antagonist, PD-L1 antagonist, CTLA-4 antagonist) comprise an antibody.

### PD-1 Antagonists

In some aspects, the one or more PD-1 antagonist is selected from the group consisting of pembrolizumab, nivolumab, cemiplimab, sintilimab, dostarlimab, tisleizumab, torpipalimab, spartalizumab, camrelizumab, lambrolizumab, AMP-224, pidilizumab, or any combinations thereof. In some aspects, a therapeutically effective amount of one or more PD-1 antagonists is administered in a therapeutically effective amount.

In some aspects, the PD-1 antagonist is pembrolizumab. In some aspect, a therapeutically effective amount of pembrolizumab is administered to the subject. In some aspects, the pembrolizumab is administered at a single dose of about 100 mg to about 600 mg. In some aspects, the pembrolizumab is administered at a single dose of about 100 mg, about 110 mg, about 120 mg, about 125 mg, about 130 mg, about 140 mg, about 150 mg, about 160 mg, about 170 mg, about 175 mg, about 180 mg, about 190 mg, about 200 mg, about 210 mg, about 220 mg, about 225 mg, about 230 mg, about 240 mg, about 250 mg, about 260 mg, about 270 mg, about 275 mg, about 280 mg, about 290 mg, about 300 mg, about 310 mg, about 320 mg, about 325 mg, about 330 mg, about 340 mg, about 350 mg, about 360 mg, about 370 mg, about 375 mg, about 380 mg, about 390 mg, about 400 mg, about 410 mg, about 420 mg, about 425 mg, about 430 mg, about 440 mg, about 450 mg, about 460 mg, about 470 mg, about 475 mg, about 480 mg, about 490 mg, about 500 mg, about 510 mg, about 520 mg, about 525 mg, about 540 mg, 550 mg, about 560 mg, about 570 mg, about 575 mg, about 580 mg, about 590 mg, or about 600 mg.

In some aspects, the single dose of pembrolizumab is administered once weekly, once every two weeks, once every three weeks, once every four weeks, once every five weeks, or once every six weeks. In some aspects, the single dose of pembrolizumab is administered once every three weeks. In some aspects, the single dose of pembrolizumab is administered once every six weeks.

In some aspects, the single dose of pembrolizumab is about 200 mg administered once every three weeks. In some aspects, the single dose of pembrolizumab is about 400 mg administered once every six weeks.

In some aspects, the PD-1 antagonist is nivolumab. In some aspects, a therapeutically effective amount of nivolumab is administered to the subject. In some aspects, the nivolumab is administered at a single dose of about 0.5 mg/kg to about 7.5 mg/kg. In some aspects, the nivolumab is administered at a single dose of about 0.5 mg/kg, about 0.75 mg/kg, about 1 mg/kg, about 1.25 mg/kg, about 1.5 mg/kg, about 1.75 mg/kg, about 2 mg/kg, about 2.25 mg/kg, about 2.5 mg/kg, about 2.75 mg/kg, about 3 mg/kg, about 3.25 mg/kg, about 3.5 mg/kg, about 3.75 mg/kg, about 4 mg/kg, about 4.25 mg/kg, about 4.5 mg/kg, about 4.75 mg/kg, about 5 mg/kg, about 5.25 mg/kg, about 5.5 mg/kg, about 5.75 mg/kg, about 6 mg/kg, about 6.25 mg/kg, about 6.5 mg/kg, about 6.75 mg/kg, about 7 mg/kg, about 7.25 mg/kg, or about 7.5 mg/kg.

In some aspects, the nivolumab is administered at a single dose of about 100 mg to about 750 mg. In some aspects, the nivolumab is administered at a single dose of about 100 mg, about 110 mg, about 120 mg, about 125 mg, about 130 mg, about 140 mg, about 150 mg, about 160 mg, about 170 mg, about 175 mg, about 180 mg, about 190 mg, about 200 mg, about 210 mg, about 220 mg, about 225 mg, about 230 mg, about 240 mg, about 250 mg, about 260 mg, about 270 mg, about 275 mg, about 280 mg, about 290 mg, about 300 mg, about 310 mg, about 320 mg, about 325 mg, about 330 mg, about 340 mg, about 350 mg, about 360 mg, about 370 mg, about 375 mg, about 380 mg, about 390 mg, about 400 mg, about 410 mg, about 420 mg, about 425 mg, about 430 mg, about 440 mg, about 450 mg, about 460 mg, about 470 mg, about 475 mg, about 480 mg, about 490 mg, about 500 mg, about 510 mg, about 520 mg, about 525 mg, about 530 mg, about 540 mg, about 550 mg, about 560 mg, about 570 mg, about 575 mg, about 580 mg, about 590 mg, about 600 mg, about 610 mg, about 620 mg, about 625 mg, about 630 mg, about 640 mg, about 650 mg, about 660 mg, about 670 mg, about 675 mg, about 680 mg, about 690 mg, about 700 mg, about 710 mg, about 720 mg, about 725 mg, about 730 mg, about 740 mg, or about 750 mg.

In some aspects, the single dose of nivolumab is administered once weekly, once every two weeks, once every three weeks, once every four weeks, once every five weeks, or once every six weeks. In some aspects, the single dose of nivolumab is administered once every two weeks. In some aspects, the single dose of nivolumab is administered once every three weeks.

In some aspects, the single dose of nivolumab is about 1 mg/kg administered once every three weeks. In some aspects, the single dose of nivolumab is about 3 mg/kg administered once every two weeks. In some aspects, the single dose of nivolumab is about 3 mg/kg administered once every three weeks. In some aspects, the single dose of nivolumab is about 240 mg administered once every two weeks. In some aspects, the single dose of nivolumab is about 360 mg administered once every three weeks.

In some aspects, the PD-1 antagonist is cemiplimab. In some aspects, a therapeutically effective amount of cemiplimab is administered to the subject. In some aspects, the cemiplimab is administered at a single dose of about 100 mg to about 500 mg. In some aspects, the cemiplimab is administered at a single dose of about 100 mg, about 110 mg, about 120 mg, about 125 mg, about 130 mg, about 140 mg, about 150 mg, about 160 mg, about 170 mg, about 175 mg, about 180 mg, about 190 mg, about 200 mg, about 210 mg, about 220 mg, about 225 mg, about 230 mg, about 240 mg, about 250 mg, about 260 mg, about 270 mg, about 275 mg, about 280 mg, about 290 mg, about 300 mg, about 310 mg, about 320 mg, about 325 mg, about 330 mg, about 340 mg, about 350 mg, about 360 mg, about 370 mg, about 375 mg, about 380 mg, about 390 mg, about 400 mg, about 410 mg, about 420 mg, about 425 mg, about 430 mg, about 440 mg, about 450 mg, about 460 mg, about 470 mg, about 475 mg, about 480 mg, about 490 mg, or about 500 mg.

In some aspects, the single dose of cemiplimab is administered once weekly, once every two weeks, once every three weeks, once every four weeks, once every five weeks, or once every six weeks. In some aspects, the single dose of cemiplimab is administered once every three weeks.

In some aspects, the single dose of cemiplimab is about 350 mg administered once every three weeks.

In some aspects, the PD-1 antagonist is sintilimab. In some aspects, a therapeutically effective amount of sintilimab is administered to the subject. In some aspects, the sintilimab is administered at a single dose of about 25 mg to about 500 mg. In some aspects, the sintilimab is administered at a single dose of about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 125 mg, about 130 mg, about 140 mg, about 150 mg, about 160 mg, about 170 mg, about 175 mg, about 180 mg, about 190 mg, about 200 mg, about 210 mg, about 220 mg, about 225 mg, about 230 mg, about 240 mg, about 250 mg, about 260 mg, about 270 mg, about 275 mg, about 280 mg, about 290 mg, about 300 mg, about 310 mg, about 320 mg, about 325 mg, about 330 mg, about 340 mg, about 350 mg, about 360 mg, about 370 mg, about 375 mg, about 380 mg, about 390 mg, about 400 mg, about 410 mg, about 420 mg, about 425 mg, about 430 mg, about 440 mg, about 450 mg, about 460 mg, about 470 mg, about 475 mg, about 480 mg, about 490 mg, or about 500 mg.

In some aspects, the single dose of sintilimab is administered once weekly, once every two weeks, once every three weeks, once every four weeks, once every five weeks, or once every six weeks. In some aspects, the single dose of sintilimab is administered once every three weeks.

In some aspects, the single dose of sintilimab is about 100 mg administered once every three weeks. In some aspects, the single dose of sintilimab is about 200 mg administered once every three weeks.

In some aspects, the PD-1 antagonist is dostarlimab. In some aspects, a therapeutically effective amount of dostarlimab is administered to the subject. In some aspects, the dostarlimab is administered at a single dose of about 100 mg to about 1500 mg. In some aspects, the nivolumab is administered at a single dose of about 100 mg, about 110 mg, about 120 mg, about 125 mg, about 130 mg, about 140 mg, about 150 mg, about 160 mg, about 170 mg, about 175 mg, about 180 mg, about 190 mg, about 200 mg, about 210 mg, about 220 mg, about 225 mg, about 230 mg, about 240 mg, about 250 mg, about 260 mg, about 270 mg, about 275 mg, about 280 mg, about 290 mg, about 300 mg, about 310 mg, about 320 mg, about 325 mg, about 330 mg, about 340 mg, about 350 mg, about 360 mg, about 370 mg, about 375 mg, about 380 mg, about 390 mg, about 400 mg, about 410 mg, about 420 mg, about 425 mg, about 430 mg, about 440 mg, about 450 mg, about 460 mg, about 470 mg, about 475 mg, about 480 mg, about 490 mg, about 500 mg, about 510 mg, about 520 mg, about 525 mg, about 530 mg, about 540 mg, about 550 mg, about 560 mg, about 570 mg, about 575 mg, about 580 mg, about 590 mg, about 600 mg, about 610 mg, about 620 mg, about 625 mg, about 630 mg, about 640 mg, about 650 mg, about 660 mg, about 670 mg, about 675 mg, about 680 mg, about 690 mg, about 700 mg, about 710 mg, about 720 mg, about 725 mg, about 730 mg, about 740 mg, about 750 mg, about 760 mg, about 770 mg, about 775 mg, about 780 mg, about 790 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, or about 1500 mg.

In some aspects, the single dose of dostarlimab is administered once weekly, once every two weeks, once every three weeks, once every four weeks, once every five weeks, or once every six weeks. In some aspects, the single dose of dostarlimab is administered once every three weeks. In some aspects, the single dose of dostarlimab is administered once every six weeks.

In some aspects, the single dose of dostarlimab is about 500 mg administered once every three weeks. In some aspects, the single dose of dostarlimab is about 1000 mg administered once every six weeks. In some aspects, the single dose of dostarlimab is about 500 mg administered once every three weeks for the first four doses and then about 1000 mg every six weeks.

In some aspects, the PD-1 antagonist is tislelizumab. In some aspects, a therapeutically effective amount of tislelizumab is administered to the subject. In some aspects, the tislelizumab is administered at a single dose of about 50 mg to about 500 mg. In some aspects, the tislelizumab is administered at a single dose of about 50 mg, about 60 mg, about 70 mg, about 75 mg, about 80 mg, about 90 mg, about 100 mg, about 110 mg, about 120 mg, about 125 mg, about 130 mg, about 140 mg, about 150 mg, about 160 mg, about 170 mg, about 175 mg, about 180 mg, about 190 mg, about 200 mg, about 210 mg, about 220 mg, about 225 mg, about 230 mg, about 240 mg, about 250 mg, about 260 mg, about 270 mg, about 275 mg, about 280 mg, about 290 mg, about 300 mg, about 310 mg, about 320 mg, about 325 mg, about 330 mg, about 340 mg, about 350 mg, about 360 mg, about 370 mg, about 375 mg, about 380 mg, about 390 mg, about 400 mg, about 410 mg, about 420 mg, about 425 mg, about 430 mg, about 440 mg, about 450 mg, about 460 mg, about 470 mg, about 475 mg, about 480 mg, about 490 mg, or about 500 mg.

In some aspects, the single dose of tislelizumab is administered once weekly, once every two weeks, once every three weeks, once every four weeks, once every five weeks, or once every six weeks. In some aspects, the single dose of tislelizumab is administered once every three weeks.

In some aspects, the single dose of tislelizumab is about 200 mg administered once every three weeks.

In some aspects, the PD-1 antagonist is toripalimab. In some aspects, a therapeutically effective amount of toripalimab is administered to the subject. In some aspects, the toripalimab is administered at a single dose of about 1 mg/kg to about 7 mg/kg. In some aspects, the toripalimab is administered at a single dose of about 1 mg/kg, about 1.25 mg/kg, about 1.5 mg/kg, about 1.75 mg/kg, about 2 mg/kg, about 2.25 mg/kg, about 2.5 mg/kg, about 2.75 mg/kg, about 3 mg/kg, about 3.25 mg/kg, about 3.5 mg/kg, about 3.75 mg/kg, about 4 mg/kg, about 4.25 mg/kg, about 4.5 mg/kg, about 4.75 mg/kg, about 5 mg/kg, about 5.25 mg/kg, about 5.5 mg/kg, about 5.75 mg/kg, about 6 mg/kg, about 6.25 mg/kg, about 6.5 mg/kg, about 6.75 mg/kg, or about 7 mg/kg.

In some aspects, the single dose of toripalimab is administered once weekly, once every two weeks, once every three weeks, once every four weeks, once every five weeks, or once every six weeks. In some aspects, the single dose of toripalimab is administered once every two weeks.

In some aspects, the single dose of toripalimab is about 3 mg/kg administered once every two weeks.

In some aspects, the PD-1 antagonist is spartalizumab. In some aspects, a therapeutically effective amount of spartalizumab is administered to the subject. In some aspects, the spartalizumab is administered at a single dose of about 100 mg to about 700 mg. In some aspects, the nivolumab is administered at a single dose of about 100 mg, about 110 mg, about 120 mg, about 125 mg, about 130 mg, about 140 mg, about 150 mg, about 160 mg, about 170 mg, about 175 mg, about 180 mg, about 190 mg, about 200 mg, about 210 mg, about 220 mg, about 225 mg, about 230 mg, about 240 mg, about 250 mg, about 260 mg, about 270 mg, about 275 mg, about 280 mg, about 290 mg, about 300 mg, about 310 mg, about 320 mg, about 325 mg, about 330 mg, about 340 mg, about 350 mg, about 360 mg, about 370 mg, about 375 mg, about 380 mg, about 390 mg, about 400 mg, about 410 mg, about 420 mg, about 425 mg, about 430 mg, about 440 mg, about 450 mg, about 460 mg, about 470 mg, about 475 mg, about 480 mg, about 490 mg, about 500 mg, about 510 mg, about 520 mg, about 525 mg, about 530 mg, about 540 mg, about 550 mg, about 560 mg, about 570 mg, about 575 mg, about 580 mg, about 590 mg, about 600 mg, about 610 mg, about 620 mg, about 625 mg, about 630 mg, about 640 mg, about 650 mg, about 660 mg, about 670 mg, about 675 mg, about 680 mg, about 690 mg, or about 700 mg.

In some aspects, the single dose of spartalizumab is administered once weekly, once every two weeks, once every three weeks, once every four weeks, once every five weeks, or once every six weeks. In some aspects, the single dose of spartalizumab is administered once every three weeks. In some aspects, the spartalizumab is administered once every four weeks.

In some aspects, the spartalizumab is administered at single dose of about 300 mg every three weeks. In some aspects, the spartalizumab is administered at a single dose of about 400 mg every four weeks.

In some aspects, the PD-1 antagonist is camrelizumab. In some aspects, a therapeutically effective amount of camrelizumab is administered to the subject. In some aspects, the camrelizumab is administered at a single dose of about 50 mg to about 500 mg. In some aspects, the camrelizumab is administered at a single dose of about 50 mg, about 60 mg, about 70 mg, about 75 mg, about 80 mg, about 90 mg, about 100 mg, about 110 mg, about 120 mg, about 125 mg, about 130 mg, about 140 mg, about 150 mg, about 160 mg, about 170 mg, about 175 mg, about 180 mg, about 190 mg, about 200 mg, about 210 mg, about 220 mg, about 225 mg, about 230 mg, about 240 mg, about 250 mg, about 260 mg, about 270 mg, about 275 mg, about 280 mg, about 290 mg, about 300 mg, about 310 mg, about 320 mg, about 325 mg, about 330 mg, about 340 mg, about 350 mg, about 360 mg, about 370 mg, about 375 mg, about 380 mg, about 390 mg, about 400 mg, about 410 mg, about 420 mg, about 425 mg, about 430 mg, about 440 mg, about 450 mg, about 460 mg, about 470 mg, about 475 mg, about 480 mg, about 490 mg, or about 500 mg.

In some aspects, the single dose of camrelizumab is administered once weekly, once every two weeks, once every three weeks, once every four weeks, once every five weeks, or once every six weeks. In some aspects, the single dose of camrelizumab is administered once every three weeks.

In some aspects, the single dose of camrelizumab is about 200 mg administered once every three weeks.

In some aspects, the PD-1 antagonist is lambrolizumab. In some aspects, a therapeutically effective amount of lambrolizumab is administered to the subject. In some aspects, the lambrolizumab is administered at a single dose of about 0.5 mg/kg to about 15 mg. In some aspects, the lambrolizumab is administered at a single dose of about 0.5 mg/kg, about 0.75 mg/kg, about 1 mg/kg, about 1.25 mg/kg, about 1.5 mg/kg, about 1.75 mg/kg, about 2 mg/kg, about 2.25 mg/kg, about 2.5 mg/kg, about 2.75 mg/kg, about 3 mg/kg, about 3.25 mg/kg, about 3.5 mg/kg, about 3.75 mg/kg, about 4 mg/kg, about 4.25 mg/kg, about 4.5 mg/kg, about 4.75 mg/kg, about 5 mg/kg, about 5.25 mg/kg, about 5.5 mg/kg, about 5.75 mg/kg, about 6 mg/kg, about 6.25 mg/kg, about 6.5 mg/kg, about 6.75 mg/kg, about 7 mg/kg, about 7.25 mg/kg, about 7.5 mg/kg, about 7.75 mg/kg, about 8 mg/kg, about 8.25 mg/kg, about 8.5 mg/kg, about 8.75 mg/kg, about 9 mg/kg, about 9.25 mg/kg, about 9.5 mg/kg, about 9.75 mg/kg, about 10 mg/kg, about 10.25 mg/kg, about 10.5 mg/kg, about 10.75 mg/kg, about 11 mg/kg, about 11.25 mg/kg, about 11.5 mg/kg, about 11.75 mg/kg, about 12 mg/kg, about 12.25 mg/kg, about 12.5 mg/kg, about 12.75 mg/kg, about 13 mg/kg, about 13.25 mg/kg, about 13.5 mg/kg, about 13.75 mg/kg, about 14 mg/kg, about 14.25 mg/kg, about 14.25 mg/kg, about 14.5 mg/kg, about 14.75 mg/kg, or about 15 mg/kg.

In some aspects, the single dose of lambrolizumab is administered once weekly, once every two weeks, once every three weeks, once every four weeks, once every five weeks, or once every six weeks.

In some aspects, the PD-1 antagonist is AMP-224. In some aspects, a therapeutically effective amount of AMP-224 is administered to the subject. In some aspects, the AMP-224 is administered at a single dose of about 1 mg/kg to about 15 mg/kg. In some aspects, the AMP-224 is administered at a single dose of about 1 mg/kg, about 1.25 mg/kg, about 1.5 mg/kg, about 1.75 mg/kg, about 2 mg/kg, about 2.25 mg/kg, about 2.5 mg/kg, about 2.75 mg/kg, about 3 mg/kg, about 3.25 mg/kg, about 3.5 mg/kg, about 3.75 mg/kg, about 4 mg/kg, about 4.25 mg/kg, about 4.5 mg/kg, about 4.75 mg/kg, about 5 mg/kg, about 5.25 mg/kg, about 5.5 mg/kg, about 5.75 mg/kg, about 6 mg/kg, about 6.25 mg/kg, about 6.5 mg/kg, about 6.75 mg/kg, about 7 mg/kg, about 7.25 mg/kg, about 7.5 mg/kg, about 7.75 mg/kg, about 8 mg/kg, about 8.25 mg/kg, about 8.5 mg/kg, about 8.75 mg/kg, about 9 mg/kg, about 9.25 mg/kg, about 9.5 mg/kg, about 9.75 mg/kg, about 10 mg/kg, about 10.25 mg/kg, about 10.5 mg/kg, about 10.75 mg/kg, about 11 mg/kg, about 11.25 mg/kg, about 11.5 mg/kg, about 11.75 mg/kg, about 12 mg/kg, about 12.25 mg/kg, about 12.5 mg/kg, about 12.75 mg/kg, about 13 mg/kg, about 13.25 mg/kg, about 13.5 mg/kg, about 13.75 mg/kg, about 14 mg/kg, about 14.25 mg/kg, about 14.25 mg/kg, about 14.5 mg/kg, about 14.75 mg/kg, or about 15 mg/kg.

In some aspects, the single dose of AMP-224 is administered once weekly, once every two weeks, once every three weeks, once every four weeks, once every five weeks, or once every six weeks.

In some aspects, the PD-1 antagonist is pidilizumab. In some aspects, a therapeutically effective amount of pidilizumab is administered to the subject. In some aspects, the pidilizumab is administered at a single dose of about 0.5 mg/kg to about 5 mg/kg. In some aspects, the pidilizumab is administered at a single dose of about 0.5 mg/kg, about 0.75 mg/kg, about 1 mg/kg, about 1.25 mg/kg, about 1.5 mg/kg, about 1.75 mg/kg, about 2 mg/kg, about 2.25 mg/kg, about 2.5 mg/kg, about 2.75 mg/kg, about 3 mg/kg, about 3.25 mg/kg, about 3.5 mg/kg, about 3.75 mg/kg, about 4 mg/kg, about 4.25 mg/kg, about 4.5 mg/kg, about 4.75 mg/kg, or about 5 mg/kg.

In some aspects, the single dose of pidilizumab is administered once weekly, once every two weeks, once every three weeks, once every four weeks, once every five weeks, or once every six weeks.

### PD-L1 Antagonists

In some aspects, the one or more PD-L1 antagonist is selected from the group consisting of atezolizumab, durvalumab, avelumab, KN035, MDX-1105, MEDI4736, MPDL3280A, BMS-936559, and combinations thereof. In some aspects, a therapeutically effective amount of the one or more PD-L1 antagonist is administered to the subject.

Whereas the MHC-I antigen presentation pathway is crucial for the recognition of tumor cells by CD8+ T cells, PD-L1 is one of the key immune inhibitory ligands expressed by cancer cells, as shown in Zamora, A. E., Crawford, J. C. & Thomas, P. G. Hitting the Target: How T Cells Detect and Eliminate Tumors. J Immunol 200, 392-399 (2018) and Dong, H., et al. Tumor-associated B7-H1 promotes T-cell apoptosis: a potential mechanism of immune evasion. Nat Med 8, 793-800 (2002). Upon binding to its cognate receptor PD-1 on tumor-infiltrating cytotoxic T lymphocytes (CTLs), PD-L1 induces an inhibitory signal to dampen their tumor-killing activity, reviewed in Pardoll, D. M. The blockade of immune checkpoints in cancer immunotherapy. Nat Rev Cancer 12, 252-264 (2012).

In some aspects, the PD-L1 antagonist is atezolizumab. In some aspects, a therapeutically effective amount of atezolizumab is administered to the subject. In some aspects, atezolizumab is administered at single dose of about 500 mg to about 2500 mg. In some aspects, the atezolizumab is administered at a single dose of about 500 mg, about 510 mg, about 520 mg, about 525 mg, about 530 mg, about 540 mg, about 550 mg, about 560 mg, about 570 mg, about 575 mg, about 580 mg, about 590 mg, about 600 mg, about 610 mg, about 620 mg, about 625 mg, about 630 mg, about 640 mg, about 650 mg, about 660 mg, about 670 mg, about 675 mg, about 680 mg, about 690 mg, about 700 mg, about 710 mg, about 720 mg, about 725 mg, about 730 mg, about 740 mg, about 750 mg, about 760 mg, about 770 mg, about 775 mg, about 780 mg, about 790 mg, about 800 mg, about 810 mg, about 820 mg, about 825 mg, about 830 mg, about 840 mg, about 850 mg, about 860 mg, about 870 mg, about 875 mg, about 880 mg, about 890 mg, about 900 mg, about 910 mg, about 920 mg, about 925 mg, about 930 mg, about 940 mg, about 950 mg, about 960 mg, about 970 mg, about 975 mg, about 980 mg, about 990 mg, about 1000 mg, about 1010 mg, about 1020 mg, about 1025 mg, about 1030 mg, about 1040 mg, about 1050 mg, about 1060 mg, about 1070 mg, about 1075 mg, about 1080 mg, about 1090 mg, about 1100 mg, about 1110 mg, about 1120 mg, about 1125 mg, about 1130 mg, about 1140 mg, about 1150 mg, about 1160 mg, about 1170 mg, about 1175 mg, about 1180 mg, about 1190 mg, about 1200 mg, about 1210 mg, about 1220 mg, about 1225 mg, about 1230 mg, about 1240 mg, about 1250 mg, about 1260 mg, about 1270 mg, about 1275 mg, about 1280 mg, about 1290 mg, about 1300 mg, about 1310 mg, about 1320 mg, about 1325 mg, about 1330 mg, about 1340 mg, about 1350 mg, about 1360 mg, about 1370 mg, about 1375 mg, about 1380 mg, about 1390 mg, about 1400 mg, about 1410 mg, about 1420 mg, about 1425 mg, about 1430 mg, about 1440 mg, about 1450 mg, about 1460 mg, about 1470 mg, about 1475 mg, about 1480 mg, about 1490 mg, about 1500 mg, about 1510 mg, about 1520 mg, about 1525 mg, about 1530 mg, about 1540 mg, about 1550 mg, about 1560 mg, about 1570 mg, about 1575 mg, about 1580 mg, about 1590 mg, about 1600 mg, about 1610 mg, about 1620 mg, about 1625 mg, about 1630 mg, about 1640 mg, about 1650 mg, about 1660 mg, about 1670 mg, about 1675 mg, about 1680 mg, about 1690 mg, about 1700 mg, about 1710 mg, about 1720 mg, about 1725 mg, about 1730 mg, about 1740 mg, about 1750 mg, about 1760 mg, about 1770 mg, about 1775 mg, about 1780 mg, about 1790 mg, about 1800 mg, about 1810 mg, about 1820 mg, about 1825 mg, about 1830 mg, about 1840 mg, about 1850 mg, about 1860 mg, about 1870 mg, about 1875 mg, about 1880 mg, about 1890 mg, about 1900 mg, about 1910 mg, about 1920 mg, about 1925 mg, about 1930 mg, about 1940 mg, about 1950 mg, about 1960 mg, about 1970 mg, about 1975 mg, about 1980 mg, about 1990 mg, about 2000 mg, about 2010 mg, about 2020 mg, about 2030 mg, about 2040 mg, about 2050 mg, about 2060 mg, about 2070 mg, about 2080 mg, about 2090 mg, about 2100 mg, about 2110 mg, about 2120 mg, about 2130 mg, about 2140 mg, about 2150 mg, about 2160 mg, about 2170 mg, about 2180 mg, about 2190 mg, about 2200 mg, about 2210 mg, about 2220 mg, about 2230 mg, about 2240 mg, about 2250 mg, about 2260 mg, about 2270 mg, about 2280 mg, about 2290 mg, about 2300 mg, about 2310 mg, about 2320 mg, about 2330 mg, about 2340 mg, about 2350 mg, about 2360 mg, about 2370 mg, about 2380 mg, about 2390 mg, about 2400 mg, about 2410 mg, about 2420 mg, about 2430 mg, about 2440 mg, about 2450 mg, about 2460 mg, about 2470 mg, about 2480 mg, about 2490 mg, or about 2500 mg.

In some aspects, the single dose of atezolizumab is administered once weekly, once every two weeks, once every three weeks, once every four weeks, once every five weeks, or once every six weeks. In some aspects, the single dose of atezolizumab is administered once every two weeks. In some aspects, the single dose of atezolizumab is administered once every three weeks. In some aspects, the single dose of atezolizumab is administered once every four weeks.

In some aspects, the single dose of atezolizumab is about 840 mg administered once every two weeks. In some aspects, the single dose of atezolizumab is about 1200 mg administered once every three weeks. In some aspects, the single dose of atezolizumab is about 1680 mg administered once every four weeks.

In some aspects, the PD-L1 antagonist is durvalumab. In some aspects, a therapeutically effective amount of durvalumab is administered to the subject. In some aspects, the durvalumab is administered at a single dose of about 750 mg to about 2500 mg. In some aspects, the durvalumab is administered at a single dose of about 750 mg, about 760 mg, about 770 mg, about 775 mg, about 780 mg, about 790 mg, about 800 mg, about 810 mg, about 820 mg, about 825 mg, about 830 mg, about 840 mg, about 850 mg, about 860 mg, about 870 mg, about 875 mg, about 880 mg, about 890 mg, about 900 mg, about 910 mg, about 920 mg, about 925 mg, about 930 mg, about 940 mg, about 950 mg, about 960 mg, about 970 mg, about 975 mg, about 980 mg, about 990 mg, about 1000 mg, about 1010 mg, about 1020 mg, about 1025 mg, about 1030 mg, about 1040 mg, about 1050 mg, about 1060 mg, about 1070 mg, about 1075 mg, about 1080 mg, about 1090 mg, about 1100 mg, about 1110 mg, about 1120 mg, about 1125 mg, about 1130 mg, about 1140 mg, about 1150 mg, about 1160 mg, about 1170 mg, about 1175 mg, about 1180 mg, about 1190 mg, about 1200 mg, about 1210 mg, about 1220 mg, about 1225 mg, about 1230 mg, about 1240 mg, about 1250 mg, about 1260 mg, about 1270 mg, about 1275 mg, about 1280 mg, about 1290 mg, about 1300 mg, about 1310 mg, about 1320 mg, about 1325 mg, about 1330 mg, about 1340 mg, about 1350 mg, about 1360 mg, about 1370 mg, about 1375 mg, about 1380 mg, about 1390 mg, about 1400 mg, about 1410 mg, about 1420 mg, about 1425 mg, about 1430 mg, about 1440 mg, about 1450 mg, about 1460 mg, about 1470 mg, about 1475 mg, about 1480 mg, about 1490 mg, about 1500 mg, about 1510 mg, about 1520 mg, about 1525 mg, about 1530 mg, about 1540 mg, about 1550 mg, about 1560 mg, about 1570 mg, about 1575 mg, about 1580 mg, about 1590 mg, about 1600 mg, about 1610 mg, about 1620 mg, about 1625 mg, about 1630 mg, about 1640 mg, about 1650 mg, about 1660 mg, about 1670 mg, about 1675 mg, about 1680 mg, about 1690 mg, about 1700 mg, about 1710 mg, about 1720 mg, about 1725 mg, about 1730 mg, about 1740 mg, about 1750 mg, about 1760 mg, about 1770 mg, about 1775 mg, about 1780 mg, about 1790 mg, about 1800 mg, about 1810 mg, about 1820 mg, about 1825 mg, about 1830 mg, about 1840 mg, about 1850 mg, about 1860 mg, about 1870 mg, about 1875 mg, about 1880 mg, about 1890 mg, about 1900 mg, about 1910 mg, about 1920 mg, about 1925 mg, about 1930 mg, about 1940 mg, about 1950 mg, about 1960 mg, about 1970 mg, about 1975 mg, about 1980 mg, about 1990 mg, about 2000 mg, about 2010 mg, about 2020 mg, about 2030 mg, about 2040 mg, about 2050 mg, about 2060 mg, about 2070 mg, about 2080 mg, about 2090 mg, about 2100 mg, about 2110 mg, about 2120 mg, about 2130 mg, about 2140 mg, about 2150 mg, about 2160 mg, about 2170 mg, about 2180 mg, about 2190 mg, about 2200 mg, about 2210 mg, about 2220 mg, about 2230 mg, about 2240 mg, about 2250 mg, about 2260 mg, about 2270 mg, about 2280 mg, about 2290 mg, about 2300 mg, about 2310 mg, about 2320 mg, about 2330 mg, about 2340 mg, about 2350 mg, about 2360 mg, about 2370 mg, about 2380 mg, about 2390 mg, about 2400 mg, about 2410 mg, about 2420 mg, about 2430 mg, about 2440 mg, about 2450 mg, about 2460 mg, about 2470 mg, about 2480 mg, about 2490 mg, or about 2500 mg.

In some aspects, the single dose of durvalumab is administered once weekly, once every two weeks, once every three weeks, once every four weeks, once every five weeks, or once every six weeks. In some aspects, the single dose of durvalumab is administered once every three weeks. In some aspects, the single dose of durvalumab is administered once every four weeks.

In some aspects, the single dose of durvalumab is about 1500 mg administered once every three weeks. In some aspects, the single dose of durvalumab is about 1500 mg administered once every four weeks.

In some aspects, the PD-L1 antagonist is avelumab. In some aspects, a therapeutically effective amount of avelumab is administered to the subject. In some aspects, the avelumab is administered at a single dose of about 100 mg to about 1500 mg. In some aspects, the avelumab is administered at a single dose of about 100 mg, about 110 mg, about 120 mg, about 125 mg, about 130 mg, about 140 mg, about 150 mg, about 160 mg, about 170 mg, about 175 mg, about 180 mg, about 190 mg, about 200 mg, about 210 mg, about 220 mg, about 225 mg, about 230 mg, about 240 mg, about 250 mg, about 260 mg, about 270 mg, about 275 mg, about 280 mg, about 290 mg, about 300 mg, about 310 mg, about 320 mg, about 325 mg, about 330 mg, about 340 mg, about 350 mg, about 360 mg, about 370 mg, about 375 mg, about 380 mg, about 390 mg, about 400 mg, about 410 mg, about 420 mg, about 425 mg, about 430 mg, about 440 mg, about 450 mg, about 460 mg, about 470 mg, about 475 mg, about 480 mg, about 490 mg, about 500 mg, about 510 mg, about 520 mg, about 525 mg, about 530 mg, about 540 mg, about 550 mg, about 560 mg, about 570 mg, about 575 mg, about 580 mg, about 590 mg, about 600 mg, about 610 mg, about 620 mg, about 625 mg, about 630 mg, about 640 mg, about 650 mg, about 660 mg, about 670 mg, about 675 mg, about 680 mg, about 690 mg, about 700 mg, about 710 mg, about 720 mg, about 725 mg, about 730 mg, about 740 mg, about 750 mg, about 760 mg, about 770 mg, about 775 mg, about 780 mg, about 790 mg, about 800 mg, about 810 mg, about 820 mg, about 825 mg, about 830 mg, about 840 mg, about 850 mg, about 860 mg, about 870 mg, about 875 mg, about 880 mg, about 890 mg, about 900 mg, about 910 mg, about 920 mg, about 925 mg, about 930 mg, about 940 mg, about 950 mg, about 960 mg, about 970 mg, about 975 mg, about 980 mg, about 990 mg, about 1000 mg, about 1010 mg, about 1020 mg, about 1025 mg, about 1030 mg, about 1040 mg, about 1050 mg, about 1060 mg, about 1070 mg, about 1075 mg, about 1080 mg, about 1090 mg, about 1100 mg, about 1110 mg, about 1120 mg, about 1125 mg, about 1130 mg, about 1140 mg, about 1150 mg, about 1160 mg, about 1170 mg, about 1175 mg, about 1180 mg, about 1190 mg, about 1200 mg, about 1210 mg, about 1220 mg, about 1225 mg, about 1230 mg, about 1240 mg, about 1250 mg, about 1260 mg, about 1270 mg, about 1275 mg, about 1280 mg, about 1290 mg, about 1300 mg, about 1310 mg, about 1320 mg, about 1325 mg, about 1330 mg, about 1340 mg, about 1350 mg, about 1360 mg, about 1370 mg, about 1375 mg, about 1380 mg, about 1390 mg, about 1400 mg, about 1410 mg, about 1420 mg, about 1425 mg, about 1430 mg, about 1440 mg, about 1450 mg, about 1460 mg, about 1470 mg, about 1475 mg, about 1480 mg, about 1490 mg, or about 1500 mg.

In some aspects, the single dose of avelumab is administered once weekly, once every two weeks, once every three weeks, once every four weeks, once every five weeks, or once every six weeks. In some aspects, the single dose of avelumab is administered once every two weeks.

In some aspects, the single dose of avelumab is about 800 mg administered once every two weeks.

In some aspects, the PD-L1 antagonist is KN035. In some aspects, a therapeutically effective amount of KN035 is administered to the subject. In some aspects, the KN035 is administered at a single dose of about 50 mg to about 750 mg. In some aspects, the KN035 is administered at a single dose of about 50 mg, about 60 mg, about 70 mg, about 75 mg, about 80 mg, about 90 mg, about 100 mg, about 110 mg, about 120 mg, about 125 mg, about 130 mg, about 140 mg, about 150 mg, about 160 mg, about 170 mg, about 175 mg, about 180 mg, about 190 mg, about 200 mg, about 210 mg, about 220 mg, about 225 mg, about 230 mg, about 240 mg, about 250 mg, about 260 mg, about 270 mg, about 275 mg, about 280 mg, about 290 mg, about 300 mg, about 310 mg, about 320 mg, about 325 mg, about 330 mg, about 340 mg, about 350 mg, about 360 mg, about 370 mg, about 375 mg, about 380 mg, about 390 mg, about 400 mg, about 410 mg, about 420 mg, about 425 mg, about 430 mg, about 440 mg, about 450 mg, about 460 mg, about 470 mg, about 475 mg, about 480 mg, about 490 mg, about 500 mg, about 510 mg, about 520 mg, about 525 mg, about 530 mg, about 540 mg, about 550 mg, about 560 mg, about 570 mg, about 575 mg, about 580 mg, about 590 mg, about 600 mg, about 610 mg, about 620 mg, about 625 mg, about 630 mg, about 640 mg, about 650 mg, about 660 mg, about 670 mg, about 675 mg, about 680 mg, about 690 mg, about 700 mg, about 710 mg, about 720 mg, about 725 mg, about 730 mg, about 740 mg, or about 750 mg.

In some aspects, the single dose of KN035 is administered once weekly, once every two weeks, once every three weeks, once every four weeks, once every five weeks, or once every six weeks. In some aspects, the single dose of KN035 is administered once every week.

In some aspects, the single dose of KN035 is about 300 mg administered once every week.

In some aspects, the PD-L1 antagonist is MEDI4736. In some aspects, a therapeutically effective amount of MEDI4736 is administered to the subject. In some aspects, the MEDI4736 is administered at a single dose of about 0.1 mg/kg to about 20 mg/kg. In some aspects, the MEDI4736 is administered at a single dose of about 0.1 mg/kg, about 0.25 mg/kg, about 0.5 mg/kg, about 0.75 mg/kg, about 1 mg/kg, about 1.25 mg/kg, about 1.5 mg/kg, about 1.75 mg/kg, about 2 mg/kg, about 2.25 mg/kg, about 2.5 mg/kg, about 2.75 mg/kg, about 3 mg/kg, about 3.25 mg/kg, about 3.5 mg/kg, about 3.75 mg/kg, about 4 mg/kg, about 4.25 mg/kg, about 4.5 mg/kg, about 4.75 mg/kg, about 5 mg/kg, about 5.25 mg/kg, about 5.5 mg/kg, about 5.75 mg/kg, about 6 mg/kg, about 6.25 mg/kg, about 6.5 mg/kg, about 6.75 mg/kg, about 7 mg/kg, about 7.25 mg/kg, about 7.5 mg/kg, about 7.75 mg/kg, about 8 mg/kg, about 8.25 mg/kg, about 8.5 mg/kg, about 8.75 mg/kg, about 9 mg/kg, about 9.25 mg/kg, about 9.5 mg/kg, about 9.75 mg/kg, about 10 mg/kg, about 10.25 mg/kg, about 10.5 mg/kg, about 10.75 mg/kg, about 11 mg/kg, about 11.25 mg/kg, about 11.5 mg/kg, about 11.75 mg/kg, about 12 mg/kg, about 12.25 mg/kg, about 12.5 mg/kg, about 12.75 mg/kg, about 13 mg/kg, about 13.25 mg/kg, about 13.5 mg/kg, about 13.75 mg/kg, about 14 mg/kg, about 14.25 mg/kg, about 14.25 mg/kg, about 14.5 mg/kg, about 14.75 mg/kg, about 15 mg/kg, about 15.25 mg/kg, about 15.5 mg/kg, about 15.75 mg/kg, about 16 mg/kg, about 16.25 mg/kg, about 16.5 mg/kg, about 16.75 mg/kg, about 17 mg/kg, about 17.25 mg/kg, about 17.5 mg/kg, about 17.75 mg/kg, about 18 mg/kg, about 18.25 mg/kg, about 18.5 mg/kg, about 18.75 mg/kg, about 19 mg/kg, about 19.25 mg/kg, about 19.5 mg/kg, about 19.75 mg/kg, or about 20 mg/kg.

In some aspects, the single dose of MEDI4736 is administered once weekly, once every two weeks, once every three weeks, once every four weeks, once every five weeks, or once every six weeks.

In some aspects, the PD-L1 antagonist is MPDL3280A. In some aspects, a therapeutically effective amount of MPDL3280A is administered to the subject. In some aspects, the MPDL3280A is administered at a single dose of about 750 mg to about 1500 mg. In some aspects, the MPDL3280A is administered at a single dose of about 750 mg, about 760 mg, about 770 mg, about 775 mg, about 780 mg, about 790 mg, about 800 mg, about 810 mg, about 820 mg, about 825 mg, about 830 mg, about 840 mg, about 850 mg, about 860 mg, about 870 mg, about 875 mg, about 880 mg, about 890 mg, about 900 mg, about 910 mg, about 920 mg, about 925 mg, about 930 mg, about 940 mg, about 950 mg, about 960 mg, about 970 mg, about 975 mg, about 980 mg, about 990 mg, about 1000 mg, about 1010 mg, about 1020 mg, about 1025 mg, about 1030 mg, about 1040 mg, about 1050 mg, about 1060 mg, about 1070 mg, about 1075 mg, about 1080 mg, about 1090 mg, about 1100 mg, about 1110 mg, about 1120 mg, about 1125 mg, about 1130 mg, about 1140 mg, about 1150 mg, about 1160 mg, about 1170 mg, about 1175 mg, about 1180 mg, about 1190 mg, about 1200 mg, about 1210 mg, about 1220 mg, about 1225 mg, about 1230 mg, about 1240 mg, about 1250 mg, about 1260 mg, about 1270 mg, about 1275 mg, about 1280 mg, about 1290 mg, about 1300 mg, about 1310 mg, about 1320 mg, about 1325 mg, about 1330 mg, about 1340 mg, about 1350 mg, about 1360 mg, about 1370 mg, about 1375 mg, about 1380 mg, about 1390 mg, about 1400 mg, about 1410 mg, about 1420 mg, about 1425 mg, about 1430 mg, about 1440 mg, about 1450 mg, about 1460 mg, about 1470 mg, about 1475 mg, about 1480 mg, about 1490 mg, or about 1500 mg.

In some aspects, the single dose of MPDL3280A is administered once weekly, once every two weeks, once every three weeks, once every four weeks, once every five weeks, or once every six weeks.

In some aspects, the PD-L1 antagonist is BMS-936559. In some aspects, a therapeutically effective amount of BMS-936559 is administered to the subject. In some aspects, the BMS-936559 is administered at a single dose of about 0.1 mg/kg to about 10 mg/kg. is administered at a single dose of about 0.1 mg/kg, about 0.25 mg/kg, about 0.5 mg/kg, about 0.75 mg/kg, about 1 mg/kg, about 1.25 mg/kg, about 1.5 mg/kg, about 1.75 mg/kg, about 2 mg/kg, about 2.25 mg/kg, about 2.5 mg/kg, about 2.75 mg/kg, about 3 mg/kg, about 3.25 mg/kg, about 3.5 mg/kg, about 3.75 mg/kg, about 4 mg/kg, about 4.25 mg/kg, about 4.5 mg/kg, about 4.75 mg/kg, about 5 mg/kg, about 5.25 mg/kg, about 5.5 mg/kg, about 5.75 mg/kg, about 6 mg/kg, about 6.25 mg/kg, about 6.5 mg/kg, about 6.75 mg/kg, about 7 mg/kg, about 7.25 mg/kg, about 7.5 mg/kg, about 7.75 mg/kg, about 8 mg/kg, about 8.25 mg/kg, about 8.5 mg/kg, about 8.75 mg/kg, about 9 mg/kg, about 9.25 mg/kg, about 9.5 mg/kg, about 9.75 mg/kg, or about 10 mg/kg.

In some aspects, the single dose of BMS-936559 is administered once weekly, once every two weeks, once every three weeks, once every four weeks, once every five weeks, or once every six weeks.

### CTLA-4 Antagonists

In some aspects, the one or more CTLA-4 antagonist is selected from the group consisting of ipilimumab, BMS-986218, AGEN1181, tremelimumab, and combinations thereof. In some aspects, a therapeutically effective amount of the one or more CTLA-4 antagonist is administered to the subject.

In some aspects, the CTLA-4 antagonist is ipilimumab. In some aspects, a therapeutically effective amount of ipilimumab is administered to the subject. In some aspects, the ipilimumab is administered at a single dose of about 0.25 mg/kg to about 15 mg/kg. In some aspects, the ipilimumab is administered at a single dose of about 0.25 mg/kg, about 0.5 mg/kg, about 0.75 mg/kg, about 1 mg/kg, about 1.25 mg/kg, about 1.5 mg/kg, about 1.75 mg/kg, about 2 mg/kg, about 2.25 mg/kg, about 2.5 mg/kg, about 2.75 mg/kg, about 3 mg/kg, about 3.25 mg/kg, about 3.5 mg/kg, about 3.75 mg/kg, about 4 mg/kg, about 4.25 mg/kg, about 4.5 mg/kg, about 4.75 mg/kg, about 5 mg/kg, about 5.25 mg/kg, about 5.5 mg/kg, about 5.75 mg/kg, about 6 mg/kg, about 6.25 mg/kg, about 6.5 mg/kg, about 6.75 mg/kg, about 7 mg/kg, about 7.25 mg/kg, about 7.5 mg/kg, about 7.75 mg/kg, about 8 mg/kg, about 8.25 mg/kg, about 8.5 mg/kg, about 8.75 mg/kg, about 9 mg/kg, about 9.25 mg/kg, about 9.5 mg/kg, about 9.75 mg/kg, about 10 mg/kg, about 10.25 mg/kg, about 10.5 mg/kg, about 10.75 mg/kg, about 11 mg/kg, about 11.25 mg/kg, about 11.5 mg/kg, about 11.75 mg/kg, about 12 mg/kg, about 12.25 mg/kg, about 12.5 mg/kg, about 12.75 mg/kg, about 13 mg/kg, about 13.25 mg/kg, about 13.5 mg/kg, about 13.75 mg/kg, about 14 mg/kg, about 14.25 mg/kg, about 14.25 mg/kg, about 14.5 mg/kg, about 14.75 mg/kg, or about 15 mg/kg.

In some aspects, the single dose of ipilimumab is administered once weekly, once every two weeks, once every three weeks, once every four weeks, once every five weeks, once every six weeks, once every seven weeks, once every eight weeks, once every nine weeks once every ten weeks, once every 11 weeks, or once every 12 weeks. In some aspects, the single dose of ipilimumab is administered once every three weeks. In some aspects, the single dose of ipilimumab is administered once every 12 weeks.

In some aspects, the single dose of ipilimumab is about 1 mg/kg administered once every three weeks. In some aspects, the single dose of ipilimumab is about 3 mg/kg administered once every three weeks. In some aspects, the single dose of ipilimumab is about 10 mg/kg administered once every three weeks. In some aspects, the single dose of ipilimumab is about 10 mg/kg administered once every 12 weeks.

In some aspects, the CTLA-4 antagonist is BMS-986218. In some aspects, a therapeutically effective amount of BMS-986218 is administered to the subject. In some aspects, the BMS-986218 is administered in a single dose of about 0.5 mg to about 100 mg. In some aspects, the BMS-986218 is administered at a single dose of about 0.5 mg, about 0.75 mg, about 1 mg, about 1.25 mg, about 1.5 mg, about 1.75 mg, about 2 mg, about 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, about 5 mg, about 5.25 mg, about 5.50 mg, about 5.75 mg, about 6 mg, about 6.50 mg, about 6.75 mg, about 7 mg, about 7.25 mg, about 7.5 mg, about 8 mg, about 8.25 mg, about 8.5 mg, about 8.75 mg, about 9 mg, about 9.25 mg, about 9.5 mg, about 9.75 mg, about 10 mg, about 10.25 mg, about 10.5 mg, about 10.75 mg, about 11 mg, about 11.25 mg, about 11.5 mg, about 11.75 mg, about 12 mg, about 12.5 mg, about 13 mg, about 13.5 mg, about 14 mg, about 14.5 mg, about 15 mg, about 15.5 mg, about 16 mg, about 16.5 mg, about 17 mg, about 17.5 mg, about 18 mg, about 18.5 mg, about 19 mg, about 19.5 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, or about 100 mg.

In some aspects, the single dose of BMS-986218 is administered once weekly, once every two weeks, once every three weeks, once every four weeks, once every five weeks, once every six weeks, once every seven weeks, once every eight weeks, once every nine weeks once every ten weeks, once every 11 weeks, or once every 12 weeks. In some aspects, the single dose of BMS-986218 is administered once every four weeks.

In some aspects, the CTLA-4 antagonist is AGEN1181. In some aspects, a therapeutically effective amount of AGEN1181 is administered to the subject. In some aspects, the AGEN1181 is administered at a single dose of about 0.05 mg/kg to about 10 mg/kg. In some aspects, the AGEN1181 is administered at a single dose of about 0.05 mg/kg, about 0.1 mg/kg, about 0.2 mg/kg, about 0.25 mg/kg, about 0.3 mg/kg, about 0.4 mg/kg, about 0.5 mg/kg, about 0.6 mg/kg, about 0.7 mg/kg, about 0.75 mg/kg, about 0.8 mg/kg, about 0.9 mg/kg, about 1 mg/kg, about 1.25 mg/kg, about 1.5 mg/kg, about 1.75 mg/kg, about 2 mg/kg, about 2.25 mg/kg, about 2.5 mg/kg, about 2.75 mg/kg, about 3 mg/kg, about 3.25 mg/kg, about 3.5 mg/kg, about 3.75 mg/kg, about 4 mg/kg, about 4.25 mg/kg, about 4.5 mg/kg, about 4.75 mg/kg, about 5 mg/kg, about 5.25 mg/kg, about 5.5 mg/kg, about 5.75 mg/kg, about 6 mg/kg, about 6.25 mg/kg, about 6.5 mg/kg, about 6.75 mg/kg, about 7 mg/kg, about 7.25 mg/kg, about 7.5 mg/kg, about 7.75 mg/kg, about 8 mg/kg, about 8.25 mg/kg, about 8.5 mg/kg, about 8.75 mg/kg, about 9 mg/kg, about 9.25 mg/kg, about 9.5 mg/kg, about 9.75 mg/kg, or about 10 mg/kg.

In some aspects, the single dose of AGEN1181 is administered once weekly, once every two weeks, once every three weeks, once every four weeks, once every five weeks, once every six weeks, once every seven weeks, once every eight weeks, once every nine weeks once every ten weeks, once every 11 weeks, or once every 12 weeks. In some aspects, the single dose of AGEN1181 is administered once every three weeks. In some aspects, the single dose of AGEN1181 is administered once every six weeks.

In some aspects, the single dose of AGEN1181 is about 0.1 mg/kg to about 4 mg/kg administered once every three weeks. In some aspects, the single dose of AGEN1181 is about 1 mg/kg to about 4 mg/kg administered once every six weeks.

In some aspects, the CTLA-4 antagonist is tremelimumab. In some aspects, a therapeutically effective amount of tremelimumab is administered to the subject. In some aspects, the tremelimumab is administered as a single dose of about 1 mg/kg to about 15 mg/kg. is administered at a single dose of about 1 mg/kg, about 1.25 mg/kg, about 1.5 mg/kg, about 1.75 mg/kg, about 2 mg/kg, about 2.25 mg/kg, about 2.5 mg/kg, about 2.75 mg/kg, about 3 mg/kg, about 3.25 mg/kg, about 3.5 mg/kg, about 3.75 mg/kg, about 4 mg/kg, about 4.25 mg/kg, about 4.5 mg/kg, about 4.75 mg/kg, about 5 mg/kg, about 5.25 mg/kg, about 5.5 mg/kg, about 5.75 mg/kg, about 6 mg/kg, about 6.25 mg/kg, about 6.5 mg/kg, about 6.75 mg/kg, about 7 mg/kg, about 7.25 mg/kg, about 7.5 mg/kg, about 7.75 mg/kg, about 8 mg/kg, about 8.25 mg/kg, about 8.5 mg/kg, about 8.75 mg/kg, about 9 mg/kg, about 9.25 mg/kg, about 9.5 mg/kg, about 9.75 mg/kg, about 10 mg/kg, about 10.25 mg/kg, about 10.5 mg/kg, about 10.75 mg/kg, about 11 mg/kg, about 11.25 mg/kg, about 11.5 mg/kg, about 11.75 mg/kg, about 12 mg/kg, about 12.25 mg/kg, about 12.5 mg/kg, about 12.75 mg/kg, about 13 mg/kg, about 13.25 mg/kg, about 13.5 mg/kg, about 13.75 mg/kg, about 14 mg/kg, about 14.25 mg/kg, about 14.25 mg/kg, about 14.5 mg/kg, about 14.75 mg/kg, or about 15 mg/kg.

In some aspects, the single dose of tremelimumab is administered once weekly, once every two weeks, once every three weeks, once every four weeks, once every five weeks, once every six weeks, once every seven weeks, once every eight weeks, once every nine weeks once every ten weeks, once every 11 weeks, or once every 12 weeks.

All of the references cited above, as well as all references cited herein, are incorporated herein by reference in their entireties.

Any examples provided herein are offered by way of illustration and not by way of limitation.

### EXAMPLES

### Example 1: GR Activates PD-L1 Expression and Represses MHC-I Expression in PDAC Cells

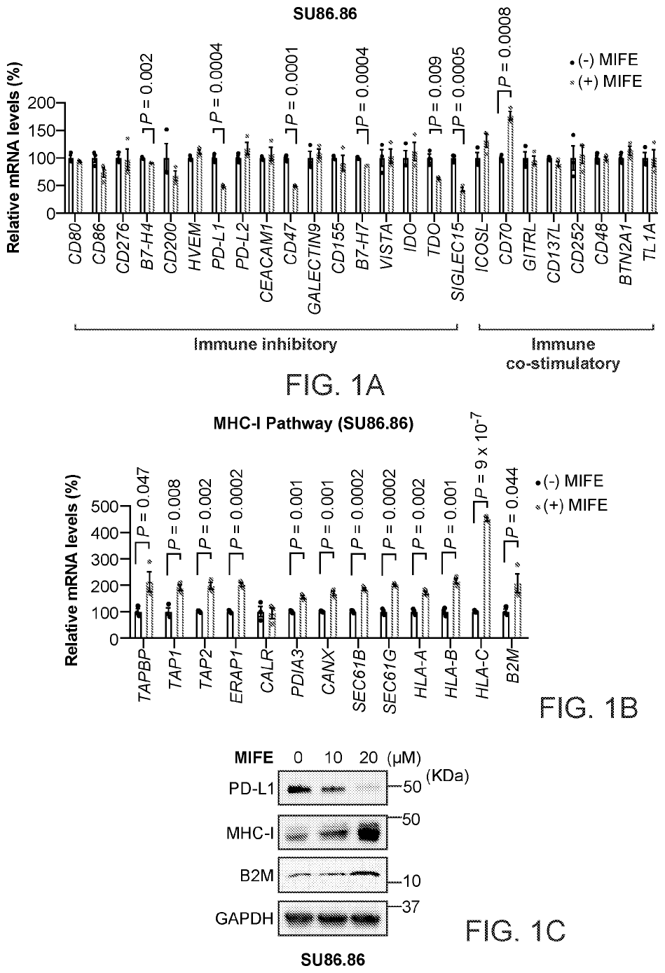

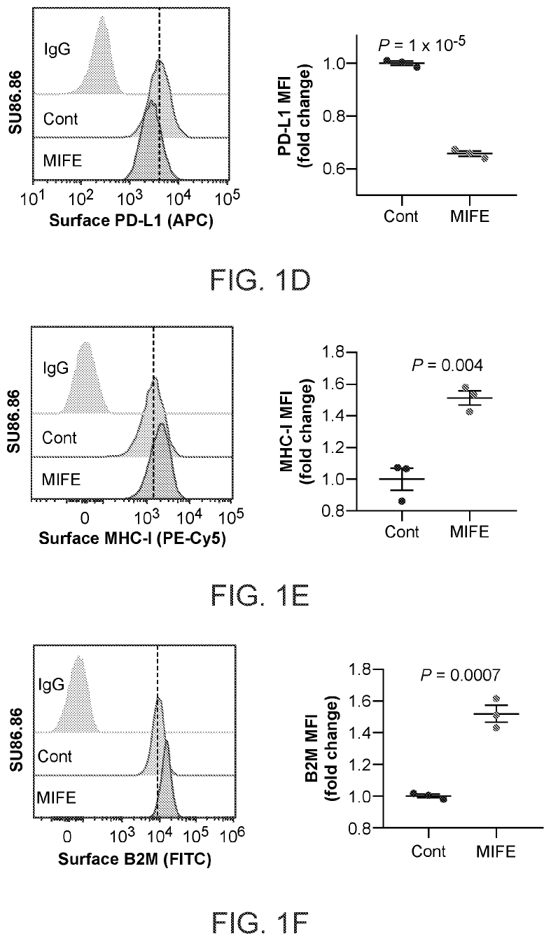

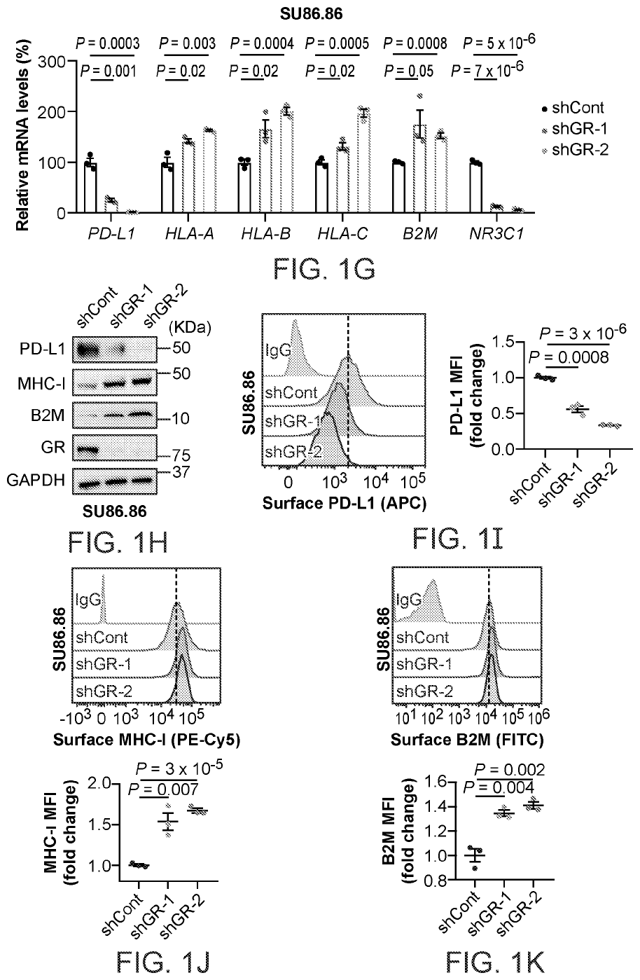

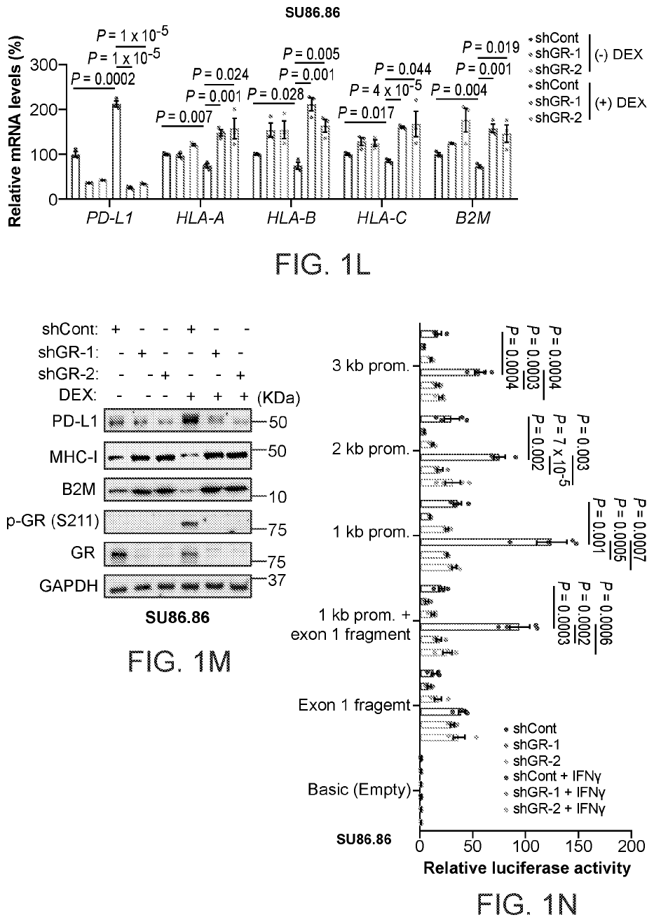

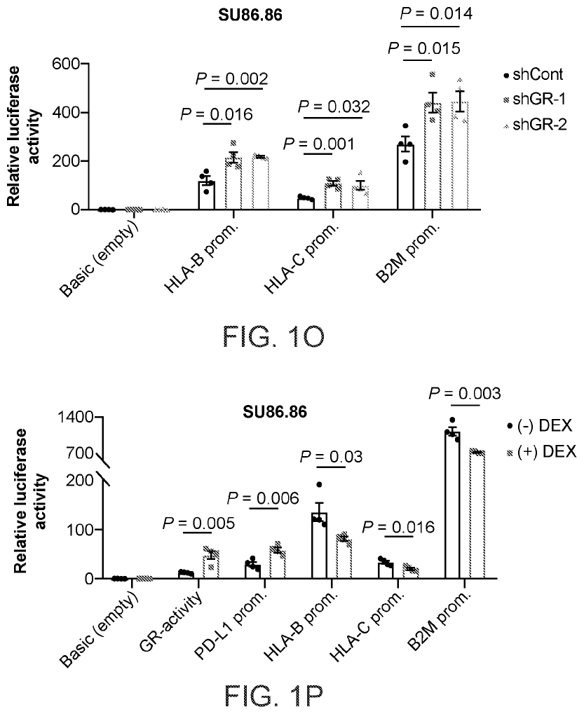

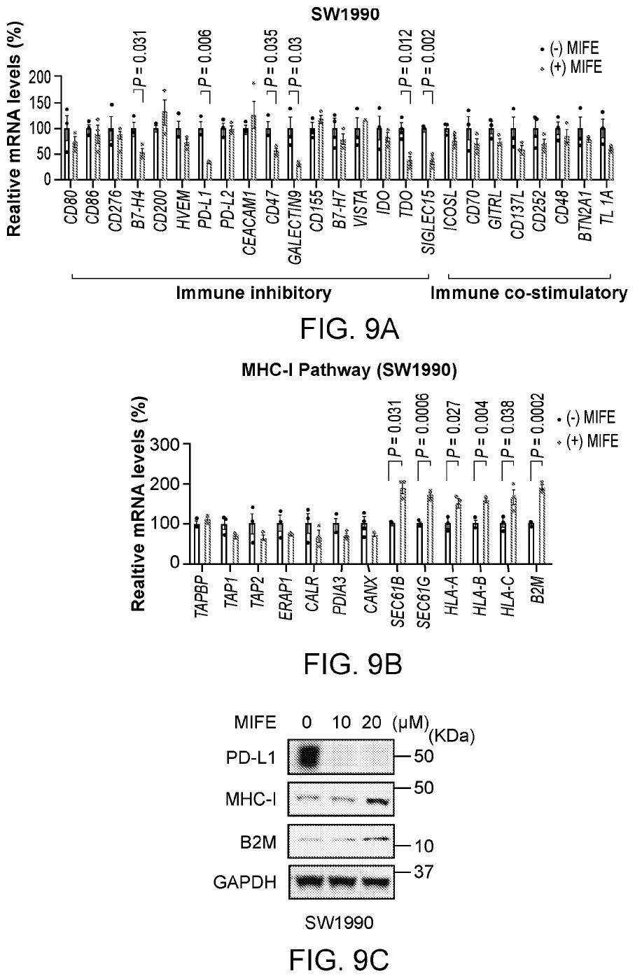

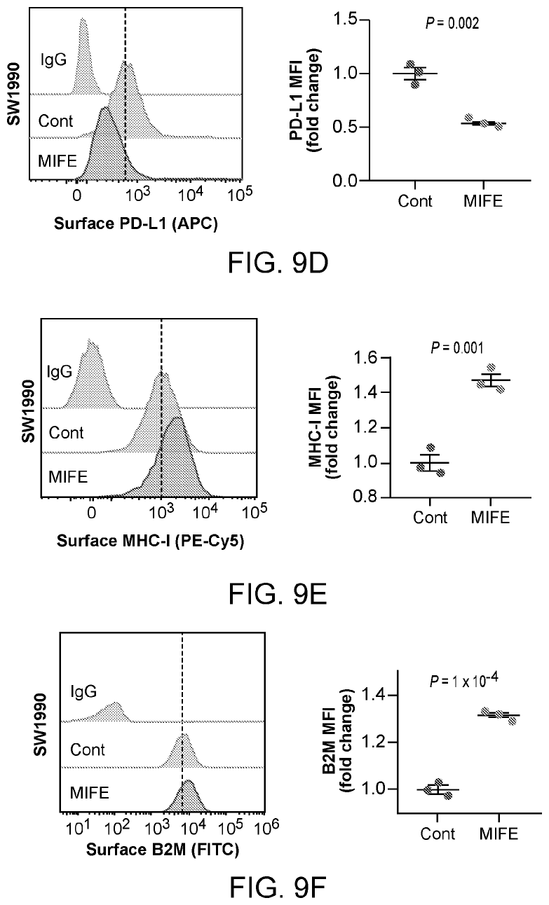

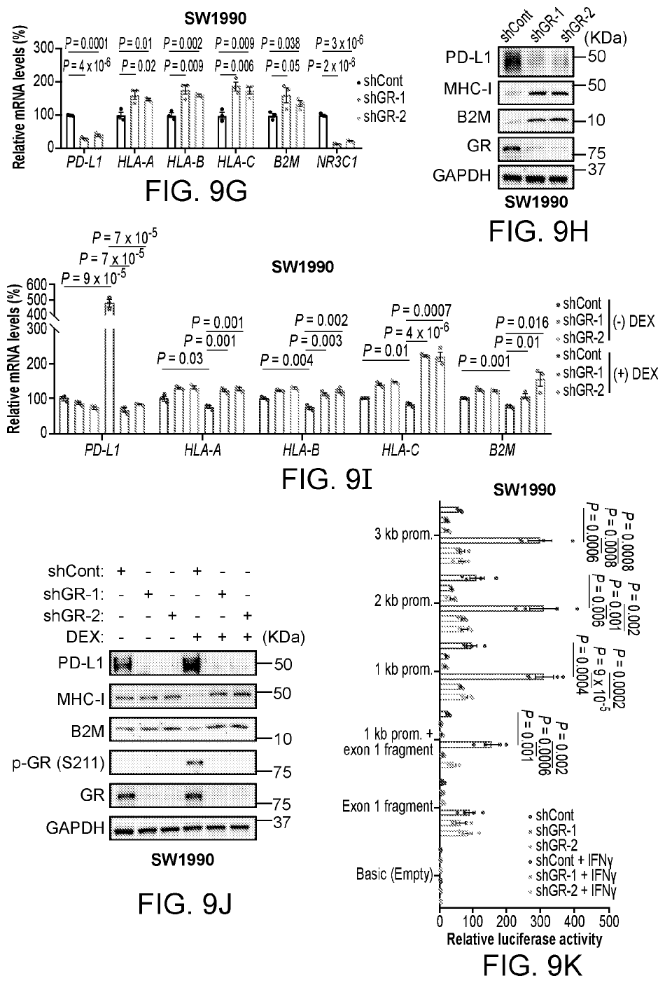

To determine whether GR regulates immunity-related genes in pancreatic cancer cells, two human PDAC cell lines harboring the G12D hotspot mutation of KRAS, as shown in Deer, E. L., et al. Phenotype and genotype of pancreatic cancer cell lines. Pancreas 39, 425-435 (2010) and Dutil, J., et al., An Interactive Resource to Probe Genetic Diversity and Estimated Ancestry in Cancer Cell Lines. Cancer Res 79, 1263-1273 (2019). SU86.86 (female) and SW1990 (male) were treated with the clinical GR antagonist mifepristone (also known as RU486; used to treat patients with Cushing's syndrome characterized by aberrantly high levels of glucocorticoids). qPCR analysis revealed that mifepristone treatment of both cell lines decreased mRNA levels of several immune checkpoint ligands, including PD-L1, CD47, TDO, and SIGLEC15 (FIG. 1A and FIG. 9A), and increased mRNA levels of several components in the major histocompatibility complex class I (MHC-I) pathway, including HLA-A, HLA-B, HLA-C, B2M, SEC61B, and SEC61G (FIG. 1B and FIG. 9B). These targets have been previously described in Burugu, S., Dancsok, A. R. & Nielsen, T. O. Emerging targets in cancer immunotherapy. Semin Cancer Biol 52, 39-52 (2018). Consistent with the effect on mRNA, PD-L1 protein was also downregulated by mifepristone treatment, as gauged by Western blot analysis (FIG. 1C and FIG. 9C) and flow cytometric analysis (FIG. 1D and FIG. 9D). Similarly, upregulation of MHC-I and β-2-microglobulin (B2M, the common light chain of the MHC complex) proteins was observed in both SU86.86 and SW1990 cell lines treated with the GR antagonist (FIG. 1C, FIG. 1E, and FIG. 1F; and FIG. 9C, FIG. 9E, and FIG. 9F).

To further assess the role of GR in regulating PD-L1 and MHC-I expression levels in pancreatic cancer cells, GR was knocked down in SU86.86 and SW1990 cell lines by two independent shRNAs, finding that silencing of GR significantly downregulated PD-L1 and upregulated MHC-I and B2M at both mRNA and protein levels (FIGS. 1G-1K, FIG. 9G, and FIG. 9H). Treatment of both PDAC cell lines with a clinical GR agonist, dexamethasone (DEX), led to upregulation of PD-L1 and downregulation of MHC-I and B2M, which could be reversed by knockdown of GR (FIGS. 1L-1M, FIGS. 91-9J).

GR can either activate or repress gene transcription. Reviewed in Cain, D. W. & Cidlowski, J. A. Immune regulation by glucocorticoids. Nat Rev Immunol 17, 233-247 (2017). Thus, whether GR regulates the transcription of PD-L1 (encoded by (I) 274) and MHC-I genes was investigated. By using a series of luciferase reporter constructs containing previously described promoter fragments cloned from the human (I) 274 gene, it was found that IFNγ (known to activate PD-L1 transcription) induced the activity of the promoter reporters in both SU86.86 and SW1990 cells, which was abrogated by knockdown of GR (FIG. 1N and FIG. 9K). Coelho, M. A., et al. Oncogenic RAS Signaling Promotes Tumor Immunoresistance by Stabilizing PD-L1 mRNA. Immunity 47, 1083-1099 e1086 (2017).

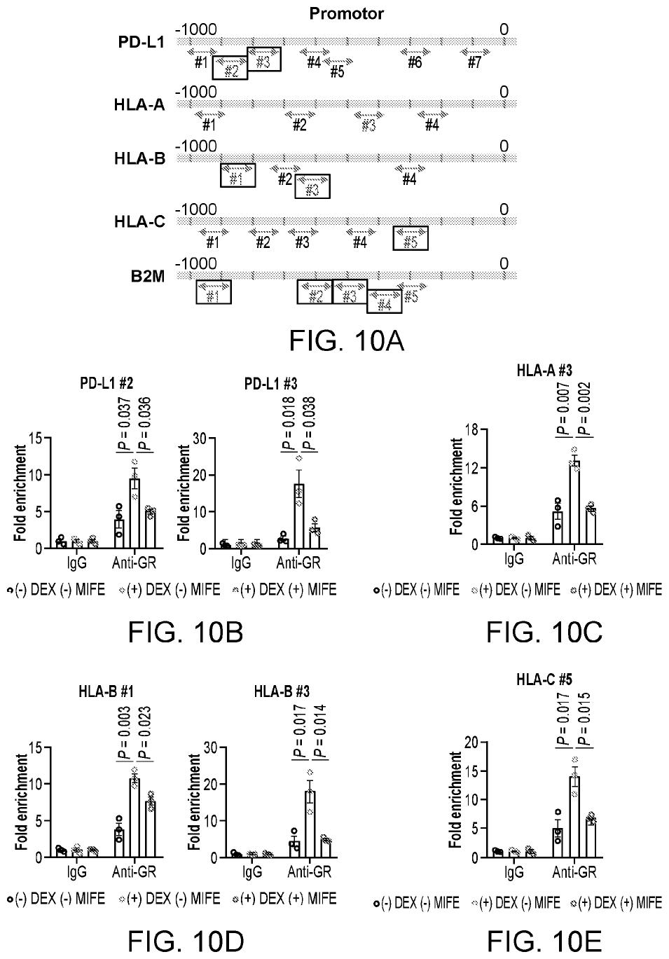

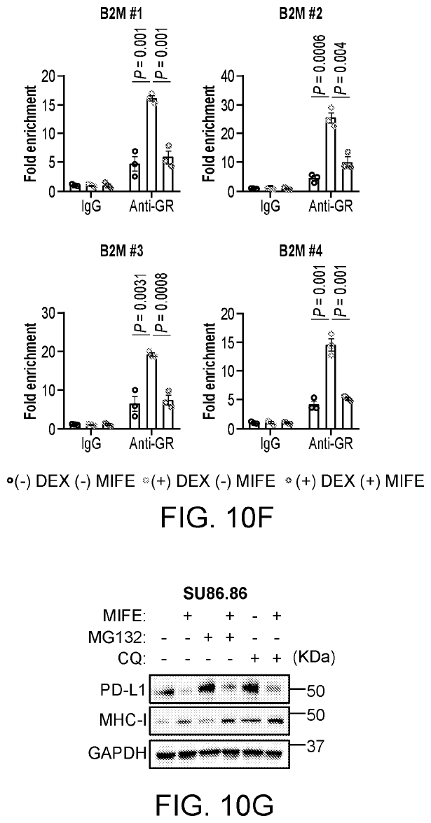

Moreover, silencing of GR upregulated, and dexamethasone treatment downregulated, the activity of the luciferase reporter containing the promoter of HLA-B, HLA-C, or B2M (FIG. 1O and FIG. 1P). In addition, the promoter regions of PD-L1, HLA-A, HLA-B, HLA-C, and B2M genes were analyzed and identified multiple glucocorticoid response elements (GREs). PCR amplicons were then designed for genomic regions encompassing these putative GR-binding sites (FIG. 10A). ChIP-qPCR analysis of SU86.86 cells revealed that for each of the five genes, at least one predicted binding site met the following criteria: the binding to GR was significantly induced by dexamethasone treatment, which was reversed by co-treatment with mifepristone (FIG. 10B-10F). Treatment with the proteasome inhibitor MG132 or the lysosome inhibitor chloroquine did not affect mifepristone-mediated PD-L1 downregulation and MHC-I upregulation (FIG. 10G), indicating that GR regulates PD-L1 and MHC-I expression independently of the proteasomal or lysosomal pathway. Collectively, these results provide evidence for the direct transcriptional regulation of PD-L1 and MHC-I genes by GR.

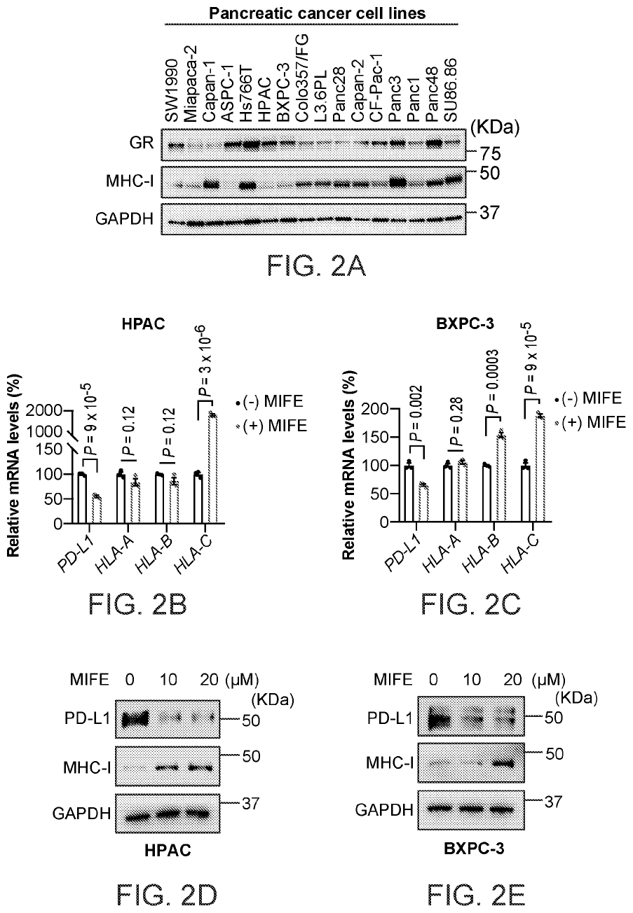

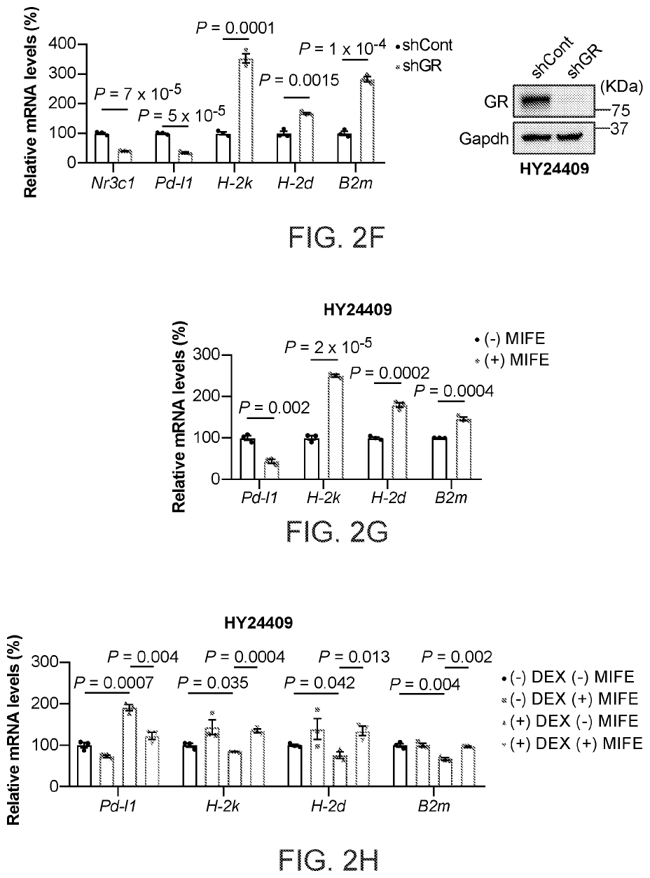

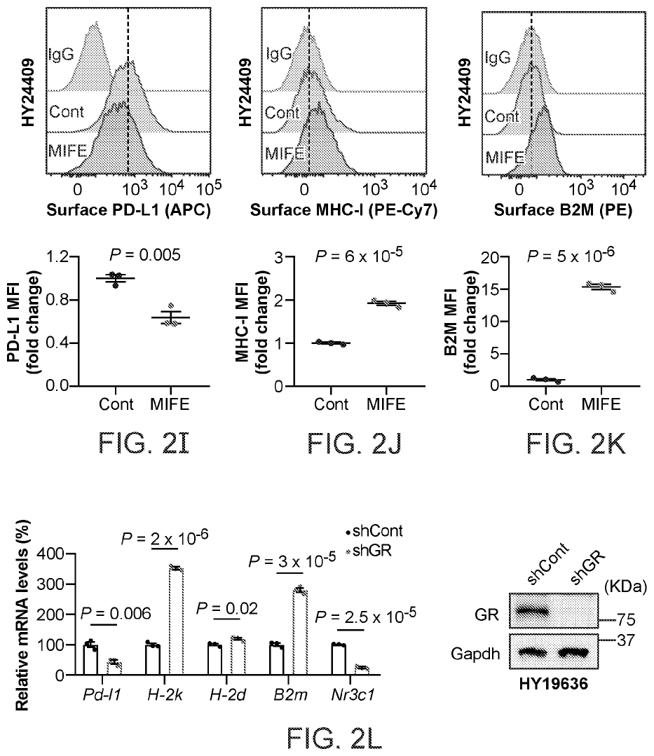

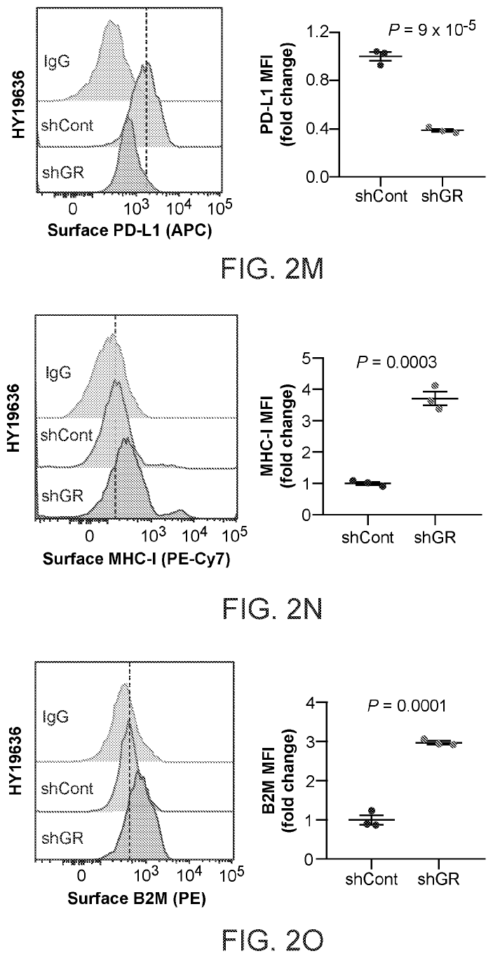

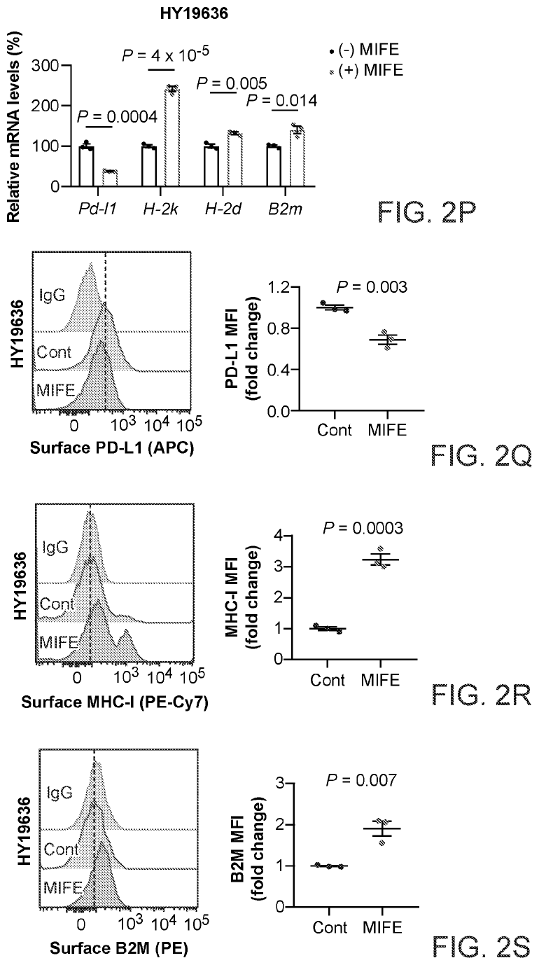

To further determine whether modulation of PD-L1 and MHC-I by GR is a general regulatory mechanism in PDAC, MHC-I and GR protein levels in 16 human PDAC lines were examined. HPAC (female) and BXPC-3 (female) cell lines showed high GR expression and low MHC-I expression (FIG. 2A). Mifepristone treatment of HPAC and BXPC-3 cells significantly upregulated MHC-I and downregulated PD-L1 at both mRNA and protein levels (FIG. 2B-2E), showing that the GR antagonist increased MHC-I expression in PDAC cell lines with low MHC-I expression. Consistent with the results from human PDAC cell lines, knockdown of GR in a male mouse PDAC cell line HY24409 reduced PD-L1 mRNA levels and elevated mRNA levels of H-2k, H-2d, and B2m (FIG. 2F). Moreover, mifepristone treatment of HY24409 cells led to a decrease in PD-L1 mRNA levels and an increase in MHC-I and B2M mRNA levels, whereas dexamethasone treatment showed the opposite effect (FIGS. 2G-2H). Similarly, mifepristone treatment decreased surface PD-L1 levels and increased surface protein levels of MHC-I (H-2K^{b}) and B2M (FIGS. 2I-2K). In addition, in a female mouse PDAC cell line HY19636, knockdown of GR (FIGS. 2L-2O) or mifepristone treatment (FIGS. 2P-2S) downregulated PD-L1 and upregulated MHC-I and B2M at mRNA and surface protein levels. Taken together, these results suggest that GR activates PD-L1 expression and represses MHC-I expression in human and mouse PDAC cells in general, regardless of sex.

### Example 2: Tumor Cell-Specific GR Depletion or Pharmacologic GR Inhibition Promotes Anti-Tumor Immunity in PDAC

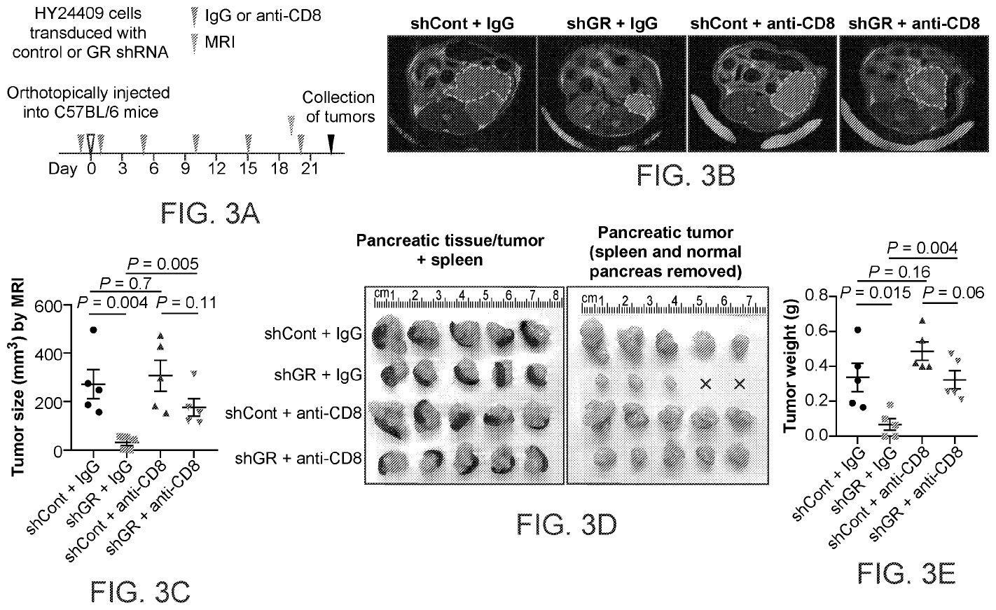

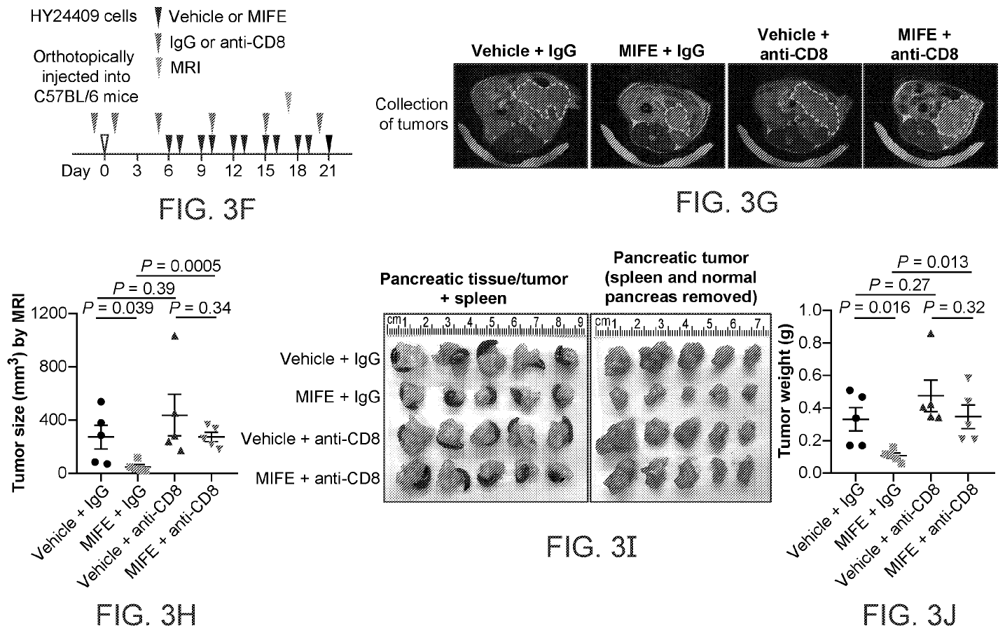

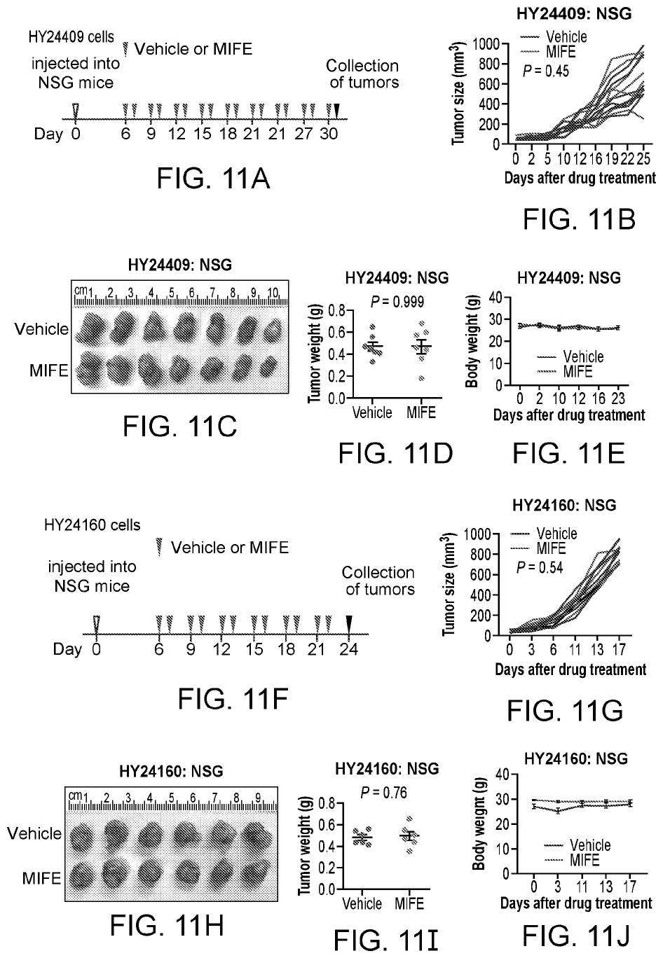

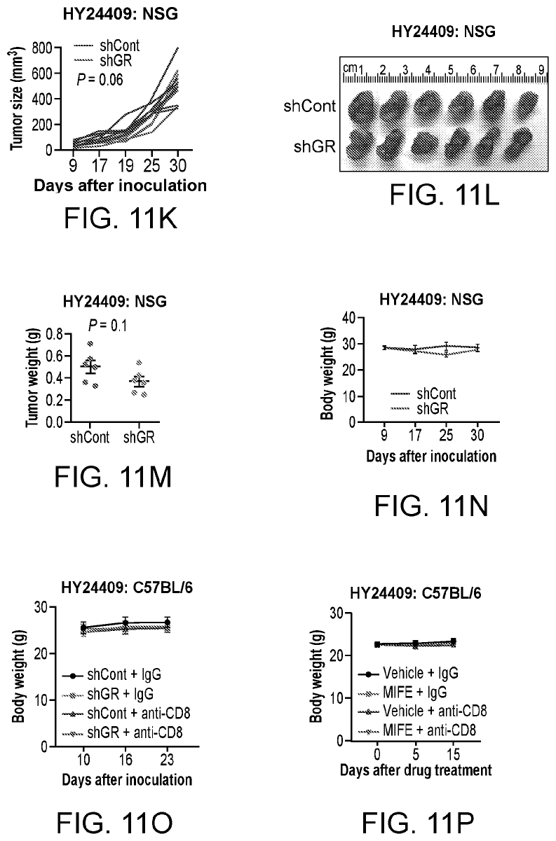

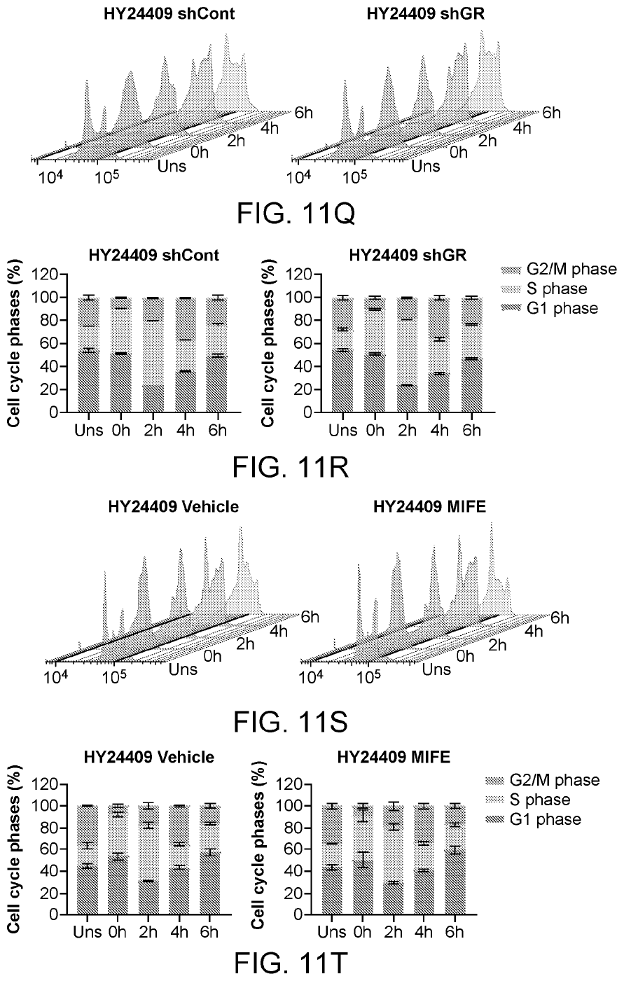

To determine the functional role of GR in pancreatic cancer cells, either NSG (non-obese diabetic; severe combined immunodeficiency; interleukin-2 receptor gamma chain null) mice or immunocompetent C57BL/6 mice were implanted with two mouse PDAC cell lines (HY24409 and HY24160), both of which were derived from male KPC (p48-Cre;Kras^{LSL-G12D/+}; Trp53^{loxP/+}) mice. Bardeesy, N., et al. Both p16 (Ink4a) and the p19 (Arf)-p53 pathway constrain progression of pancreatic adenocarcinoma in the mouse. Proc Natl Acad Sci USA 103, 5947-5952 (2006); Yamamoto, K., et al. Autophagy promotes immune evasion of pancreatic cancer by HC-I. Nature 581, 100-105 (2020). KPC mice were backcrossed to the C57BL/6 background (the purity of the C57BL/6 background of the mice used for KPC cell line generation was approximately 98% based on SNP analysis). The mice were then treated with mifepristone via oral gavage (60 mg kg-1, twice every 3 days). In NSG mice, neither mifepristone treatment (FIGS. 11A-11J) nor shRNA-mediated GR knockdown (FIGS. 11K-11N) affected the growth of tumors formed by KPC cell lines. In contrast, in C57BL/6 mice, either GR knockdown in HY24409 cells (FIG. 3A-3E) or systemic mifepristone treatment (FIGS. 3F-3J) led to substantial reductions in orthotopic pancreatic tumor volume (gauged by magnetic resonance imaging) and weight, without significant loss of body weight (FIGS. 11O-11P). At the endpoint, 80% and 67.7% reductions were observed in tumor weight by GR knockdown (FIG. 3D-3E) and mifepristone treatment (FIG. 3I-3J), respectively. Flow cytometric analysis of HY24409 cells collected from unsynchronized conditions and at different time points after release from double thymidine block (which arrests most cells at G1/S boundary, prior to DNA replication) showed that neither GR knockdown (FIGS. 11Q-11R) nor mifepristone treatment (FIGS. 11S-11T) altered the cell cycle profile. Altogether, these data indicate that the tumor microenvironment (TME), mostly likely the immune components, are involved in the observed tumor growth inhibition.

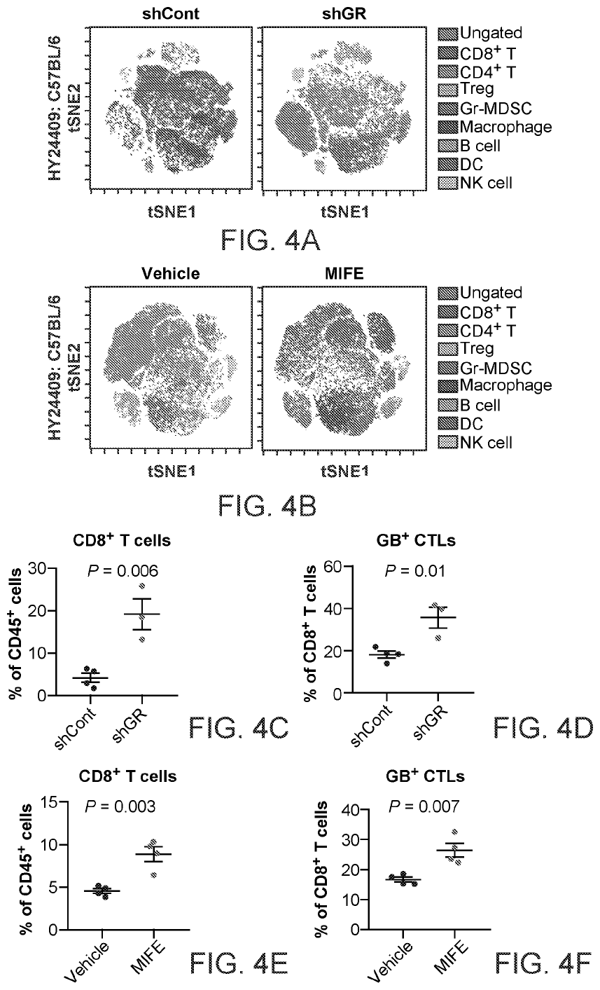

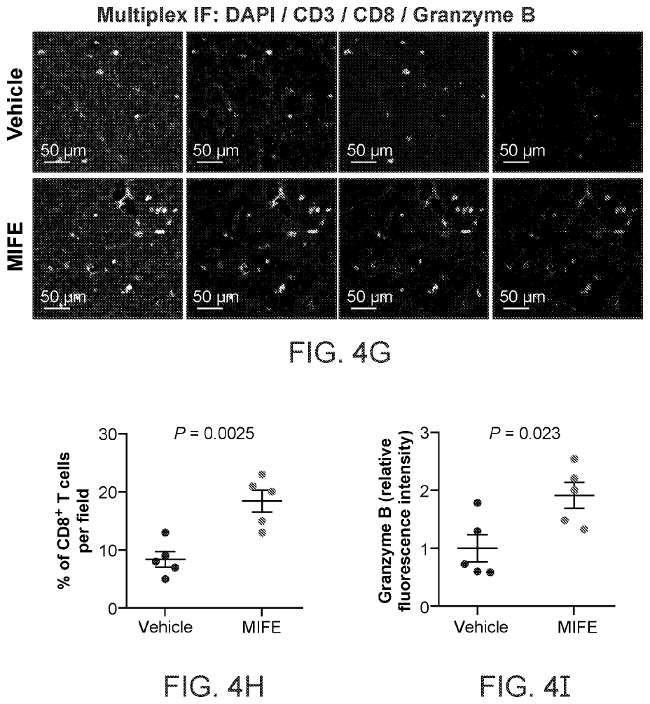

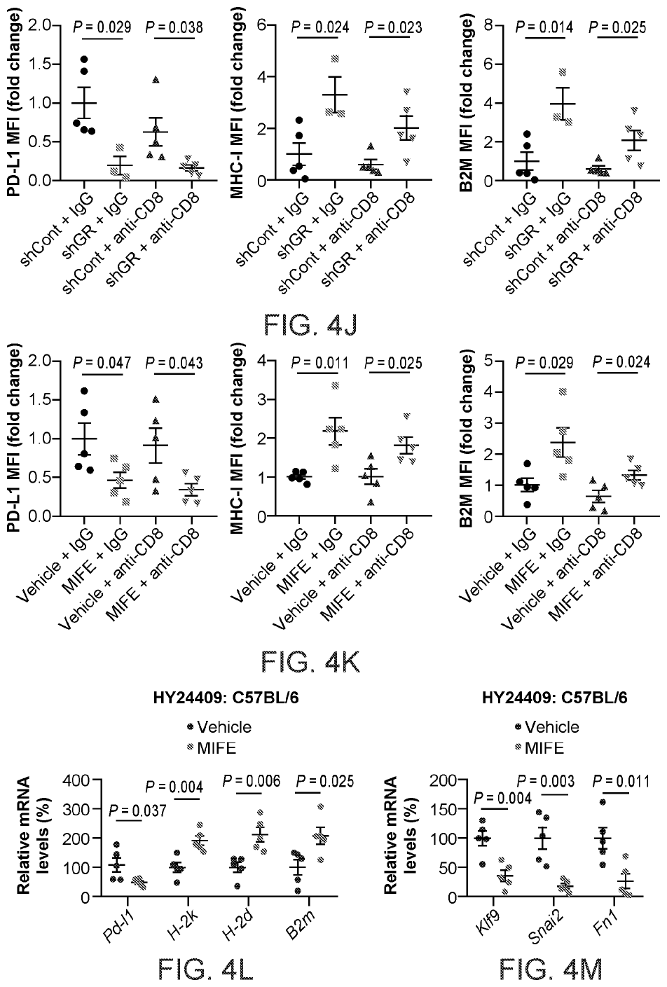

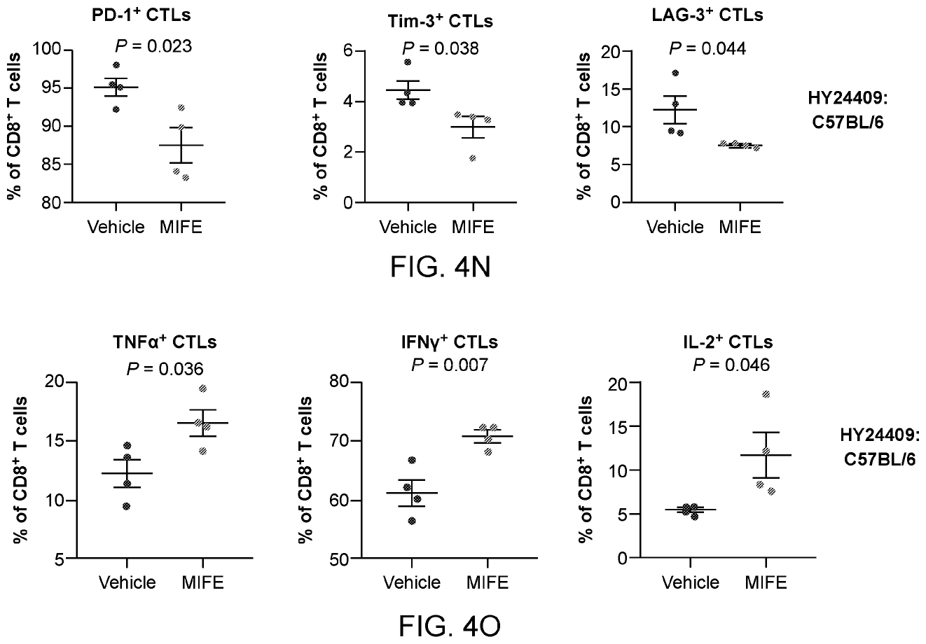

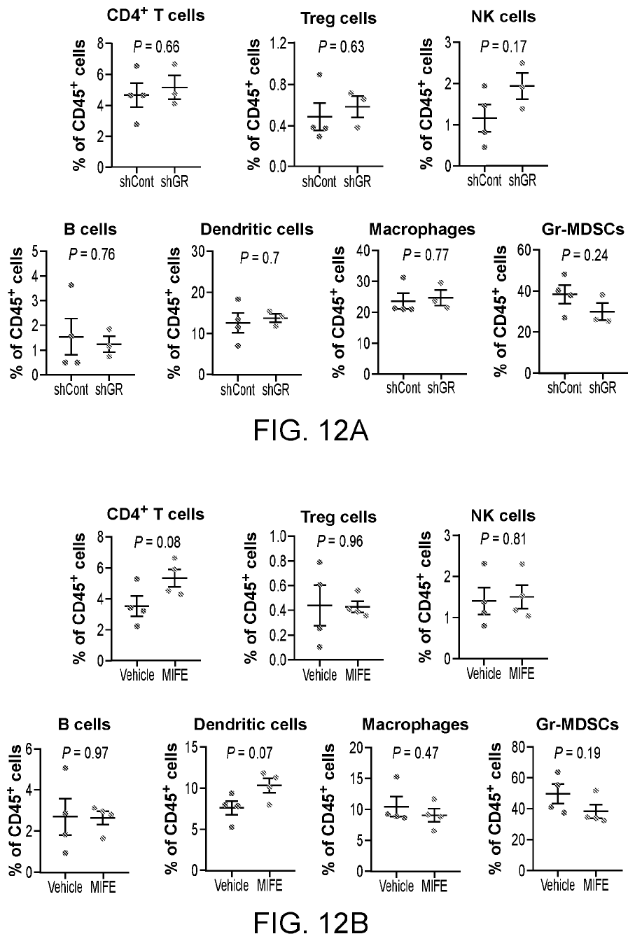

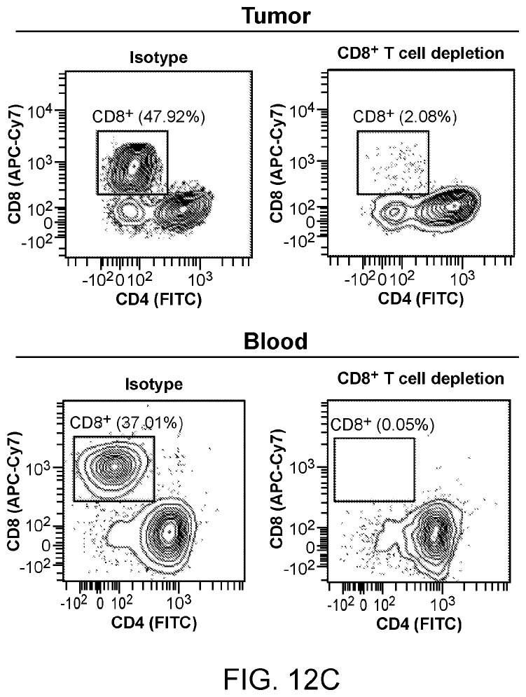

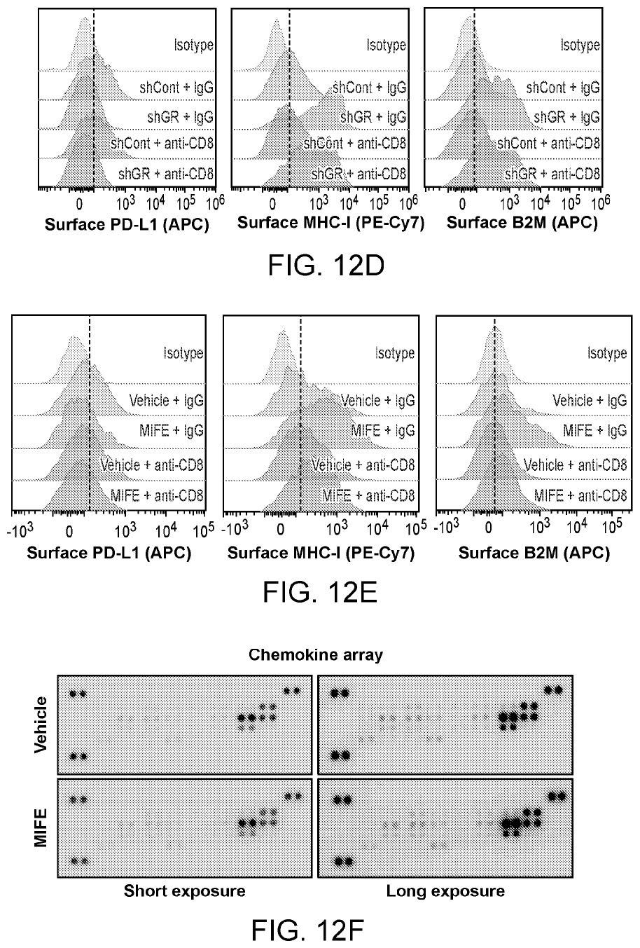

To assess the effect of tumor cell-specific GR depletion or pharmacologic GR inhibition on the tumor immune microenvironment, time-of-flight mass cytometry (CyTOF) was used for high-dimensional analysis of tumor-associated immune cells at the single-cell level (FIGS. 4A-4B). Bandura, D. R., et al. Mass cytometry: technique for real time single cell multitarget immunoassay based on inductively coupled plasma time-of-flight mass spectrometry. Anal Chem 81, 6813-6822 (2009); Bendall, S. C., et al. Single-cell mass cytometry of differential immune and drug responses across a human hematopoietic continuum. Science 332, 687-696 (2011). This analysis revealed that in pancreatic tumors generated by HY24409 cells, either GR knockdown or mifepristone treatment significantly increased the abundance and activity of CTLs, as gauged by CD8 (a marker of CTLs) and granzyme B (a marker of CTL activity), respectively (FIGS. 4A-4F). Specifically, knockdown of GR increased the percentages of tumor-infiltrating CD8+ cells and granzyme B+ CTLs by 4.6-fold and 2-fold (FIGS. 4C-4D), while treatment with mifepristone increased the percentages of tumor-infiltrating CD8+ cells and granzyme B+ CTLs by 1.9-fold and 1.6-fold (FIGS. 4E-F). No significant change was found in the abundance of CD4+ T cells, regulatory T cells, natural killer cells, B cells, dendritic cells, macrophages, and myeloid-derived suppressor cells (FIGS. 12A-12B). Multiplex immunofluorescent staining of CD3 (a marker of T cells), CD8, and granzyme B was performed in parallel, finding a substantial enrichment of these three markers in pancreatic tumors from mifepristone-treated C57BL/6 mice (FIGS. 4G-4I), further confirming the increase in tumor infiltration by active CTLs. Notably, antibody-mediated depletion of CD8+ T cells (FIG. 12C) abolished the tumor growth inhibition caused by GR knockdown (FIGS. 3B-3E) or mifepristone treatment (FIG. 3G-J), suggesting that CTLs mediate the observed anti-tumor effect of GR depletion or inhibition.

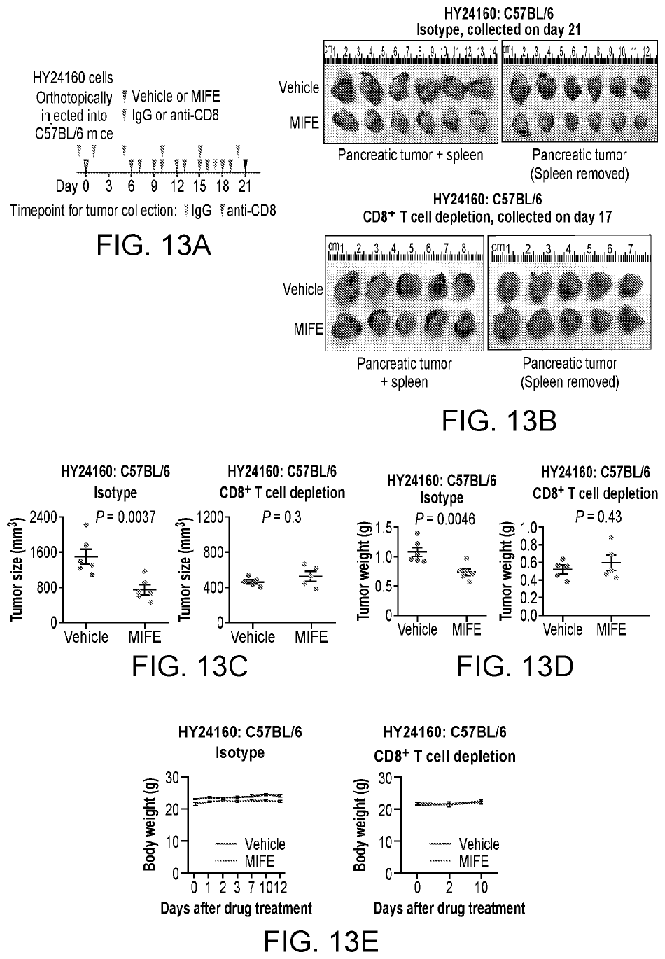

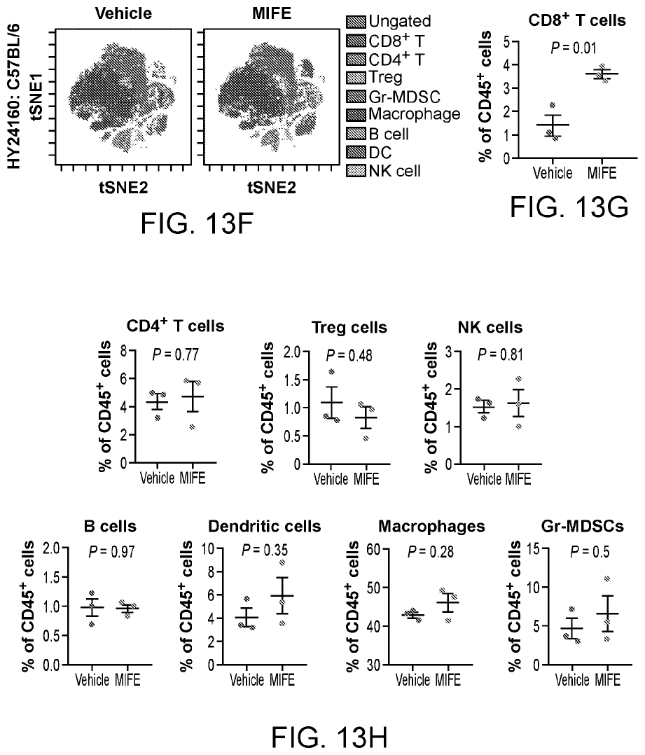

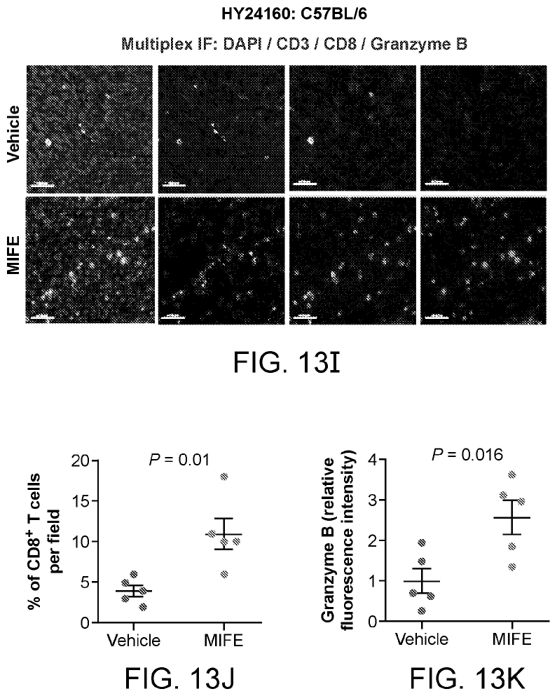

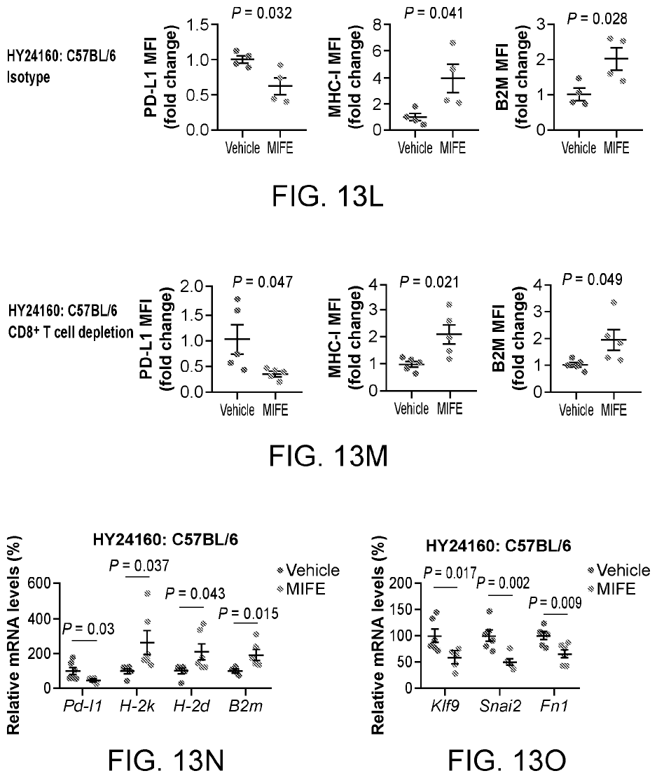

Consistent with the observed anti-tumor effect of GR knockdown or inhibition in immunocompetent mice shown herein, either GR-depleted or mifepristone-treated pancreatic tumors showed lower cell-surface PD-L1 levels as well as higher cell-surface MHC-I (H-2K^{b}) and B2M levels, as gauged by flow cytometric analysis (FIGS. 4J-4K and FIGS. 12D-12E). Moreover, mRNA levels of PD-L1 and known GR-activated target genes (Klf9, Snai2, and Fn1) were downregulated in mifepristone-treated tumors (FIGS. 4L-4M), whereas mRNA levels of MHC-I components (H-2k, H-2d, and B2m) were upregulated (FIG. 4L). Next, chemokines secreted by HY24409 cells were profiled. Several chemokines, including CXCL16, KC, LIX, and MIP-2, were present at high levels in the conditioned medium of HY24409 cells; however, no chemokines that showed a change after mifepristone treatment were found (FIG. 12F). PD-1, Tim-3, and LAG-3 were also examined by flow cytometry, finding a significant reduction in surface levels of these T cell exhaustion markers in mifepristone-treated HY24409 pancreatic tumors (FIG. 4N). Furthermore, flow cytometric analysis of intracellular TNFα, IFNγ, and IL-2 on tumor-associated CD8+ cells after ex vivo stimulation with phorbol myristate acetate (PMA) and ionomycin was performed. Mifepristone treatment significantly increased expression levels of these three cytokines (FIG. 4O). Taken together, these data suggest that GR inhibition elevates the number and activity of tumor-infiltrating CD8+ T cells. Similar to the results from the HY24409 model, in orthotopic pancreatic tumors formed by the HY24160 cell line, treatment with mifepristone also inhibited tumor growth (without affecting body weight) in a CTL-dependent manner (FIGS. 13A-13E), promoted the infiltration and activity of cytotoxic T cells (FIGS. 13F-13K), downregulated expression levels of PD-L1 and known GR-activated genes (FIGS. 13L-130), and upregulated MHC-I expression levels (FIGS. 13L-13N). These data suggest that GR promotes pancreatic cancer immune evasion.

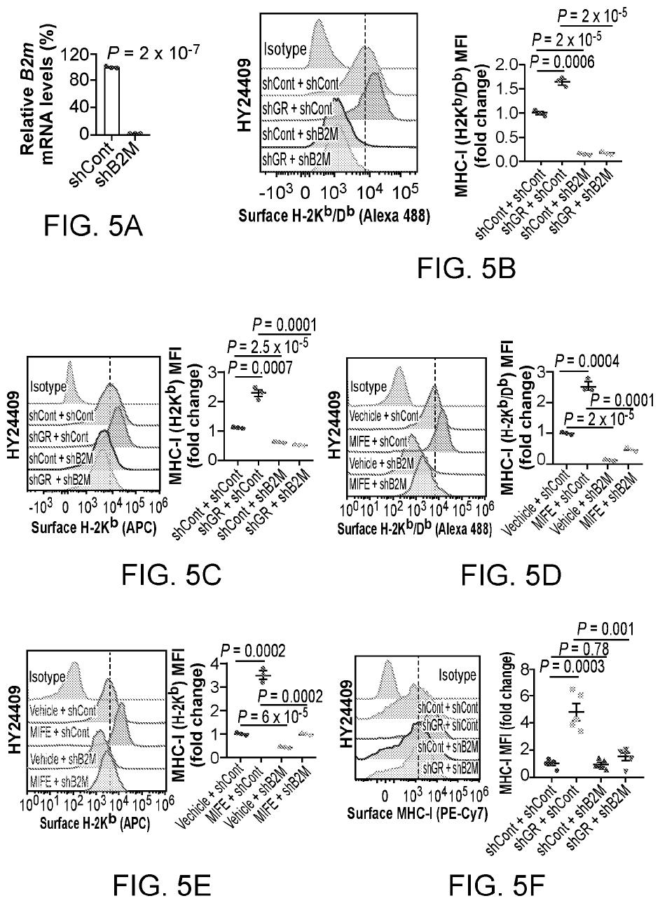

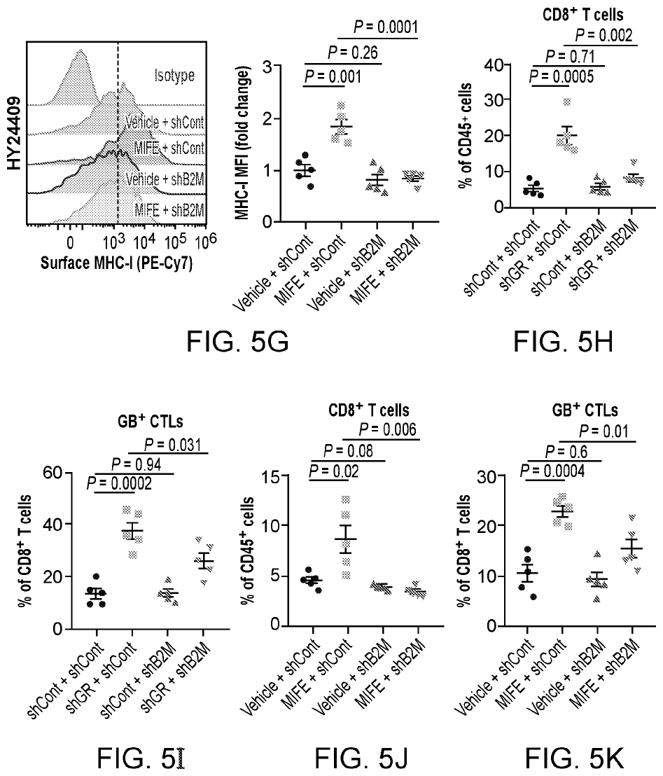

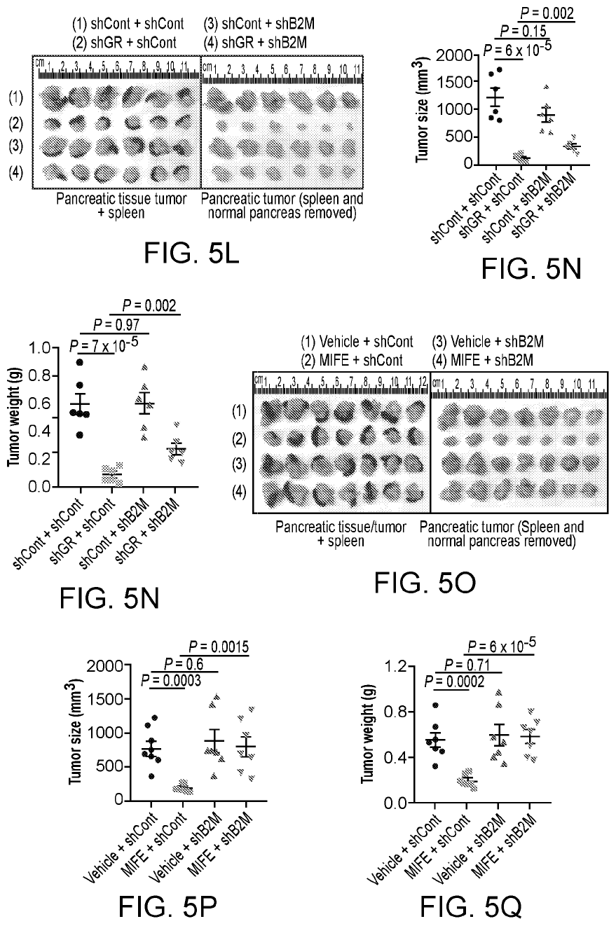

To determine whether GR-mediated regulation of MHC-I is required for the observed anti-tumor effect of GR depletion or inhibition, B2M, an essential structural component of the MHC-I complex, was knocked down in HY24409 cells (FIG. 5A), which abrogated the upregulation of surface MHC-I induced by GR depletion or inhibition (FIGS. 5B-5E). In GR-knockdown or mifepristone-treated HY24409 pancreatic tumors, knockdown of B2M depleted surface MHC-I in vivo (FIGS. 5F-5G), decreased the percentages of tumor-infiltrating CD8+ T cells and granzyme B+ CTLs (FIGS. 5H-5K), and rescued tumor growth (FIGS. 5L-5Q). Taken together with the CD8+ T cell depletion data described above (FIGS. 3B-3E; FIGS. 3G-3J), these results demonstrate that increased surface expression of MHC-I on pancreatic tumor cells upon GR knockdown or inhibition is a prerequisite for increased CD8+ T cell infiltration and tumor suppression.

### Example 3: Tumor Cell-Specific GR Depletion or Pharmacologic GR Inhibition Sensitizes PDAC to Immunotherapy

Pancreatic cancer is highly resistant to ICB therapy. Even targeting multiple immune checkpoints has failed in clinical trials (Leinwand, J. & Miller, G. Regulation and modulation of antitumor immunity in pancreatic cancer. Nat. Immunol. 21, 1152-1159 (2020)). Similarly, female mice with orthotopic implantation of the female KPC line HY 15549 do not respond to ICB by anti-CTLA-4 and anti-PD-1 treatment, even when used in combination. (Yamamoto, K. et al. Autophagy promotes immune evasion of pancreatic cancer by degrading MHC-I. Nature 581, 100-105 (2020)).

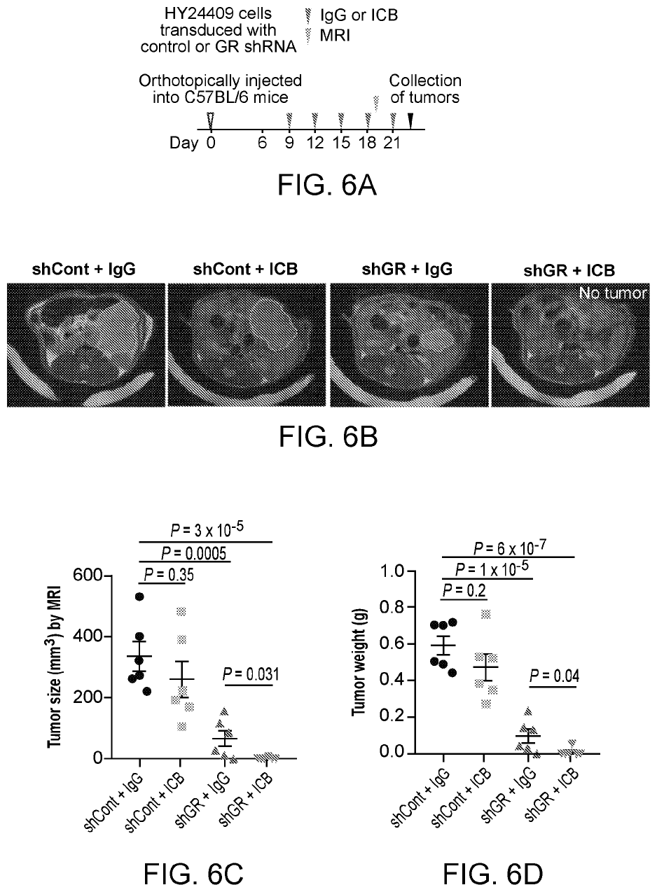

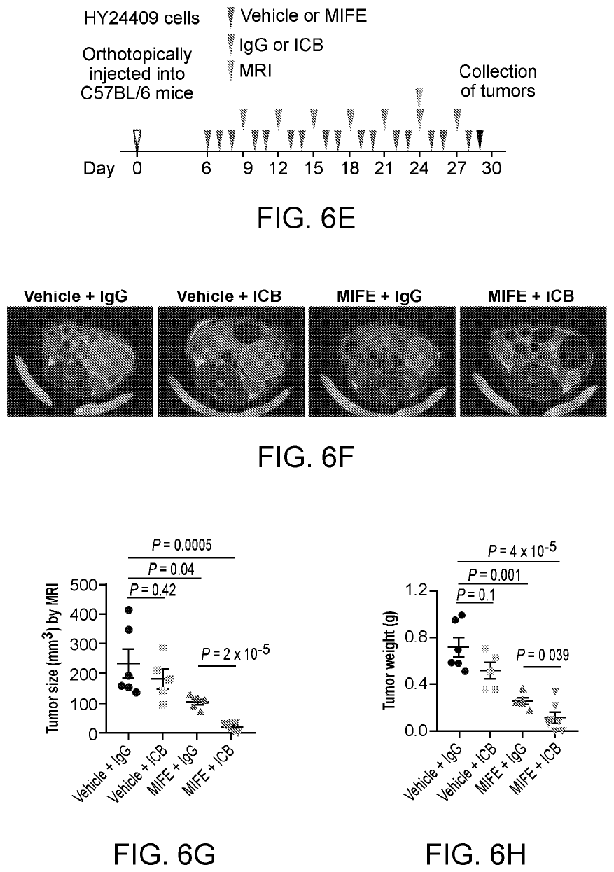

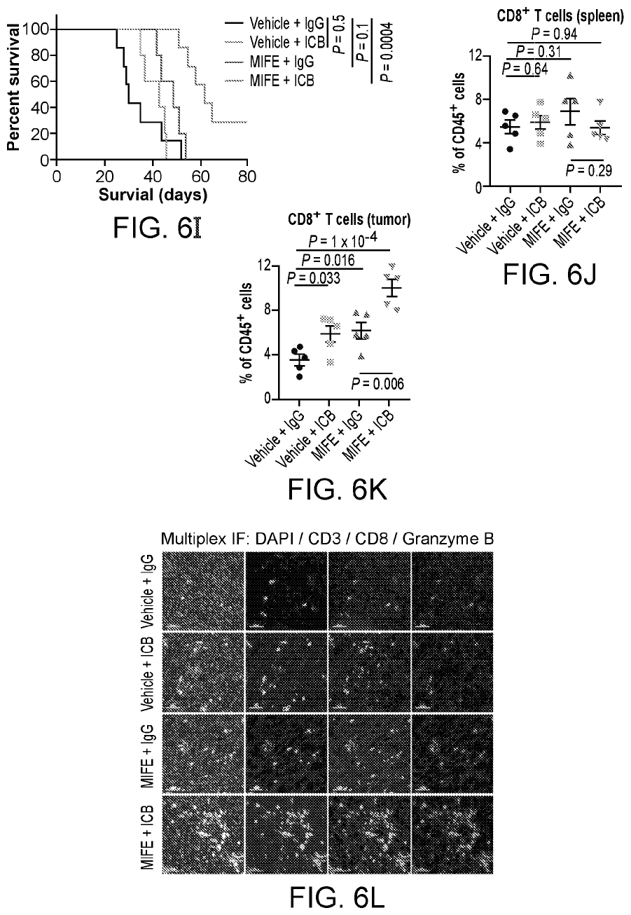

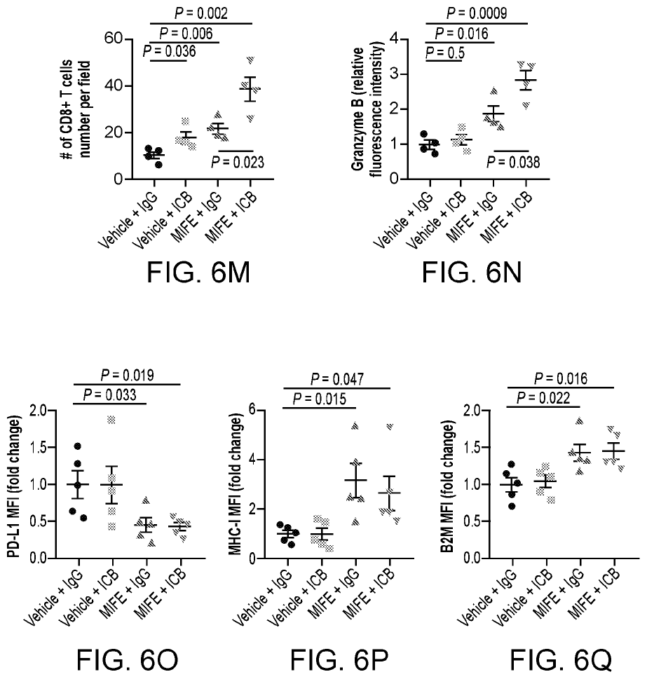

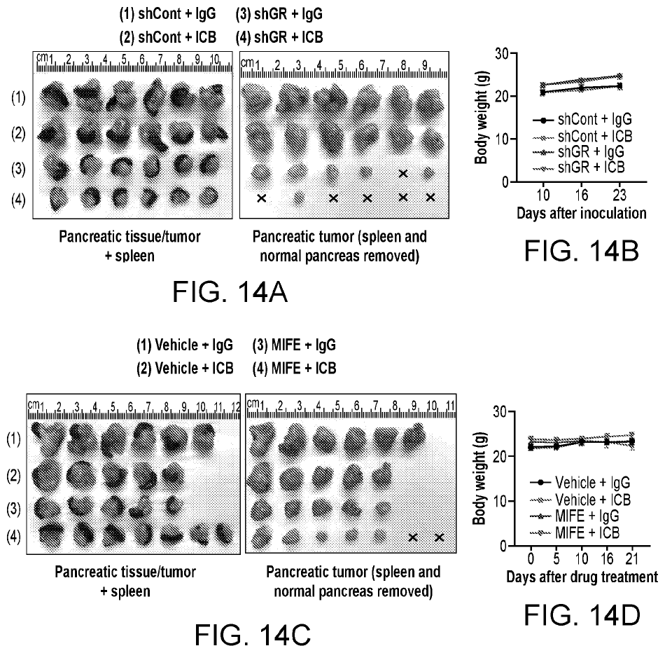

Similarly, male C57BL/6 mice bearing orthotopic pancreatic tumors formed by the male KPC line HY24409 were treated with dual ICB drugs (anti-CTLA-4 and anti-PD-1 antibodies), finding no significant effect on tumor size or weight (FIGS. 6A-6D). Strikingly, shRNA-mediated knockdown of GR in HY24409 cells rendered otherwise ICB-resistant orthotopic pancreatic tumors highly sensitive to dual ICB treatment (FIGS. 6A-6D and FIGS. 14A), without causing body weight loss (FIG. 14B), which underscores a tumor cell-specific role of GR in regulating immunotherapeutic response.

Next, the translatability of the above results was assessed, finding that the combination treatment with mifepristone and dual ICB achieved a greater anti-tumor effect than mifepristone as a single agent (FIGS. 6E-6H and FIGS. 14C-14D).

A potential concern about targeting GR is immune-related adverse events. Notably, compared with the vehicle+IgG control, the combination therapy markedly prolonged survival in mice bearing orthotopic pancreatic tumors (log-rank P=0.0004, hazard ratio=0.19, median survival: 30 days vs. 62 days, FIG. 61); in contrast, either dual ICB therapy (P=0.5) or mifepristone treatment alone (P=0.1) did not lead to a significant improvement in survival (FIG. 61). Thus, treatment with the clinical GR antagonist mifepristone was shown to sensitize ICB-refractory PDAC to anti-CTLA-4 and anti-PD-1 antibodies, resulting in not only substantial tumor growth inhibition but also significant survival benefit.

Similar to the experiments described herein (FIGS. 4G-4I and FIGS. 13I-13K), an increase was reproducibly observed in tumor infiltration by CD8+ T cells and granzyme B+CTLs, but not in splenic CD8+ T cells, upon systemic mifepristone treatment, based on flow cytometric analysis and multiplex immunofluorescent staining (FIGS. 6J-6N). Dual ICB treatment also increased tumor-infiltrating CD8+ T cells, but did not increase the activity of CTLs, as gauged by granzyme B (FIGS. 6K-6N). Notably, the combination treatment with mifepristone and dual ICB further augmented the abundance of tumor-infiltrating CD8+ T cells and granzyme B+ CTLs, compared with mifepristone treatment alone (FIGS. 6K-6N). Relative to the control group, mifepristone-treated pancreatic tumors showed a decrease in cell-surface PD-L1 (FIG. 6O) as well as an increase in cell-surface MHC-I (H-2K^{b}; FIG. 6P) and B2M (FIG. 6Q), either with or without co-treatment with dual ICB.

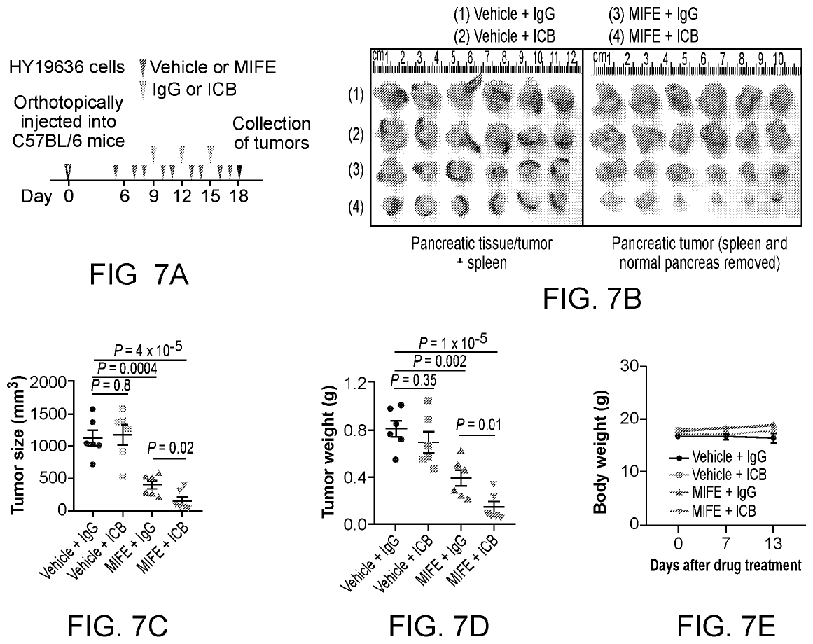

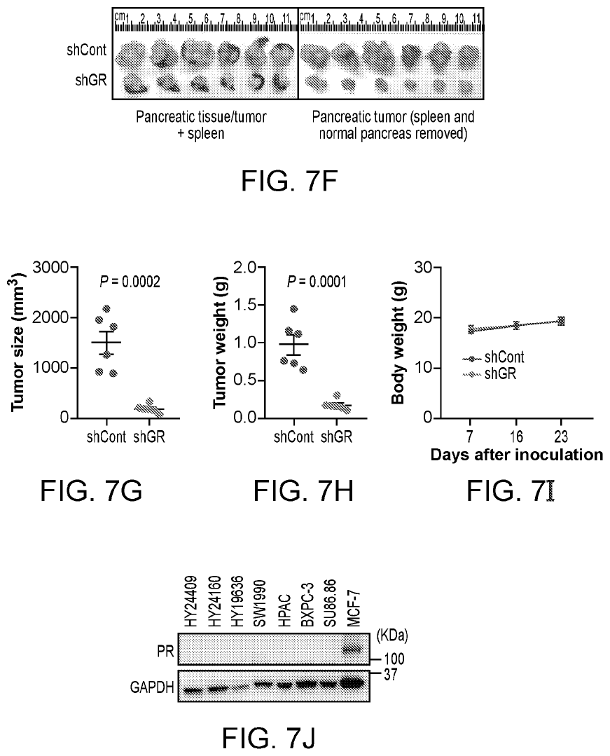

Furthermore, in female C57BL/6 mice bearing orthotopic tumors formed by the female KPC cell line, HY19636, treatment with mifepristone markedly inhibited tumor growth and rendered sensitivity to dual ICB, without reducing body weight (FIGS. 7A-7E). In addition, knockdown of GR in HY19636 cells substantially reduced orthotopic pancreatic tumor size and weight, without altering the body weight of female C57BL/6 hosts (FIGS. 7F-7I), which underscores the relevance of tumor cell-specific GR. The findings hold true in both males and females. Mifepristone is an antagonist of both GR and the progesterone receptor (PR). (Fleseriu, M., et al. Mifepristone, a glucocorticoid receptor antagonist, produces clinical and metabolic benefits in patients with Cushing's syndrome. J Clin Endocrinol Metab 97, 2039-2049 (2012); Spitz, I. M. & Bardin, C. W. Mifepristone (RU 486)—a modulator of progestin and action. N Engl J Med 329, 404-412 (1993)). However, the mouse and human PDAC cell lines used in this study showed no detectable PR expression (FIG. 7J; an ER+PR+breast cancer cell line, MCF-7, was used as a positive control). Thus, the mifepristone effects in this study were attributed to GR.

### Example 4: GR Correlates with PD-L1 Expression, Low MHC-I Expression, and Poor Survival in PDAC

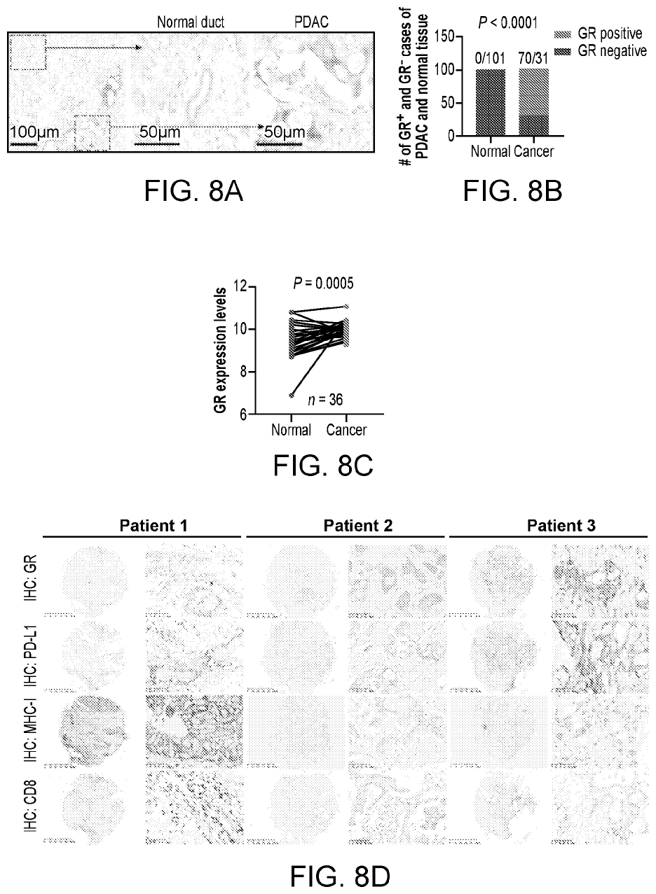

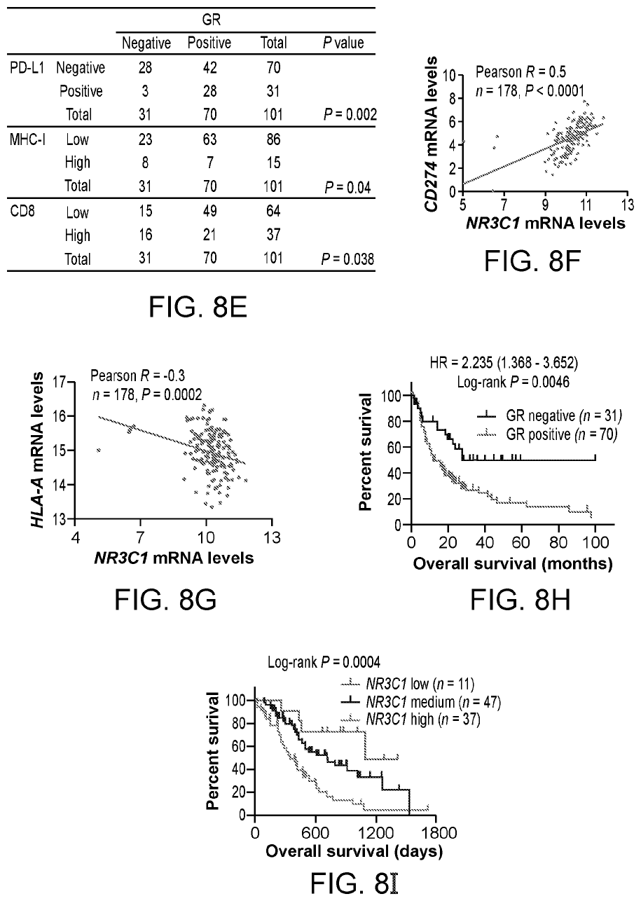

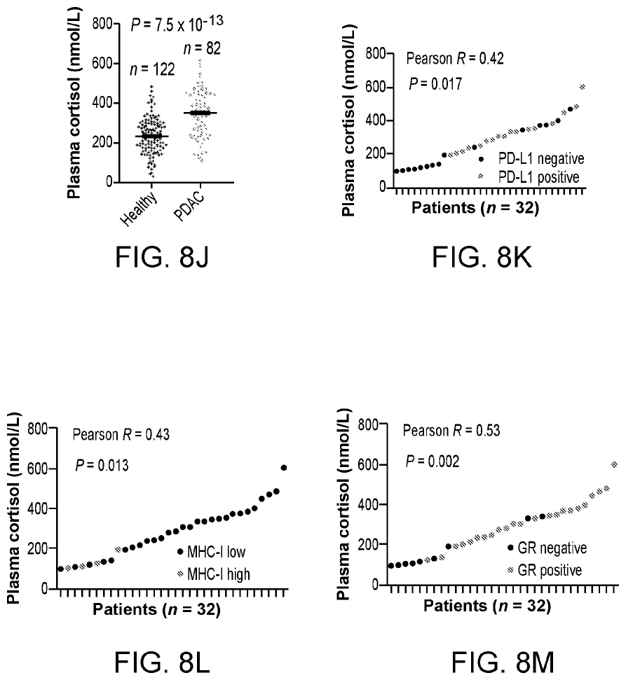

To assess the relevance of GR in human pancreatic cancer, pancreatic tissue microarrays (TMAs) from 101 patients with PDAC were constructed and immunohistochemical staining of GR was performed. Notably, 70 of 101 PDAC tumors showed positive GR staining, whereas none of the adjacent normal pancreatic duct tissues were GR-positive (FIGS. 8A-8B). Consistently, based on the gene expression data from paired samples (study based on methods of GSE15471; Badea, L., et al., Combined gene expression analysis of whole-tissue and microdissected pancreatic ductal adenocarcinoma identifies genes specifically overexpressed in tumor epithelia. Hepatogastroenterology 55, 2016-2027 (2008)), mRNA levels of GR (encoded by NR3 (′1) were upregulated in PDAC relative to paired normal pancreatic tissue (FIG. 8C).

TMAs were immunostained for PD-L1, MHC-I, and CD8, finding that 28 of 31 (90.3%) GR-negative PDAC tumors were also negative for PD-L1, whereas 63 of 70 (90%) GR-positive PDAC tumors had low levels of MHC-I (FIG. 8D-8E). Moreover, tumoral GR protein correlated with low levels of tumor-infiltrating CD8+ cells (FIGS. 8D-8E), without a significant correlation with CD3+ cells (data not shown). Similarly, analysis of the gene expression data from The Cancer Genome Atlas (TCGA) revealed a positive correlation of NR3C1 mRNA levels with CD274 (encoding PD-L1) mRNA levels and a moderate inverse correlation of NR301 mRNA levels with HLA-A mRNA levels in pancreatic cancer (FIGS. 8F-8G). The TMA data analysis indicated that patients with GR-positive PDAC had much shorter overall survival than patients with GR-negative PDAC (FIG. 8H). Similarly, based on analysis of the International Cancer Genome Consortium (ICGC) data, higher expression of NR3C1 correlated with worse survival in pancreatic cancer patients (FIG. 8I). Taken together, these results indicate that GR expression correlates with PD-L1 expression, low MHC-I expression, low CD8+ T cell infiltration, and poor survival in patients with pancreatic cancer. Plasma levels of cortisol, the natural agonist of GR in humans, were significantly elevated in patients with PDAC compared with healthy volunteers (FIG. 8J). In PDAC patients, plasma cortisol levels positively correlated with tumoral PD-L1 protein expression and inversely correlated with tumoral MHC-I protein expression (FIGS. 8K-L). A significant positive correlation between plasma cortisol levels and GR proteins levels was observed in pancreatic tumors (FIG. 8M). Collectively, these data support a link between activation of GR signaling and pancreatic cancer immune evasion.

### Example 5: Methods

### Mouse Experiments

All animal studies were performed in accordance with a protocol (PI: Li Ma) approved by the Institutional Animal Care and Use Committee (IACUC) of MD Anderson Cancer Center. Animals were housed at 70° F.-74° F. (set point: 72° F.) with 40%-55% humidity (set point: 45%). The light cycle of animal rooms is 12 hours of light and 12 hours of dark. Orthotopic injection of PDAC cells was performed as described in Yamamoto, K. et al. Autophagy promotes immune evasion of pancreatic cancer by degrading MHC-I. Nature 581, 100-105 (2020). Approximately 4-8×10⁴ HY24409 (male), HY24160 (male), or HY19636 (female) cells, with >95% viability in trypan blue exclusion assays, were suspended in a mixture of 10 μl phosphate-buffered saline (PBS) and 10 μl Matrigel (VWR, 47743-720) and were then injected into a region of the pancreas just beneath the spleen. Drug treatment was started after confirming tumor formation by magnetic resonance imaging (MRI), and mice were randomly assigned to different treatment groups. To assess the importance of immune regulation, male NSG (non-obese diabetic; severe combined immunodeficiency; interleukin-2 receptor gamma chain null) mice were used. Six-week-old NSG mice received subcutaneous injection of approximately 4-10×10⁴ HY24409 or HY24160 cells.

For in vivo CD8+ T cell depletion, mice received intraperitoneal injection of anti-mouse CD8a antibody (200 μg, Bio X Cell, BE0061, clone 2.43; RRID: AB_1125541) or rat IgG2b isotype control (200 μg, Bio X Cell, BE0090, clone LTF-2; RRID: AB_1107780) at the indicated times. Depletion was confirmed by flow cytometric analysis of dissociated tumor samples or blood samples with antibodies targeting non-competing CD8 epitopes. For immune checkpoint blockade experiments, mice received intraperitoneal injection of anti-mouse PD-1 antibody (100 μg, Bio X Cell, BE0146, clone RMP1-14; RRID: AB_10949053) and anti-mouse CTLA-4 antibody (100 μg, Bio X Cell, BE0032, clone UC10-4F10-11; RRID: AB_1107598), or rat IgG2a isotype control (100 μg, Bio X Cell, BE0089, clone 2A3; RRID: AB_1107769) and control hamster IgG (100 μg, Bio X Cell, BE0091; RRID: AB_1107773) at the indicated times. For GR antagonist treatment, mifepristone (Selleckchem, S2606) was dissolved in vehicle solvent containing 5% dimethylacetamide (Sigma-Aldrich, D137510) and 95% olive oil (Sigma-Aldrich, 01514). The dose was 60 mg kg-1 by oral gavage, which was calculated based on a phase 2 clinical trial (ClinicalTrials.gov, identifier: NCT02642939) and previously described dose conversion between animals and humans. Mifepristone was administered on a schedule of twice every 3 days.

Orthotopic pancreatic tumor size was determined by MRI. Subcutaneous tumor size was measured with a caliper. No tumors exceeded the IACUC-defined maximum diameter of 2 cm. Animal body weight was measured throughout the study. Tumor weight was measured at the study endpoint after the mice were euthanized.

### Multiplex Immunofluorescent Staining of Mouse Tumors

After euthanasia, mouse tissues were fixed in 10% neutral-buffered formalin (Sigma-Aldrich, HT501128) overnight, washed with PBS, transferred to 70% ethanol, embedded in paraffin, and sectioned (5 μm thick). The Opal Polaris 7 Color Detection Kit (Akoya Biosciences, NEL861001KT) was used for multiplex immunofluorescent staining according to the manufacturer's protocol. In brief, formalin-fixed paraffin-embedded (FFPE) slides were baked at 65° C. for 1 hour, deparaffinized in xylene (3×10 min), rehydrated through degraded alcohols (100% ethanol, 3×5 min; 95% ethanol, 1×5 min; 75% ethanol, 1×5 min; and 50% ethanol, 1×5 min), briefly rinsed in distilled water, and fixed in 10% formalin for 20 min. After fixation, slides were briefly rinsed in water and placed in AR6 buffer (AR6001KT, provided in the Opal Polaris 7 Color Detection Kit). Heat-induced epitope retrieval was done with a 2100-Retriever, after which the slides were cooled down at room temperature for 30-60 min, rinsed in distilled water, followed by Tris-buffered saline with 0.05% Tween-20 (TBST). A hydrophobic barrier pen (Vector Laboratories, H-4000-2) was used to create a hydrophobic barrier around the tissue. Slides were then placed in a humidified chamber with blocking buffer (ARD10011EA, provided in the Opal Polaris 7 Color Detection Kit) at room temperature for 10 min. Next, slides were incubated with the primary antibody at room temperature for 1-2 hours or at 4° C. overnight. Slides were washed in TBST (3×2 min) with agitation and then placed in AR6 buffer. Heat-induced epitope retrieval was performed again, and the protocol was repeated until all targets were detected. Slides were mounted with mounting medium with DAPI (Vector Laboratories, H-1200) and sealed with coverslips. The primary antibodies are as follows: anti-CD3 (1:200, Cell Signaling Technology, 99940S, RRID: AB_2755035) used with Opal480 (1:100), anti-CD8 (1:200, Cell Signaling Technology, 98941S, RRID: AB_2756376) used with Opal520 (1:100), and anti-granzyme B (1:200, Cell Signaling Technology, 44153S, RRID: AB_2857976) used with Opal690 (1:100). The finished slides were scanned by the Vectra Polaris Automated Quantitative Pathology Imaging System. The number of positive cells and the fluorescence intensity were quantitated by Image J software. CyTOFCyTOF experiments were performed as described in Zhao, D., et al. Chromatin Regulator CHD1 Remodels the Immunosuppressive Tumor Microenvironment in PTEN-Deficient Prostate Cancer. Cancer Discov 10, 1374-1387 (2020). Briefly, tumor samples were dissociated on the gentleMACS Dissociator (Miltenyi Biotec) with the Mouse Tumor Dissociation Kit (Miltenyi Biotec, 130-096-730) and were depleted of red blood cells using RBC Lysis Buffer (BioLegend, 420301). 1×10⁶ cells per sample were used for staining. For dead cell staining, cells were incubated with cisplatin (2.5 μM, Fisher Scientific, NC0637801) for 1 min. Cells were Fcblocked with an anti-CD16/CD32 antibody (BioLegend, 101320) for 10-20 min and then incubated with the CyTOF surface antibody mix for 30-60 min. Cells were incubated with 1.6% paraformaldehyde (PFA) in PBS for 30 min and incubated in cold 100% methanol at −20° C. overnight. For intracellular staining, cells were incubated with the CyTOF intracellular antibody mix for 30-60 min. For singlet discrimination, cells were washed and incubated with Cell-ID Intercalator-Ir (Fluidigm, 201192A) at 4° C. overnight. CyTOF data were analyzed by FlowJo and Cytobank. Antibodies used for CyTOF are listed in Tables 1 and 2. Gating methods used for CyTOF analysis are listed in Table 3.

TABLE 1
Antibodies used for CyTOF analysis for HY24409 tumors:

| | | Intracellular | | | |
|---|---|---|---|---|---|
| Target | Label | Staining | Clone# | Source | Catalog# |
| CD11b | 139La | FALSE | M1/70 | BioLegend | 101249 |
| CD11c | 142Nd | FALSE | N418 | BioLegend | 117302 |
| CD19 | 149Sm | FALSE | 4D5 | BioLegend | 115502 |
| CD25 | 150Nd | FALSE | 3C7 | BioLegend | 101902 |
| CD3, CD3e | 152Sm | FALSE | 145-2C11 | BioLegend | 100302 |
| CD4(Ms) | 115In | FALSE | RM4-5 | BioLegend | 100506 |
| CD45(Ms) | 89Y | FALSE | 30-F11 | DVS- | 3089005B |
| | | | | Fluidigm | |
| CD8a | 146Nd | FALSE | 53-6.7 | BioLegend | 100702 |
| F4/80 | 171Yb | FALSE | D2S9R | CST | 70076BF |
| Foxp3 | 158Gd | TRUE | FJK-16s | DVS- | 3158003A |
| | | | | Fluidigm | |
| I-A/I-E, MHC-II | 209Bi | FALSE | M5/114.15.2 | DVS- | 3209006B |
| | | | | Fluidigm | |
| Ly-6G/Ly-6C, Gr- | 141Pr | FALSE | RB6-8C5 | BioLegend | 108402 |
| 1 | | | | | |
| NK1.1 | 170Er | FALSE | PK136 | BioLegend | 108702 |

TABLE 2
Antibodies used for CyTOF analysis for HY24160 tumors:

| | | Intracellular | | | |
|---|---|---|---|---|---|
| Target | Label | Staining | Clone | Source | Catalog# |
| CD45(Ms) | 89Y | FALSE | 30-F11 | DVS- | 3089005B |
| | | | | Fluidigm | |
| CD3, CD3e | 152Sm | FALSE | 145-2C11 | BioLegend | 100302 |
| CD8a | 146Nd | FALSE | 53-6.7 | BioLegend | 100702 |
| CD4(Ms) | 115In | FALSE | RM4-5 | BioLegend | 100506 |
| F4/80 | 173Yb | FALSE | BM8 | BioLegend | 123102 |
| CD11b | 139La | FALSE | M1/70 | BioLegend | 101249 |
| Ly-6C | 162Dy | FALSE | HK1.4 | DVS- | 3162014B |
| | | | | Fluidigm | |
| Ly-6G | 141Pr | FALSE | 1A8 | DVS- | 3141008B |
| | | | | Fluidigm | |
| CD25 | 150Nd | FALSE | 3C7 | BioLegend | 101902 |
| Foxp3 | 158Gd | TRUE | FJK-16s | DVS- | 3158003A |
| | | | | Fluidigm | |
| CD19 | 149Sm | FALSE | 4D5 | BioLegend | 115502 |
| CD11c | 142Nd | FALSE | N418 | BioLegend | 117302 |
| NK1.1 | 170Er | FALSE | PK136 | BioLegend | 108702 |
| I-A/I-E, MHC-II | 209Bi | FALSE | M5/114.15.2 | DVS- | 3209006B |
| | | | | Fluidigm | |

TABLE 3
Gating methods used for CyTOF analysis

| | |
|---|---|
| CD8⁺ T cells | CD45⁺CD11b⁻CD3⁺CD8⁺CD4⁻ |
| CD4⁺ T cells | CD45⁺CD11b⁻CD3⁺CD8⁻CD4⁺ |
| Treg cells | CD45⁺CD11b⁻CD3⁺CD8⁻ |
| | CD4⁺Foxp3⁺ |
| Gr-MDSCs | CD45⁺CD11b⁺CD3⁻Gr1⁺ |
| Macrophages | CD45⁺CD11b⁺CD3⁻Gr1⁻ |
| | F4/80⁺ |
| B cells | CD45⁺CD11b⁺CD3⁻CD19⁺ |
| Dendritic cells | CD45⁺CD11b⁺CD3⁻MHC− |
| | II⁺CD11c⁺ |
| NK cells | CD45⁺CD11b⁺CD3⁻NK1.1⁺ |

### Cell Culture

The male PDAC cell lines HY24409 and HY24160 and the female PDAC cell line HY19636 were established from KPC mice (p48-Cre; Kras^{LSL-G12D/+}; Trp53^{loxP/+}) that were backcrossed to a C57BL/6 background (purity: ˜ 98%). (See Bardeesy, N., et al. supra). The human PDAC cell lines SU86.86 (female), SW1990 (male), BXPC-3 (female), Miapaca-2 (male), Capan-1 (male), ASPC-1 (female), Hs766T (male), Colo357/FG (female), L3.6PL (female), Panc28 (female), Capan-2 (male), CF-Pac-1 (male), Panc3 (sex unknown), Panc1 (male), Panc48 (sex unknown), and HPAC (female) were from Mien-Chie Hung's lab stock. The HY24409, HY24160, HY19636, SU86.86, SW1990, and Hs766T cell lines were cultured in RPMI 1640 (Corning, 10-041-CV) supplemented with 10% fetal bovine serum (FBS) (Gibco, 10438-034) and 1% penicillin/streptomycin (Sigma, P4333). The BXPC-3, Miapaca-2, Capan-1, ASPC-1, Colo357/FG, L3.6PL, Panc28, Capan-2, CF-Pac-1, Panc3, Panc1, Panc48, and HPAC cell lines were cultured in DMEM/F-12 (Corning, 10-092-CV) supplemented with 10% FBS and 1% penicillin/streptomycin. Cells were grown in a humidified incubator with 5% CO2 at 37° C., and low-passage stocks were maintained in a centralized lab cell bank. Short tandem repeat profiling and mycoplasma tests were done by the Cytogenetics and Cell Authentication Core at MD Anderson Cancer Center.

For GR activation experiments, cells were cultured in phenol red-free RPMI 1640 (Gibco, 11835-030) supplemented with 5% charcoal-stripped FBS (Gibco, A33821-01) for 3 days, and then were cultured in the presence of dexamethasone (100 nM, Sigma, D2915) with or without mifepristone (100 nM, R&D, 1479/100) for 3-8 hours.

Lentiviral shRNA Infection

For human GR knockdown, two shRNAs, TRCN0000245004 (shGR-1; SED ID NO: 9) and TRCN0000245006 (shGR-2; SEQ ID NO: 10) were used in the pLKO-puro vector from Sigma. Mouse GR knockdown was done with two shRNAs, TRCN0000026186 (SEQ ID NO: 11) and TRCN0000238464 (SEQ ID NO: 12), and mouse B2M knockdown was done with the shRNA TRCN0000295705 (SEQ ID NO: 13), all in the pLKO-puro vector from Sigma. A non-targeting shRNA in the pLKO-puro backbone was used as the control. Lentivirus was produced by transfecting 4 μg lentiviral shRNA plasmid, 3 μg viral packaging plasmid pPAX2, and 1 μg envelope plasmid pMD2.G into HEK293T cells. Cells were infected in the presence of polybrene (5 μg ml⁻¹, Sigma, TR-1003-G), and at 48-72 hours after infection, cells were selected with 2 μg ml⁻¹ puromycin (ThermoScientific, A1113803).

### Flow Cytometry

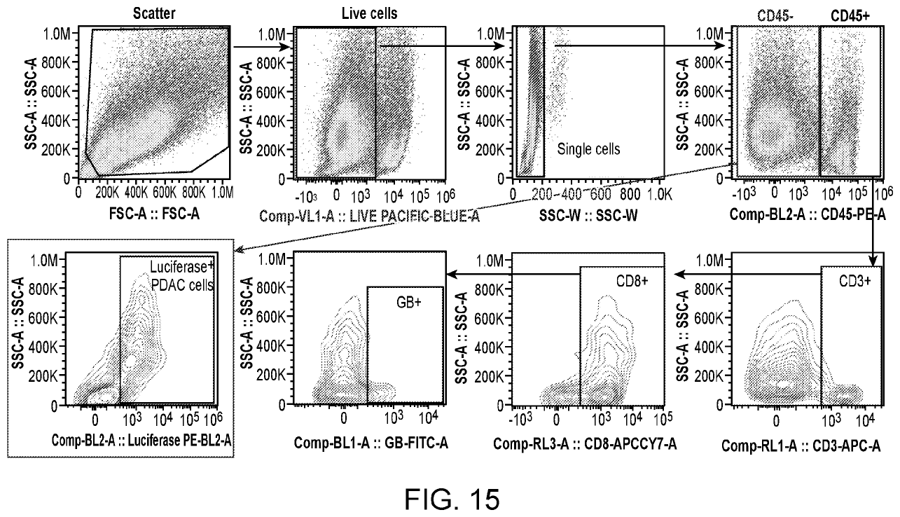

For cultured cell lines, cells were incubated with the Accutase Cell Detachment Solution (BioLegend, 423201) and were washed twice with PBS. For immunophenotyping of tumors, tumor samples were dissociated, depleted of red blood cells, and Fc-blocked as described above. One million cells were stained with the Zombie Aqua Fixable Viability Kit (BioLegend, 423114). Cells were incubated with the indicated antibody diluted in staining buffer (2% FBS in PBS) at 4° C. in the dark for 30-60 min. Then, cells were washed twice and analyzed or further fixed in 1.6% PFA in PBS for 20 min. Intracellular staining was done with the Intracellular Staining Permeabilization Wash Buffer (BioLegend, 421002). Detection of cytokine production ex-vivo was performed as described in Acharya, N., et al. Endogenous Glucocorticoid Signaling Regulates CD8 (+) T Cell Differentiation and Development of Dysfunction in the Tumor Microenvironment. Immunity 53, 658-671 e656 (2020); He, Y., et al. Gut microbial metabolites facilitate anticancer therapy efficacy by modulating cytotoxic CD8 (+) T cell immunity. Cell Metab 33, 988-1000 e1007 (2021); and Xu, S., et al. Uptake of oxidized lipids by the scavenger receptor CD36 promotes lipid peroxidation and dysfunction in CD8 (+) T cells in tumors. Immunity 54, 1561-1577 e1567 (2021). Tumor-infiltrating lymphocytes were enriched by Percoll (Fisher Scientific, 45-001-754) gradient centrifugation. Cells were resuspended in RPMI 1640 containing 10% FBS, stimulated by 50 ng ml⁻¹ PMA (Sigma-Aldrich, P8139-1 MG) and 3 μM ionomycin (R&D Systems, 1704/1) in the presence of 2.5 mg ml⁻¹ Brefeldin A (BioLegend, 420601) at 37° C. for 4 hours. Cells were processed for surface marker staining as described above. For intracellular cytokine staining, cells were fixed in Fixation Buffer (BioLegend, 420801) for 20 min and were washed two times with Permeabilization buffer (BioLegend, 421002). Cells were then stained with intracellular antibodies for 30 min. After staining, cells were analyzed on an Invitrogen Attune NxT Acoustic Focusing Cytometer and analyzed by FlowJo software. Tumor cells were gated by ZombieDye⁻CD45⁻luciferase⁺, CTLs by ZombieDye⁻CD45⁺CD3⁺CD8⁺, and granzyme B-positive CTLs by ZombieDye⁻CD45⁺CD3⁺CD8⁺GB⁺. Gating strategies are shown in FIG. 15. Antibodies used for flow cytometry are listed in Table 4.

TABLE 4
Antibodies for flow cytometry

| | | Intracellular | | | | |
|---|---|---|---|---|---|---|
| Target | Color | Staining | Clone# | Specificity | Source | Catalog# |
| PD-L1 | APC | FALSE | 10F.9G2 | Mouse | BioLegend | 124312 |
| CD45 | PE | FALSE | 30-F11 | Mouse | BioLegend | 103106 |
| CD45 | FITC | FALSE | 30-F11 | Mouse | BioLegend | 103108 |
| CD3 | APC | FALSE | 145-2C11 | Mouse | BioLegend | 100312 |
| CD8 | APC- | FALSE | 53-6.7 | Mouse | BioLegend | 100714 |
| | Cy7 | | | | | |
| CD4 | FITC | FALSE | GK1.5 | Mouse | BioLegend | 100406 |
| B2M | APC | FALSE | A16041A | Mouse | BioLegend | 154506 |
| B2M | PE | FALSE | A16041A | Mouse | BioLegend | 156504 |
| H-2K^{b} | PE-Cy7 | FALSE | AF6-88.5 | Mouse | BioLegend | 116520 |
| H-2K^{b} | APC | FALSE | AF6-88.5 | Mouse | BioLegend | 116518 |
| H-2K^{b}/D^{b} | — | FALSE | 28-8-6 | Mouse | BioLegend | 114602 |
| Luciferase | PE | TRUE | Luci 21 | — | Novus | NB600- |
| | | | 1-107 | | Biologicals | 307PE |
| HLA- | PE | FALSE | W6/32 | Human | BioLegend | 311406 |
| A/B/C | | | | | | |
| HLA- | PE-Cy5 | FALSE | G46-2.6 | Human | BD | 555554 |
| A/B/C | | | | | | |
| B2M | FITC | FALSE | 2M2 | Human | BioLegend | 316304 |
| PD-L1 | APC | FALSE | 29E.2A3 | Human | BioLegend | 329708 |
| IL-2 | PE | TRUE | JES6-5H4 | Mouse | BioLegend | 503807 |
| TNFα | PE | TRUE | MP6- | Mouse | BioLegend | 506305 |
| | | | XT22 | | | |
| IFNγ | PE-Cy7 | TRUE | XMG1.2 | Mouse | BioLegend | 505825 |
| PD-1 | PE-Cy7 | FALSE | 29F.1A12 | Mouse | BioLegend | 135215 |
| LAG-3 | PE | FALSE | C9B7W | Mouse | BioLegend | 125207 |
| Tim-3 | PE | FALSE | RMT3-23 | Mouse | BioLegend | 119703 |

### Luciferase Reporter Assay

Human PD-L1 promoter-reporter constructs (Coelho, M. A., et al. Oncogenic RAS Signaling Promotes Tumo Immunoresistance by Stabilizing PD-L1 mRNA. Immunity 47, 1083-1099 e1086 (2017)) were purchased from Addgene (Addgene numbers: 107002, 107003, 107004, 107006, and 107007). The GR activity reporter plasmid was purchased from Qiagen (ID: C82DB0D7-3D10-4B1C-A358-411558D2DE01). The promoter regions of human HLA-B, HLA-C, and B2M were PCR-amplified from genomic DNA of the SU86.86 cell line (PCR primers are listed as SEQ ID NOs.: 1-6. The linearized pGL3-basic plasmid was amplified by PCR (Forward primer SEQ ID NO: 7; reverse primer SEQ ID NO: 8), and PCR products of promoter regions were ligated to linearized pGL3-basic using the In-Fusion HD Cloning Kit (Takara Bio, 638909).

The firefly luciferase reporter containing the human gene promoter was co-transfected with a Renilla luciferase vector (for normalization) into SU86.86 cells using jetPRIME transfection reagent (VWR, 89129-922). One day after transfection, cells were treated with IFNγ (10 ng ml⁻¹, 8 h) or dexamethasone (100 nM, 8 h). Firefly and Renilla luciferase activities were measured using a Dual-Luciferase Reporter Assay (Promega, E1910) on a microplate reader according to the manufacturer's protocol. Firefly luciferase activity was normalized to Renilla luciferase activity.

### Quantitative Reverse-Transcription PCR

Total RNA was extracted using TRIzol Reagent (Life technologies, 15596018) and the PuroLink RNA Mini Kit (Invitrogen, 12183018A), and then reversed-transcribed with the iScript Reverse Transcription Supermix (Bio-Rad, 1708841). Quantitative PCR was performed with the iTaq Universal SYBR Green Supermix (Bio-Rad, 1725124) on a CFX96 real-time PCR machine (Bio-Rad). The mRNA level was calculated using the ΔCt method and normalized by GAPDH. Sequences for qPCR primers are listed in Table 5.

### Immunoblotting

Cultured cells or homogenized mouse tissues were lysed in RIPA lysis buffer (Sigma-Aldrich, 20-188) containing protease inhibitors (GenDEPOT, P3100-001) and phosphatase inhibitors (GenDEPOT, P3200-001). Proteins were resolved on 4%-12% (GenScript, M00654) or 4%-20% (GenScript, M00657) precast gradient gels and transferred to a nitrocellulose membrane using the Trans-Blot Turbo Transfer System (Bio-Rad, 1704150). Membranes were blocked with 10% non-fat milk in TBST and incubated with the primary antibody at 4° C. overnight, followed by incubation with the secondary antibody conjugated with horseradish peroxidase (HRP). The bands were visualized with enhanced chemiluminescence substrate (ThermoFisher Scientific, 34578). Primary antibodies used are as follows: antibodies against GR (1:1,000, Proteintech, 24050-1-AP, RRID: AB_2813890), phospho-GR (Ser211) (1:1,000, Cell Signaling Technology, 4161S, RRID: AB_2155797), GAPDH (1:2,000, Proteintech, 60004-1-IG, RRID: AB_2107436), PD-L1 (1:1,000, Cell Signaling Technology, 13684S, RRID: AB_2687655), MHC-I (1:500, Santa Cruz Biotechnology, sc-55582, RRID: AB_831547), MHC-I (1:500, Santa Cruz Biotechnology, sc-32235, RRID: AB_627934), B2M (1:1,000, Cell Signaling Technology, 12851S, RRID: AB_2716551), phospho-STAT1 (1:1,000, Cell Signaling Technology, 9167S, RRID: AB_561284), STAT1 (1:1,000, Cell Signaling Technology, 14994S, RRID: AB_2737027), and PR (1:1,000, Proteintech, 25871-1-AP, RRID: AB_2880277).

### Human Samples and Plasma Cortisol Measurement

The human tissue microarray and plasma samples were from the Cancer Hospital of the University of Chinese Academy of Sciences, Zhejiang Cancer Hospital (Hangzhou, China). All tissue and blood samples were collected with informed consent. The collection and use of human samples were approved by the Ethics Committee of Cancer Hospital of the University of Chinese Academy of Sciences, following the Declaration of Helsinki ethical guidelines. Human plasma cortisol levels were quantitated with an ELISA kit from ENZO Life Sciences (ADI-900-071) using blood samples collected at the same time (9 am local time).

### Immunohistochemical Staining (IHC) of Tissue Microarrays

Immunohistochemical staining of tissue microarrays was done on the FFPE slides. After the xylene-alcohol-water workflow and the antigen retrieval step as described above, slides were incubated with blocking solution (Vector Laboratories, SP-6000-100) at room temperature for 10 min, followed by a quick wash with PBS. Slides were then incubated with 20% horse serum (Vector Laboratories, PK-7200) at room temperature for 20 min, followed by incubation with the primary antibody at 4° C. overnight. Slides were quickly rinsed with PBS and incubated with biotinylated universal secondary antibody (Vector Laboratories, PK-7200) or goat IgG HRP-conjugated antibody (R&D systems, HAF017) at room temperature for 30 min, followed by a quick rinse with PBS and incubation with ABC reagent (Vector Laboratories, SK-4100) for 30 min. DAB solution (Vector Laboratories, SK-4100) was applied at room temperature for 45 seconds, followed by counterstaining with hematoxylin QS (Vector 24 Laboratories, H-3404) at room temperature for 1 min, mounting (using medium: Vector Laboratories, H-5000-60), and sealing. Primary antibodies used for IHC are antibodies against GR (1:200, Sigma-Aldrich, SAB4501309; RRID: AB_10744954), PD-L1 (1:200, GeneTex, GTX01796), MHC-I (1:200, Santa Cruz Biotechnology, sc55582; RRID: AB_831547), CD8 (1:100, MXB Biotechnologies, RMA-0514), and CD3 (1:150, ZSGB-BIO, ZM-0417). In PDAC cells, PD-L1 and MHC-I were predominantly localized on the cell membrane, whereas GR was present in both the nucleus and the cytoplasm. Slides were scanned using a fully automated digital pathology slide system (KFBIO, KF-PRO-005). Histopathological review and IHC scoring were done by two pathologists (Wenjuan Yin and Weiya Xia). Positive and negative scores were assigned to GR and PD-L1. High and low scores were assigned to MHC-I, and expression was deemed high if >20% of tumor cells were MHC-I positive. High and low scores were assigned to CD8 and CD3, and expression was deemed high if >10 cells per high-power field (×400) were positive.

### Chromatin Immunoprecipitation (ChIP)

SU86.86 cells were grown to 40% confluence in 15-cm dishes in RPMI with 10% FBS, followed by 72-hour incubation in phenol red-free RPMI supplemented with 5% charcoal-stripped FBS. Cells were treated with vehicle, 100 nM dexamethasone, or 100 nM dexamethasone+mifepristone for 30 minutes. The Simplechip® Plus Enzymatic Chromatin IP Kit (Magnetic Beads) (Cell Signaling Technology, 9005S) was used for the following steps. Cells were cross-linked with 1% formaldehyde, quenched with glycine, and harvested. After cell lysis with ChIP lysis buffer, cells were digested with Micrococcal Nuclease and sonicated to achieve the majority of DNA fragments between 200 bp and 500 bp. GR was immunoprecipitated using 4 μg ChIP-grade rabbit anti-GR antibody (Proteintech, 24050-1-AP, RRID: AB_2813890), and 4 μg of rabbit IgG (Cell Signaling Technology, 2729) was used as a control. Chromatin was eluted from GR ChIP following the manufacturer's protocol. GR-binding sites were predicted by PROMO (http://alggen.lsi.upc.es/cgi-bin/promo_v3/promo/promoinit.cgi?dirDB=TF_8.3; last accessed Nov. 26, 2021). Primers for ChIP-qPCR are listed in Table 5.

### Chemokine Array

Chemokine array was performed with conditioned medium from HY24409 cells treated with vehicle or mifepristone (20 μM, 48 hours) using the Proteome Profiler Mouse Chemokine Array Kit (R&D system, ARY020) following the manufacturer's instructions.

Cell cycle analysis Cells were seeded in 6-well plates (2×10⁵ cells per well) and treated with 2 mM thymidine (Sigma-Aldrich, T9250-1G) for 18 hours, fresh medium for 9 hours, and 2 mM thymidine again for 17 hours to arrest cells in G1/S phases for synchronization. Released cells were collected at the indicated time points by centrifugation, washed in cold PBS, resuspended in 1 ml of 70% ethanol, and stored at −20° C. overnight. Cells were then collected by centrifugation and washed two times with cold PBS. To ensure that only DNA was stained, cells were treated with 50 μl of 100 μg ml⁻¹ RNase (New England Biolabs, T3018L), and added 425 μl of cell staining buffer (2% FBS in PBS) and 25 μl of propidium iodide solution (BioLegend, 421301). After staining, samples were analyzed by flow cytometry. Cells were gated for PI staining and the cells in G1, S, and G2/M phases were quantitated using FlowJo software.

### Computational Data Analysis

GR (encoded by NR3C1) mRNA levels in paired normal pancreatic tissue and PDAC were obtained from the dataset GSE15471 in the Gene Expression Omnibus (https://www.ncbi.nlm.nih.gov/geo/; last accessed Nov. 26, 2021). TCGA gene expression data were obtained from The Cancer Genome Atlas data portal (https://tcga-data.nci.nih.gov/tcga/dataAccessMatrix.htm; last accessed Nov. 26, 2021). The correlation of expression levels of two genes was analyzed using the R corrplot package and the cor function. ICGC gene expression and clinical data were obtained from the International Cancer Genome Consortium data portal (https://dcc.icgc.org/repositories; last accessed Nov. 26, 2021). Survival analysis was performed using the R survival package. Patient stratification was done using the R kmeans function on gene expression values.

### Statistics and Reproducibility

Except for the animal studies, each experiment was repeated at least three times. For qPCR assays of cell lines, n=3 technical replicates were used per sample, and a representative set from three independent experiments is shown. For all other experiments (including qPCR assays of mouse tissues and ChIP-qPCR assays), biological replicates were used. Unless otherwise noted, data are presented as mean±s.e.m, and Student's 1-test (two-tailed) was used to compare two groups of independent samples. The data analyzed by the t-test were normally distributed; an F-test was used to compare variances, and the variances were not significantly different. Therefore, when using a t-test, equal variance was assumed, and no data points were excluded from the analysis. P<0.05 was considered statistically significant.

TABLE 5
Sequences

| | |
|---|---|
| SEQ ID NO: 1 | TTTCTCTATCGATAGGTCATGTTTGTAGACTT |
| B2M Forward | CCCAAATTTGCC |
| Primer for luciferase | |
| reporter plasmid | |
| construction (5′ to 3′) | |
| (nucleic acid sequence) | |
| SEQ ID NO: 2 | CCGGAATGCCAAGCTAACTTGGAGAAGGGA |
| B2M Reverse | AGTCACGG |
| Primer for luciferase | |
| reporter plasmid | |
| construction (3′ to 5′) | |
| (nucleic acid sequence) | |
| SEQ ID NO: 3 | TTTCTCTATCGATAGGCGCAGCTAAAACTGG |
| HLA-B Forward | TGAAT |
| Primer for luciferase | |
| reporter plasmid | |
| construction (5′ to 3′) | |
| (nucleic acid sequence) | |
| SEQ ID NO: 4 | CCGGAATGCCAAGCTGCGTCTGAGGAGACTC |
| HLA-B Reverse | TGAGT |
| Primer for luciferase | |
| reporter plasmid | |
| construction (3′ to 5′) | |
| (nucleic acid sequence) | |
| SEQ ID NO: 5 | TTTCTCTATCGATAGAAACCCATAAAATTCAT |
| HLA-C Forward | TTCCTAGGGGT |
| Primer for luciferase | |
| reporter plasmid | |
| construction (5′ to 3′) | |
| (nucleic acid sequence) | |
| SEQ ID NO: 6 | CCGGAATGCCAAGCTGAGAGCAGCAGGAGG |
| HLA-C Reverse | AGGG |
| Primer for luciferase | |
| reporter plasmid | |
| construction (3′ to 5′) | |
| (nucleic acid sequence) | |
| SEQ ID NO: 7 | AGCTTGGCATTCCGGTACTG |
| pGL3-basic | |
| plasmid Forward Primer | |
| for luciferase reporter | |
| plasmid construction (5′ to 3′) | |
| (nucleic acid sequence) | |
| SEQ ID NO: 8 | CTATCGATAGAGAAATGTTCTGGCACC |
| pGL3-basic | |
| plasmid Reverse Primer for | |
| luciferase reporter plasmid | |
| construction (3′ to 5′) | |
| (nucleic acid sequence) | |
| SEQ ID NO: 9 | GTGTCACTGTTGGAGGTTATT |
| TRCN0000245004 (shGR-1) | |
| (5′ to 3′) (nucleic acid | |
| sequence) | |
| SEQ ID NO: 10 | TGGATAAGACCATGAGTATTG |
| TRCN0000245006 (shGR-2) | |
| (5′ to 3′) (nucleic acid | |
| sequence) | |
| SEQ ID NO: 11 | CCCAGAGATGTTAGCTGAAAT |
| TRCN0000026186 (5′ to 3′) | |
| (nucleic acid sequence) | |
| SEQ ID NO: 12 | TGGATAAGTCCATGAGTATTG |
| TRCN0000238464 (5′ to 3′) | |
| (nucleic acid sequence) | |
| SEQ ID NO: 13 | CCAGTTTCTAATATGCTATAC |
| TRCN0000295705 (5′ to 3′) | |
| (nucleic acid sequence) | |
| SEQ ID NO: 14 | TCAAGGAGCAGTTTCTGGACGG |
| CALR Forward 5′ to 3′ | |
| Primer | |
| SEQ ID NO: 15 | GCATCCTGGCTTGTCTGCAAAC |
| CALR Reverse 5′ to 3′ | |
| Primer | |
| SEQ ID NO: 16 | GCTGGTTAGATGATGAGCCTGAG |
| CANX Forward 5′ to 3′ | |
| Primer | |
| SEQ ID NO: 17 | ACACCACATCCAGGAGCTGACT |
| CANX Reverse 5′ to 3′ | |
| Primer | |
| SEQ ID NO: 18 | GTCTGTCAGTGTGACCCATCCT |
| ERAP1 Forward 5′ to 3′ | |
| Primer | |
| SEQ ID NO: 19 | CTGAGCAGGATTTTCCACAGGTG |
| ERAP1 Reverse 5′ to 3′ | |
| Primer | |
| SEQ ID NO: 20 | GTCAGCCACTTGAAGAAGCAGG |
| PDIA3 Forward 5′ to 3′ | |
| Primer | |
| SEQ ID NO: 21 | TAGGAACTCGGAGTGAGCCTCA |
| PDIA3 Reverse 5′ to 3′ | |
| Primer | |
| SEQ ID NO: 22 | GAGCCTGTTCTCATCACCATGG |
| TAPBP Forward 5′ to 3′ | |
| Primer | |
| SEQ ID NO: 23 | GTAGGCAAAGCTCAAGTCCAGC |
| TAPBP Reverse 5′ to 3′ | |
| Primer | |
| SEQ ID NO: 24 | GCAGTCAACTCCTGGACCACTA |
| TAP1 Forward 5′ to 3′ | |
| Primer | |
| SEQ ID NO: 25 | CAAGGTTCCCACTGCTTACAGC |
| TAP1 Reverse 5′ to 3′ | |
| Primer | |
| SEQ ID NO: 26 | ATGCCCTTCACAATAGCAGCGG |
| TAP2 Forward 5′ to 3′ | |
| Primer | |
| SEQ ID NO: 27 | CCAAAACTGCGAACGGTCTGCA |
| TAP2 Reverse 5′ to 3′ | |
| Primer | |
| SEQ ID NO: 28 | CCGCACAACCTCGGCAGGCA |
| SEC61B Forward 5′ to 3′ | |
| Primer | |
| SEQ ID NO: 29 | CAGAAGCGATGAACAGAAGACTC |
| SEC61B Reverse 5′ to 3′ | |
| Primer | |
| SEQ ID NO: 30 | GCAGTTTGTTGAGCCAAGTCG |
| SEC61G Forward 5′ to 3′ | |
| Primer | |
| SEQ ID NO: 31 | CCAGCCGAATGGAGTCCTT |
| SEC61G Reverse 5′ to 3′ | |
| Primer | |
| SEQ ID NO: 32 | AGATACACCTGCCATGTGCAGC |
| HLA-A Forward 5′ to 3′ | |
| Primer | |
| SEQ ID NO: 33 | GATCACAGCTCCAAGGAGAACC |
| HLA-A Reverse 5′ to 3′ | |
| Primer | |
| SEQ ID NO: 34 | CTGCTGTGATGTGTAGGAGGAAG |
| HLA-B Forward 5′ to 3′ | |
| Primer | |
| SEQ ID NO: 35 | GCTGTGAGAGACACATCAGAGC |
| HLA-B Reverse 5′ to 3′ | |
| Primer | |
| SEQ ID NO: 36 | GGAGACACAGAAGTACAAGCGC |
| HLA-C Forward 5′ to 3′ | |
| Primer | |
| SEQ ID NO: 37 | ACATCCTCTGGAGGGTGTGAGA |
| HLA-C Reverse 5′ to 3′ | |
| Primer | |
| SEQ ID NO: 38 | CCACTGAAAAAGATGAGTATGCCT |
| B2M Forward 5′ to 3′ | |
| Primer | |
| SEQ ID NO: 39 | CCAATCCAAATGCGGCATCTTCA |
| B2M Reverse 5′ to 3′ | |
| Primer | |
| SEQ ID NO: 40 | AAGGTGAAGGTCGGAGTCAAC |
| GAPDH Forward 5′ to 3′ | |
| Primer | |
| SEQ ID NO: 41 | GAAGGGGTCATTGATGGCAAC |
| GAPDH Reverse 5′ to 3′ | |
| Primer | |
| SEQ ID NO: 42 | CCATCAGCTTGTCTGTTTCATTCC |
| CD86 Forward 5′ to 3′ | |
| Primer | |
| SEQ ID NO: 43 | GCTGTAATCCAAGGAATGTGGTC |
| CD86 Reverse 5′ to 3′ | |
| Primer | |
| SEQ ID NO: 44 | GTTTCACTGCCTGGTGTTGAGC |
| ICOSL Forward 5′ to 3′ | |
| Primer | |
| SEQ ID NO: 45 | ACGACGGGCACGCTGAAGTTTG |
| ICOSL Reverse 5′ to 3′ | |
| Primer | |
| SEQ ID NO: 46 | CTGGCTTTCGTGTGCTGGAGAA |
| CD276 Forward 5′ to 3′ | |
| Primer | |
| SEQ ID NO: 47 | GCTGTCAGAGTGTTTCAGAGGC |
| CD276 Reverse 5′ to 3′ | |
| Primer | |
| SEQ ID NO: 48 | CTCACAGATGCTGGCACCTACA |
| B7H4 Forward 5′ to 3′ | |
| Primer | |
| SEQ ID NO: 49 | GCAAGGTCTCTGAGCTGGCATT |
| B7H4 Reverse 5′ to 3′ | |
| Primer | |
| SEQ ID NO: 50 | CCTGGAGGATGAAGGGTGTTAC |
| CD200 Forward 5′ to 3′ | |
| Primer | |
| SEQ ID NO: 51 | AGTGAAGGGATACTATGGGCTGT |
| CD200 Reverse 5′ to 3′ | |
| Primer | |
| SEQ ID NO: 52 | TTCGCACAGGCTCAGCAGCAG |
| CD70 Forward 5′ to 3′ | |
| Primer | |
| SEQ ID NO: 53 | TTGTCCAGCTCTGGTCCATGCA |
| CD70 Reverse 5′ to 3′ | |
| Primer | |
| SEQ ID NO: 54 | TGCCGACTACAAGCGAATTACTG |
| CD274 Forward 5′ to 3′ | |
| Primer | |
| SEQ ID NO: 55 | CTGCTTGTCCAGATGACTTCGG |
| CD274 Reverse 5′ to 3′ | |
| Primer | |
| SEQ ID NO: 56 | CTCGTTCCACATACCTCAAGTCC |
| PDL2 Forward 5′ to 3′ | |
| Primer | |
| SEQ ID NO: 57 | CTGGAACCTTTAGGATGTGAGTG |
| PDL2 Reverse 5′ to 3′ | |
| Primer | |
| SEQ ID NO: 58 | CACTGTCACCAGCCTCTGGATA |
| CD155 Forward 5′ to 3′ | |
| Primer | |
| SEQ ID NO: 59 | TCATAGCCAGAGATGGATACCTC |
| CD 155 Reverse 5′ to 3′ | |
| Primer | |
| SEQ ID NO: 60 | ACACCCAGATCGACAACTCCTG |
| GALECTIN9 Forward 5′ to | |
| 3′ Primer | |
| SEQ ID NO: 61 | CAAACAGGTGCTGACCATCCAC |
| GALECTIN9 Reverse 5′ to | |
| 3′ Primer | |
| SEQ ID NO: 62 | TATCCTCGCTGTGGTTGGACTG |
| CD47 Forward 5′ to 3′ | |
| Primer | |
| SEQ ID NO: 63 | TAGTCCAAGTAATTGTGCTAGAGC |
| CD48 Reverse 5′ to 3′ | |
| Primer | |
| SEQ ID NO: 64 | CACGCCAATAACTCAGTCACTGG |
| CEACAM1 Forward 5′ to 3′ | |
| Primer | |
| SEQ ID NO: 65 | TTGTGGAGCAGGTCAGGTTCAC |
| CEACAM2 Reverse 5′ to 3′ | |
| Primer | |
| SEQ ID NO: 66 | TCTTCATGGTCACCACGGCTGT |
| BTN2A1 Forward 5′ to 3′ | |
| Primer | |
| SEQ ID NO: 67 | ACAGGGAGACACACTGGGCATA |
| BTN2A1 Reverse 5′ to 3′ | |
| Primer | |
| SEQ ID NO: 68 | CACCACATACCTGCTTGTCAGC |
| TL1A Forward 5′ to 3′ | |
| Primer | |
| SEQ ID NO: 69 | TCTCCGTCTGCTCTAAGAGGTG |
| TL1A Reverse 5′ to 3′ | |
| Primer | |
| SEQ ID NO: 70 | GGCTGGAGTCTACTATGTCTTCT |
| CD137L Forward 5′ to 3′ | |
| Primer | |
| SEQ ID NO: 71 | ACCTCGGTGAAGGGAGTCC |
| CD137L Reverse 5′ to 3′ | |
| Primer | |
| SEQ ID NO: 72 | AAACTCGCATCTACTGGCAAA |
| CD80 Forward 5′ to 3′ | |
| Primer | |
| SEQ ID NO: 73 | GGTTCTTGTACTCGGGCCATA |
| CD80 Reverse 5′ to 3′ | |
| Primer | |
| SEQ ID NO: 74 | GTGCAGTCCAGGTTATCGTGT |
| HVEM Forward 5′ to 3′ | |
| Primer | |
| SEQ ID NO: 75 | CACTTGCTTAGGCCATTGAGG |
| HVEM Reverse 5′ to 3′ | |
| Primer | |
| SEQ ID NO: 76 | GGCAGGGTCAGACTTGATCC |
| CD48 Forward 5′ to 3′ | |
| Primer | |
| SEQ ID NO: 77 | GTAGGTGCTGTTGTCCTCTTTC |
| CD48 Reverse 5′ to 3′ | |
| Primer | |
| SEQ ID NO: 78 | TACAAAGGCAGTGACCATTIGG |
| B7H7 Forward 5′ to 3′ | |
| Primer | |
| SEQ ID NO: 79 | AGGTGTAAATTCCTTCGTCCAGA |
| B7H7 Reverse 5′ to 3′ | |
| Primer | |
| SEQ ID NO: 80 | AGATGCACCATCCAACTGTGTGG |
| VISTA Forward 5′ to 3′ | |
| Primer | |
| SEQ ID NO: 81 | AGGCAGAGGATTCCTACGATGC |
| VISTA Reverse 5′ to 3′ | |
| Primer | |
| SEQ ID NO: 82 | GCCTGATCTCATAGAGTCTGGC |
| IDO Forward 5′ to 3′ | |
| Primer | |
| SEQ ID NO: 83 | TGCATCCCAGAACTAGACGTGC |
| IDO Reverse 5′ to 3′ | |
| Primer | |
| SEQ ID NO: 84 | TACCCTGCCCGAGTCTTCTG |
| TDO Forward 5′ to 3′ | |
| Primer | |
| SEQ ID NO: 85 | TGGTGGTAGGTGTAGGGGTTC |
| TDO Reverse 5′ to 3′ | |
| Primer | |
| SEQ ID NO: 86 | CGCGGATCGTCAACATCTC |
| SIGLECT15 Forward 5′ to | |
| 3′ Primer | |
| SEQ ID NO: 87 | GTTCGGCGGTCACTAGGTG |
| SIGLECT15 Reverse 5′ to | |
| 3′ Primer | |
| SEQ ID NO: 88 | CCAGGCCAAGATTCGAGAGG |
| CD252 Forward 5′ to 3′ | |
| Primer | |
| SEQ ID NO: 89 | CCGATGTGATACCTGAAGAGCA |
| CD252 Reverse 5′ to 3′ | |
| Primer | |
| SEQ ID NO: 90 | AGTGGCTCCCAATGCAAACTA |
| GITRL Forward 5′ to 3′ | |
| Primer | |
| SEQ ID NO: 91 | TATACAGCCGCACCTCAAAAG |
| GITRL Reverse 5′ to 3′ | |
| Primer | |
| SEQ ID NO: 92 | TGCCGCTATCGAAAATGTCTT |
| NR3C1 Forward 5′ to 3′ | |
| Primer | |
| SEQ ID NO: 93 | GGGTAGGGGTGAGTTGTGGT |
| NR3C1 Reverse 5′ to 3′ | |
| Primer | |
| SEQ ID NO: 94 | TGCGGACTACAAGCGAATCACG |
| Cd274 Forward 5′ to 3′ | |
| Primer | |
| SEQ ID NO: 95 | CTCAGCTTCTGGATAACCCTCG |
| Cd274 Reverse 5′ to 3′ | |
| Primer | |
| SEQ ID NO: 96 | ACAGTTCCACCCGCCTCACATT |
| B2m Forward 5′ to 3′ | |
| Primer | |
| SEQ ID NO: 97 | TAGAAAGACCAGTCCTTGCTGAAG |
| B2m Reverse 5′ to 3′ | |
| Primer | |
| SEQ ID NO: 98 H- | GGCAATGAGCAGAGTTTCCGAG |
| 2k Forward 5′ to 3′ Primer | |
| SEQ ID NO: 99 H- | CCACTTCACAGCCAGAGATCAC |
| 2k Reverse 5′ to 3′ Primer | |
| SEQ ID NO: 100 | TGAGGAACCTGCTCGGCTACTA |
| H-2d Forward 5′ to 3′ | |
| Primer | |
| SEQ ID NO: 101 | GGTCTTCGTTCAGGGCGATGTA |
| H-2d Reverse 5′ to 3′ | |
| Primer | |
| SEQ ID NO: 102 | CCCTATCTCTGATACCGTTGTCC |
| Fn1 Forward 5′ to 3′ | |
| Primer | |
| SEQ ID NO: 103 | TGCCGCAACTACTGTGATTCGG |
| Fn1 Reverse 5′ to 3′ Primer | |
| SEQ ID NO: 104 | CTACAGTGGCTGTGGGAAAGTC |
| Klf9 Forward 5′ to 3′ | |
| Primer | |
| SEQ ID NO: 105 | CTCATCCGAGCGCGAGAACTTT |
| Klf9 Reverse 5′ to 3′ Primer | |
| SEQ ID NO: 106 | TCTGTGGCAAGGCTTTCTCCAG |
| Snai2 Forward 5′ to 3′ | |
| Primer | |
| SEQ ID NO: 107 | TGCAGATGTGCCCTCAGGTTTG |
| Snai2 Reverse 5′ to 3′ | |
| Primer | |
| SEQ ID NO: 108 | CCGGGTCCCCAGGTAAAGA |
| NR3cl Forward 5′ to 3′ | |
| Primer | |
| SEQ ID NO: 109 | TGTCCGGTAAAATAAGAGGCTTG |
| NR3cl Reverse 5′ to 3′ | |
| Primer | |
| SEQ ID NO: 110 | AGGTCGGTGTGAACGGATTTG |
| Gapdh Forward 5′ to 3′ | |
| Primer | |
| SEQ ID NO: 111 | TGTAGACCATGTAGTTGAGGTCA |
| Gapdh Reverse 5′ to 3′ | |
| Primer | |
| SEQ ID NO: 112 | CCAATGCAAGGGCTATCTCAA |
| PD-L1 Forward Primer | |
| pair #1 | |
| SEQ ID NO: 113 | CACCGTGCCTGTGTGC |
| PD-L1 Reverse Primer | |
| pair #1 | |
| SEQ ID NO: 114 | AAAAGGGAGCACACAGGCAC |
| PD-L1 Forward Primer | |
| pair #2 | |
| SEQ ID NO: 115 | AGTATTATCCCGCGCTGAAC |
| PD-L1 Reverse Primer | |
| pair #2 | |
| SEQ ID NO: 116 | AGAAGTTCAGCGCGGGATAA |
| PD-L1 Forward Primer | |
| pair #3 | |
| SEQ ID NO: 117 | GGCTGCGGAAGCCTATTCT |
| PD-L1 Reverse Primer | |
| pair #3 | |
| SEQ ID NO: 118 | AAGATGTAGCTCGGGATGGGA |
| PD-L1 Forward Primer | |
| pair #4 | |
| SEQ ID NO: 119 | ATCGTGGATTCTGTGACTTCC |
| PD-L1 Reverse Primer | |
| pair #4 | |
| SEQ ID NO: 120 | GGAAGTCACAGAATCCACGA |
| PD-L1 Forward Primer | |
| pair #5 | |
| SEQ ID NO: 121 | AAAGTCAGCAGCAGACCCAT |
| PD-L1 Reverse Primer | |
| pair #5 | |
| SEQ ID NO: 122 | GTGCGTTCAGATGTTGGCTT |
| PD-L1 Forward Primer | |
| pair #6 | |
| SEQ ID NO: 123 | ATTTTCACCGGGAAGAGTTTCG |
| PD-L1 Reverse Primer | |
| pair #6 | |
| SEQ ID NO: 124 | CTAAACTGAAAGCTTCCGCCG |
| PD-L1 Forward Primer | |
| pair #7 | |
| SEQ ID NO: 125 | CTCTGCCCAAGGCAGCAAATC |
| PD-L1 Reverse Primer | |
| pair #7 | |
| SEQ ID NO: 126 | TGAGGGGTTTCCTCCACCAT |
| HLA-A Forward Primer #1 | |
| SEQ ID NO: 127 | CTCGGCCTCCCAAAGTGT |
| HLA-A Reverse Primer #1 | |
| SEQ ID NO: 128 | GCCACTGTACCCCAGCCT |
| HLA-A Forward Primer #2 | |
| SEQ ID NO: 129 | GCTCACCAGCGTTCTTTCCT |
| HLA-A Reverse Primer #2 | |
| SEQ ID NO: 130 | CAAAATGAAAACACAGCTTTTGG |
| HLA-A Forward Primer #3 | |
| SEQ ID NO: 131 | GGCAAAACAACACACATTTATCT |
| HLA-A Reverse Primer #3 | |
| SEQ ID NO: 132 | TGTGATGCAATTTAATACACTC |
| HLA-A Forward Primer #4 | |
| SEQ ID NO: 133 | ACTTGAGCATATGAGGGGAT |
| HLA-A Reverse Primer #4 | |
| SEQ ID NO: 134 | ATTGGGACTGCATGGAGCACT |
| HLA-B Forward Primer #1 | |
| SEQ ID NO: 135 | TTTCCCTGTGTGAGTCCAGAAC |
| HLA-B Reverse Primer #1 | |
| SEQ ID NO: 136 | TACAGATCCACAGTCTGGGAAA |
| HLA-B Forward Primer #2 | |
| SEQ ID NO: 137 | AAAAGACCTGTGTCAAAACAGCAT |
| HLA-B Reverse Primer #2 | |
| SEQ ID NO: 138 | GCTGTTTTGACACAGGTCTTTTAC |
| HLA-B Forward Primer #3 | |
| SEQ ID NO: 139 | AGGGATTCTCATAGAGACCAGTT |
| HLA-B Reverse Primer #3 | |
| SEQ ID NO: 140 | CTGGTTTCCACAGACAGATCCT |
| HLA-B Forward Primer #4 | |
| SEQ ID NO: 141 | TGATCCCTCTTCTCCTACACCA |
| HLA-B Reverse Primer #4 | |
| SEQ ID NO: 142 | AAGAGCATTGGGACTGCATGG |
| HLA-C Forward Primer #1 | |
| SEQ ID NO: 143 | GTGTGAGTCCAGAACATCTCC |
| HLA-C Reverse Primer #1 | |
| SEQ ID NO: 144 | ACCCAGAGTCACAGAACCAT |
| HLA-C Forward Primer #2 | |
| SEQ ID NO: 145 | ATTCCAATCTGTGAAAGACCTGTG |
| HLA-C Reverse Primer #2 | |
| SEQ ID NO: 146 | ACCAATATTGTGCTACCTACTGT |
| HLA-C Forward Primer #3 | |
| SEQ ID NO: 147 | AGGAGTTTTCTCTTTAAACCTGGTG |
| HLA-C Reverse Primer #3 | |
| SEQ ID NO: 148 | AGTCCAAGGGGAGAGGTAAGTTT |
| HLA-C Forward Primer #4 | |
| SEQ ID NO: 149 | GTCCCTGAACTGGACTCCCTG |
| HLA-C Reverse Primer #4 | |
| SEQ ID NO: 150 | TTGGGGTCTCTCCCTGGTTTC |
| HLA-C Forward Primer #5 | |
| SEQ ID NO: 151 | TCCTGATCCCTCTTCTCCTACA |
| HLA-C Reverse Primer #5 | |
| SEQ ID NO: 152 | ACTAAAATTGCCGAGCCCTTTG |
| B2M Forward Primer #1 | |
| SEQ ID NO: 153 | CCATTCATTTTATGGACAGGGGA |
| B2M Reverse Primer #1 | |
| SEQ ID NO: 154 | ACAGCAAGGACATAGGGAGG |
| B2M Forward Primer #2 | |
| SEQ ID NO: 155 | TGCTTCTTGTATCCCTTCAGGA |
| B2M Reverse Primer #2 | |
| SEQ ID NO: 156 | AGGACCTTCTCTGAGCTGTC |
| B2M Forward Primer #3 | |
| SEQ ID NO: 157 | GGGTTTCTCCATTCTCTGGGT |
| B2M Reverse Primer #3 | |
| SEQ ID NO: 158 | ACAGAAGTTCTCCTTCTGCTAGG |
| B2M Forward Primer #4 | |
| SEQ ID NO: 159 | AGTTAACTGCTCGGACCTGC |
| B2M Reverse Primer #4 | |
| SEQ ID NO: 160 | AATGCAGGTCCGAGCAGTTA |
| B2M Forward Primer #5 | |
| SEQ ID NO: 161 | TCAGGCTGGAGGCACATTAAG |
| B2M Reverse Primer #5 | |

What is claimed is:

1. A method of treating a subject having a pancreatic cancer that does not respond to an immune checkpoint inhibitor (ICI) therapy comprising administering to the subject in need thereof, a combination therapy comprising one or more glucocorticoid receptor antagonists, or pharmaceutically acceptable salts thereof, and one or more immune checkpoint inhibitor selected from the group consisting of a PD-1 antagonist, a PD-L1 antagonist, a CTLA-4 antagonist, or any combination thereof, thereby treating the subject in need thereof.

2. The method of claim 1, wherein the one or more glucocorticoid receptor antagonists, or pharmaceutically acceptable salt thereof, sensitizes the pancreatic cancer to the one or more immune checkpoint inhibitors.

3. The method of claim 1 or 2, wherein the one or more glucocorticoid receptor antagonists, or pharmaceutically acceptable salts thereof, are administered before or concomitantly with the one or more immune checkpoint inhibitors.

4. The method of any one of claims 1-3, wherein the one or more glucocorticoid receptor antagonist, or pharmaceutically acceptable salt thereof, is administered orally, intranasally, subcutaneously, intramuscularly, intradermally, intravenously, intra-arterially, parenterally, or by catherization.

5. The method of claim 4, wherein the one or more glucocorticoid receptor antagonist, or pharmaceutically acceptable salt thereof, is administered orally.

6. The method of any one of claims 1-5, wherein the one or more immune checkpoint inhibitors are administered subcutaneously, intramuscularly, intravenously, intra-arterially, parenterally, or by catherization.

7. The method of claim 6, wherein the one or more immune checkpoint inhibitors are administered intravenously.

8. The method of any one of claims 1-7, wherein the subject is administered the combination therapy as the first line of therapy.

9. The method of any one of claims 1-7, wherein the subject is administered the combination therapy after the subject has been previously treated for pancreatic cancer.

10. The method of claim 9, wherein the subject has been previously treated for pancreatic cancer by administration of an immune checkpoint inhibitor not in combination with a glucocorticoid receptor antagonist, or pharmaceutically acceptable salt thereof.

11. A method for sensitizing a pancreatic tumor to an immune checkpoint inhibitor comprising administering to a subject having the pancreatic tumor a combination therapy comprising a combination of one or more glucocorticoid receptor antagonists, or pharmaceutically acceptable salt thereof, and one or more immune checkpoint inhibitors selected from the group consisting of a PD-1 antagonist, PD-L1 antagonist, CTLA-4 antagonist, or any combination thereof, wherein the pancreatic tumor is pancreatic ductal adenocarcinoma.

12. The method of claim 11, wherein the one or more glucocorticoid receptor antagonists, or pharmaceutically acceptable salt thereof, are administered before or concomitantly with the one or more immune checkpoint inhibitors.

13. The method of claim 11 or 12, wherein the one or more glucocorticoid receptor antagonist, or pharmaceutically acceptable salt thereof, is administered orally, intranasally, subcutaneously, intramuscularly, intradermally, intravenously, intra-arterially, parenterally, or by catherization.

14. The method of claim 13, wherein the one or more glucocorticoid receptor antagonist, or pharmaceutically acceptable salt thereof, is administered orally.

15. The method of any one of claims 11-14, wherein the one or more immune checkpoint inhibitors are administered subcutaneously, intramuscularly, intravenously, intra-arterially, parenterally, or by catherization.

16. The method of claim 15, wherein the one or more immune checkpoint inhibitors are administered intravenously.

17. The method of any one of claims 11-16, wherein the patient is administered the combination therapy as the first line of therapy.

18. The method of any one of claims 11-16, wherein the subject is administered the combination therapy after the subject has been previously treated for pancreatic cancer.

19. The method of claim 18, wherein the subject has been previously treated for pancreatic cancer by administration of an immune checkpoint inhibitor not in combination with a glucocorticoid receptor antagonist, or pharmaceutically acceptable salt thereof.

20. The method of any one of claims 1-19, wherein the pancreatic cancer or pancreatic tumor is pancreatic ductal adenocarcinoma.

21. The method of any one of claims 1-20, wherein the one or more glucocorticoid receptor antagonist, or pharmaceutically acceptable salt thereof, is administered in a total daily amount of about 100 mg to about 2500 mg.

22. The method of any one of claims 1-21, wherein the one or more glucocorticoid receptor antagonist, or pharmaceutically acceptable salt thereof, is administered as a solid oral dosage form.

23. The method of claim 22, wherein the solid oral dosage form is a capsule or tablet.

24. The method of any one of claims 1-23, wherein the glucocorticoid receptor antagonist is in a pharmaceutically acceptable form.

25. The method of claim 24, wherein the glucocorticoid receptor antagonist is a pharmaceutically acceptable salt form of mifepristone.

26. The method of any one of claims 1-23, wherein the glucocorticoid receptor antagonist is in free base form.

27. The method of claim 26, wherein the glucocorticoid receptor antagonist is mifepristone.

28. The method of any one of claims 1-27, wherein the one or more immune checkpoint inhibitors comprises or is the PD-1 antagonist.

29. The method of any one of claims 1-27, wherein the one or more immune checkpoint inhibitors comprises or is the PD-L1 antagonist.

30. The method of any one of claims 1-27, wherein the one or more immune checkpoint inhibitors comprises or is the CTLA-4 antagonist.

31. The method of any one of claims 1-27, wherein the one or more immune checkpoint inhibitors are the PD-1 antagonist and the CTLA-4 antagonist.

32. The method of any one of claims 1-27, wherein the one or more immune checkpoint inhibitors are the PD-L1 antagonist and the CTLA-4 antagonist.

33. The method of any one of claims 1-27, wherein the one or more checkpoint inhibitors are the PD-1 antagonist, the PD-L1 antagonist, and the CTLA-4 antagonist.

34. The method of any one of claims 1-33, wherein the one or more immune checkpoint inhibitors comprise an antibody.

35. The method of any one of claims 1-28, 31, 33, and 34, wherein the one or more PD-1 antagonist is selected from the group consisting of pembrolizumab, nivolumab, cemiplimab, sintilimab, dostarlimab, tisleizumab, torpipalimab, spartalizumab, camrelizumab, lambrolizumab, AMP-224, pidilizumab, or any combinations thereof.

36. The method of claim 35, wherein the PD-1 antagonist is pembrolizumab.

37. The method of claim 36, wherein the pembrolizumab is administered at a single dose of about 100 mg to about 600 mg.

38. The method of claim 37, wherein the single dose is administered once weekly, once every two weeks, once every three weeks, once every four weeks, once every five weeks, or once every six weeks.

39. The method of claim 38, wherein the single dose is about 200 mg administered once every three weeks.

40. The method of claim 38, wherein the single dose is about 400 mg administered once every six weeks.

41. The method of claim 35, wherein the PD-1 antagonist is nivolumab.

42. The method of claim 41, wherein the nivolumab is administered at a single dose of about 0.5 mg/kg to about 7.5 mg/kg.

43. The method of claim 41, wherein the nivolumab is administered at a single dose of about 100 mg to about 750 mg.

44. The method of claim 42 or 43, wherein the single dose is administered once weekly, once every two weeks, once every three weeks, once every four weeks, once every five weeks, or once every six weeks.

45. The method of claim 44, wherein the single dose is about 1 mg/kg administered once every three weeks.

46. The method of claim 44, wherein the single dose is about 3 mg/kg administered once every two weeks.

47. The method of claim 44, wherein the single dose is about 3 mg/kg administered once every three weeks.

48. The method of claim 44, wherein the single dose is about 240 mg administered once every two weeks.

49. The method of claim 44, wherein the single dose is about 360 mg administered once every three weeks.

50. The method of claim 35, wherein the PD-1 antagonist is cemiplimab.

51. The method of claim 50, wherein the cemiplimab is administered at a single dose of about 100 mg to about 500 mg.

52. The method of claim 51, wherein the single dose is administered once weekly, once every two weeks, once every three weeks, once every four weeks, once every five weeks, or once every six weeks.

53. The method of claim 52, wherein the single dose is about 350 mg administered once every three weeks.

54. The method of claim 35, wherein the PD-1 antagonist is sintilimab.

55. The method of claim 54, wherein the sintilimab is administered at a single dose of about 25 mg to about 500 mg.

56. The method of claim 55, wherein the single dose is administered once weekly, once every two weeks, once every three weeks, once every four weeks, once every five weeks, or once every six weeks.

57. The method of claim 56, wherein the single dose is about 100 mg administered once every three weeks.

58. The method of claim 56, wherein the single dose is about 200 mg administered once every three weeks.

59. The method of claim 35, wherein the PD-1 antagonist is dostarlimab.

60. The method of claim 59, wherein the dostarlimab is administered at a single dose of about 100 mg to about 1500 mg.

61. The method of claim 60, wherein the single dose is administered once weekly, once every two weeks, once every three weeks, once every four weeks, once every five weeks, or once every six weeks.

62. The method of claim 61, wherein the single dose is about 500 mg administered once every three weeks.

63. The method of claim 61, wherein the single dose is about 1000 mg administered once every six weeks.

64. The method of claim 61, wherein the single dose is about 500 mg administered once every three weeks for the first four doses and then about 1000 mg every six weeks.

65. The method of claim 35, wherein the PD-1 antagonist is tislelizumab.

66. The method of claim 65, wherein the tislelizumab is administered at a single dose of about 50 mg to about 500 mg.

67. The method of claim 66, wherein the single dose is administered once weekly, once every two weeks, once every three weeks, once every four weeks, once every five weeks, or once every six weeks.

68. The method of claim 67, wherein the single dose is about 200 mg administered once every three weeks.

69. The method of claim 35, wherein the PD-1 antagonist is toripalimab.

70. The method of claim 69, wherein the toripalimab is administered at a single dose of about 1 mg/kg to about 7 mg/kg.

71. The method of claim 70, wherein the single dose is administered once weekly, once every two weeks, once every three weeks, once every four weeks, once every five weeks, or once every six weeks.

72. The method of claim 70, wherein the single dose is about 3 mg/kg administered once every two weeks.

73. The method of claim 35, wherein the PD-1 antagonist is spartalizumab.

74. The method of claim 73, wherein the spartalizumab is administered at a single dose of about 100 mg to about 700 mg.

75. The method of claim 73, wherein the single dose is administered once weekly, once every two weeks, once every three weeks, once every four weeks, once every five weeks, or once every six weeks.

76. The method of claim 75, wherein the spartalizumab is administered at single dose of about 300 mg every three weeks.

77. The method of claim 75, wherein the spartalizumab is administered at a single dose of about 400 mg every four weeks.

78. The method of claim 35, wherein the PD-1 antagonist is camrelizumab.

79. The method of claim 78, wherein the camrelizumab is administered at a single dose of about 50 mg to about 500 mg.

80. The method of claim 79, wherein the single dose is administered once weekly, once every two weeks, once every three weeks, once every four weeks, once every five weeks, or once every six weeks.

81. The method of claim 80, wherein the single dose is about 200 mg administered once every three weeks.

82. The method of claim 35, wherein the PD-1 antagonist is lambrolizumab.

83. The method of claim 82, wherein the lambrolizumab is administered at a single dose of about 0.5 mg/kg to about 15 mg.

84. The method of claim 83, wherein the single dose is administered once weekly, once every two weeks, once every three weeks, once every four weeks, once every five weeks, or once every six weeks.

85. The method of claim 35, wherein the PD-1 antagonist is AMP-224.

86. The method of claim 85, wherein the AMP-224 is administered at a single dose of about 1 mg/kg to about 15 mg/kg.

87. The method of claim 86, wherein the single dose is administered once weekly, once every two weeks, once every three weeks, once every four weeks, once every five weeks, or once every six weeks.

88. The method of claim 35, wherein the PD-1 antagonist is pidilizumab.

89. The method of claim 88, wherein the pidilizumab is administered at a single dose of about 0.5 mg/kg to about 5 mg/kg.

90. The method of claim 89, wherein the single dose is administered once weekly, once every two weeks, once every three weeks, once every four weeks, once every five weeks, or once every six weeks.

91. The method of any one of claims 1-27, 29, and 32-34, wherein the one or more PD-L1 antagonist is selected from the group consisting of atezolizumab, durvalumab, avelumab, KN035, MDX-1105, MEDI4736, MPDL3280A, BMS-936559, and combinations thereof.

92. The method of claim 91, wherein the PD-L1 antagonist is atezolizumab.

93. The method of claim 92, wherein the atezolizumab is administered at single dose of about 500 mg to about 2500 mg.

94. The method of claim 93, wherein the single dose is administered once weekly, once every two weeks, once every three weeks, once every four weeks, once every five weeks, or once every six weeks.

95. The method of claim 94, wherein the single dose is about 840 mg administered once every two weeks.

96. The method of claim 94, wherein the single dose is about 1200 mg administered once every three weeks.

97. The method of claim 94, wherein the single dose is about 1680 mg administered once every four weeks.

98. The method of claim 91, wherein the PD-L1 antagonist is durvalumab.

99. The method of claim 98, wherein the durvalumab is administered at a single dose of about 750 mg to about 2500 mg.

100. The method of claim 99, wherein the single dose is administered once weekly, once every two weeks, once every three weeks, once every four weeks, once every five weeks, or once every six weeks.

101. The method of claim 100, wherein the single dose is about 1500 mg administered once every three weeks.

102. The method of claim 100, wherein the single dose is about 1500 mg administered once every four weeks.

103. The method of claim 91, wherein the PD-L1 antagonist is avelumab.

104. The method of claim 103, wherein the avelumab is administered at a single dose of about 100 mg to about 1500 mg.

105. The method of claim 104, wherein the single dose is administered once weekly, once every two weeks, once every three weeks, once every four weeks, once every five weeks, or once every six weeks.

106. The method of claim 105, wherein the single dose is about 800 mg administered once every two weeks.

107. The method of claim 91, wherein the PD-L1 antagonist is KN035.

108. The method of claim 107, wherein the KN035 is administered at a single dose of about 50 mg to about 750 mg.

109. The method of claim 108, wherein the single dose is administered once weekly, once every two weeks, once every three weeks, once every four weeks, once every five weeks, or once every six weeks.

110. The method of claim 109, wherein the single dose is about 300 mg administered once every week.

111. The method of claim 91, wherein the PD-L1 antagonist is MEDI4736.

112. The method of claim 111, wherein the MEDI4736 is administered at a single dose of about 0.1 mg/kg to about 20 mg/kg.

113. The method of claim 112, wherein the single dose is administered once weekly, once every two weeks, once every three weeks, once every four weeks, once every five weeks, or once every six weeks.

114. The method of claim 91, wherein the PD-L1 antagonist is MPDL3280A.

115. The method of claim 114, wherein the MPDL3280A is administered at a single dose of about 750 mg to about 1500 mg.

116. The method of claim 115, wherein the single dose is administered once weekly, once every two weeks, once every three weeks, once every four weeks, once every five weeks, or once every six weeks.

117. The method of claim 91, wherein the PD-L1 antagonist is BMS-936559.

118. The method of claim 117, wherein the BMS-936559 is administered at a single dose of about 0.01 mg/kg to about 10 mg/kg.

119. The method of claim 118, wherein the single dose is administered once weekly, once every two weeks, once every three weeks, once every four weeks, once every five weeks, or once every six weeks.

120. The method of any one of claims 1-27 and 30-34, wherein the one or more CTLA-4 antagonist is selected from the group consisting of ipilimumab, BMS-986218, AGEN1181, tremelimumab, and combinations thereof.

121. The method of claim 120, wherein the CTLA-4 antagonist is ipilimumab.

122. The method of claim 121, wherein the ipilimumab is administered at a single dose of about 0.25 mg/kg to about 15 mg/kg.

123. The method of claim 122, wherein the single dose is administered once weekly, once every two weeks, once every three weeks, once every four weeks, once every five weeks, once every six weeks, once every seven weeks, once every eight weeks, once every nine weeks once every ten weeks, once every 11 weeks, or once every 12 weeks.

124. The method of claim 123, wherein the single dose is about 1 mg/kg administered once every three weeks.

125. The method of claim 123, wherein the single dose is about 3 mg/kg administered once every three weeks.

126. The method of claim 123, wherein the single dose is about 10 mg/kg administered once every three weeks.

127. The method of claim 123, wherein the single dose is about 10 mg/kg administered once every 12 weeks.

128. The method of claim 120, wherein the CTLA-4 antagonist is BMS-986218.

129. The method of claim 128, wherein the BMS-986218 is administered in a single dose of about 0.5 mg to about 100 mg.

130. The method of claim 129, wherein the single dose is administered once weekly, once every two weeks, once every three weeks, once every four weeks, once every five weeks, once every six weeks, once every seven weeks, once every eight weeks, once every nine weeks once every ten weeks, once every 11 weeks, or once every 12 weeks.

131. The method of claim 130, wherein the single dose is administered once every four weeks.

132. The method of claim 120, wherein the CTLA-4 antagonist is AGEN1181.

133. The method of claim 132, wherein the AGEN1181 is administered at a single dose of about 0.05 mg/kg to about 10 mg/kg.

134. The method of claim 133, wherein the single dose is administered once weekly, once every two weeks, once every three weeks, once every four weeks, once every five weeks, once every six weeks, once every seven weeks, once every eight weeks, once every nine weeks once every ten weeks, once every 11 weeks, or once every 12 weeks.

135. The method of claim 134, wherein the single dose is about 0.1 mg/kg to about 4 mg/kg administered once every three weeks.

136. The method of claim 134, wherein the single dose is about 1 mg/kg to about 4 mg/kg administered once every six weeks.

137. The method of claim 120, wherein the CTLA-4 antagonist is tremelimumab.

138. The method of claim 137, wherein the tremelimumab is administered as a single dose of about 1 mg/kg to about 15 mg/kg.

139. The method of claim 138, wherein the single dose is administered once weekly, once every two weeks, once every three weeks, once every four weeks, once every five weeks, once every six weeks, once every seven weeks, once every eight weeks, once every nine weeks once every ten weeks, once every 11 weeks, or once every 12 weeks.

140. The method of any one of claims 1-139, wherein the tumor weight is reduced by at least 60%.

141. The method of claim 140, wherein the tumor weight is reduced by at least 75%.

142. The method of claim 141, wherein the tumor weight is reduced by at least 80%.

143. The method of any of the previous claims, wherein the pancreatic cancer or pancreatic tumor does not comprise a microsatellite instability-high tumor.
